(19) United States
(12) Patent Application Publication
(10) Pub. No.: US 2023/0364254 A1
(43) Pub. Date: Nov. 16, 2023

(54) METHODS FOR TREATING CANCERS WITH ANTIBODY DRUG CONJUGATES (ADC) THAT BIND TO 191P4D12 PROTEINS

(71) Applicants: AGENSYS, INC., Northbrook, IL (US); SEAGEN INC., Bothell, WA (US)

(72) Inventors: Elaina Marie GARTNER, Lake Forest Park, WA (US); Eric John CHOWN, Lake Forest Park, WA (US); Tina KIM-HAFKEN, Bellevue, WA (US); Dana Ann KENNEDY, Kirkland, WA (US)

(73) Assignees: AGENSYS, INC., Northbrook, IL (US); SEAGEN INC., Bothell, WA (US)

(21) Appl. No.: 18/030,225

(22) PCT Filed: Oct. 8, 2021
(86) PCT No.: PCT/US2021/054084

§ 371 Date: Apr. 4, 2023

Related U.S. Application Data
(60) Provisional application No. 63/193,493, filed on May 26, 2021.
(60) Provisional application No. 63/148,038, filed on Feb. 10, 2021.
(60) Provisional application No. 63/090,272, filed on Oct. 11, 2020.

(51) Int. Cl. A61K 47/68; A61P 35/00; G01N 33/50

(52) U.S. Cl. CPC A61K 47/68031; A61K 47/6843; A61P 35/00; G01N 33/5091; G01N 2800/7028; G01N 2800/52

(57) ABSTRACT

Provided herein are methods for treating cancers with antibody drug conjugates (ADC) that bind to 191P4DI2 protein (Nectin-4).

### CROSS-REFERENCE TO RELATED APPLICATIONS

This application claims the benefit of U.S. Application No. 63/090,272, filed Oct. 11, 2020, U.S. Application No. 63/148,038, filed Feb. 10, 2021, and U.S. Application No. 63/193,493, filed May 26, 2021, the disclosure of each of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name of “14369-270-228_SEQ_LISTING.txt” and a creation date of Sep. 23, 2021 and having a size of 39,755 bytes. The sequence listing submitted via EFS-Web is part of the specification and is herein incorporated by reference in its entirety.

### 1. FIELD

Provided herein are methods for treating cancers with antibody drug conjugates (ADC) that bind to 191P4D12 protein (Nectin-4).

### 2. BACKGROUND

Cancer is the leading cause of death in the US for people 35 to 65 years of age and it is the second leading cause of death worldwide. It was estimated in 2019 that there would be approximately 1.7 million new cancer cases and approximately 610000 deaths from cancer in the US (National Cancer Institute. 2019. Cancer Stat Facts: Cancer of Any Site. seer.cancer.gov/statfacts/html/all.html. Accessed 5 Jun. 2019). Globally there were an estimated 18.1 million new cancer cases in 2018 and approximately 9.6 million deaths attributed to cancer in 2018 (World Health Organization. Press Release. September 2018. wio.int/cancer/PRGlobocanFinal.pdf. Accessed 5 Jun. 2019). Most deaths now occur in patients with metastatic cancers. In fact, in the last 20 years, advances in treatment, including surgery, radiotherapy and adjuvant chemotherapy cured most patients with localized cancer. Patients whose cancer presented or recurred as metastatic disease obtained only modest benefit from conventional therapies in terms of overall survival (OS) and were rarely cured.

New therapeutic strategies for advanced and/or metastatic cancers include targeting molecular pathways important for cancer cell survival and novel cytotoxic compounds. The benefit of these novel drugs is reflected in prolonged survival; however, the outcome for most patients with distant metastases is still poor and novel therapies are needed.

191P4D12 (which is also known as Nectin-4) is a 66 kDa type I transmembrane protein that belongs to the nectin family of adhesion molecules. It is composed of an extracellular domain (ECD) containing 3 immunoglobulin (Ig)-like subdomains, a transmembrane helix, and an intracellular region (Takai et al., Annu Rev Cell Dev Biol (2008); 24: 309-42). Nectins are thought to mediate Ca²⁺-independent cell-cell adhesion via both homophilic and heterophilic trans-interactions at adherens junctions where they can recruit cadherins and modulate cytoskeletal rearrangements (Rikitake et al., Cell Mol Life Sci (2008); 65(2): 253-63.). Sequence identity of Nectin-4 to other Nectin family members is low and ranges between 25%-30% in the ECD (Reymond et al., Biol Chem (2001): 276(46): 43205-15).

The 3 Ig-like subdomains in the ECD of Nectin-4 are designated V, C1 and C2. The C1 domain is responsible for cis-interaction (homodimerization), while V domains of most Nectin molecules contribute to trans-interaction and cell-cell adhesion (Mandai et al., Curr Top Dev Biol (2015); 112: 197-231; Takai et al., Nat Rev Mol Cell Biol (2008); 9(8): 603-15.).

Nectin-4 was originally identified by bioinformatics and cloned from human trachea (Reymond et al., J Biol Chem (2001) 276(46): 43205-15.). Nectin-4 was identified as markedly upregulated in urothelial cancer using suppression subtractive hybridization on a pool of urothelial cancer specimens. Characterization of expression in multiple tumor specimens, both at the ribonucleic acid (RNA) level and by immunohistochemistry (IHC), also demonstrated high levels of Nectin-4 in breast, pancreatic, lung, and other cancers (Challita-Eid et al., Cancer Res (2016); 76(10): 3003-13.).

Nectin-4 has been found to be expressed in multiple cancers, particularly urothelial, breast, lung, pancreatic, and ovarian cancers. Higher levels of expression are associated with disease progression and/or poor prognosis (Fabre-Lafay et al., BMC Cancer (2007); 7: 73).

Urothelial Cancer

According to the International Agency for Research on Cancer (IARC), urothelial cancer kills more than 165000 patients annually and is the ninth most common cancer overall worldwide. Approximately 151000 new cases of urothelial cancer are diagnosed annually in Europe, with 52000 deaths per year. Over 22000 new cases are diagnosed annually in Japan, with 7600 deaths per year (Cancer Fact Sheets: All cancers excluding Non-Melanoma Skin. International Agency for Research on Cancer 2017. Retrieved from gco.iarc.fr/today/fact-sheets-cancers?cancer=29&type=0&sex=0. Accessed 19 Dec. 2017). According to National Cancer Institute estimates, approximately 77,000 new cases of urothelial cancer were diagnosed in 2016, and more than 16,000 people died from the disease in the United States (US) alone (National Cancer Institute (2016). SEER Cancer Statistics Factsheets: Bladder cancer. https://seer.cancer.gov/statfacts/html/urinb.html Accessed: Nov. 30, 2016). Metastatic urothelial cancer has a 5-year mortality rate exceeding 85%.

Urothelial cancer is the most common type of bladder cancer (90 percent of cases), and can also be found in the urothelial cells that line the renal pelvis (where urine collects inside the kidney), ureter (tube that connects the kidneys to the bladder) and urethra.

First-line therapy for metastatic urothelial cancer in patients with sufficient renal function consists of cisplatin-based combinations, like methotrexate, vinblastine, doxorubicin, and cisplatin (MVAC) or gemcitabine plus cisplatin, which demonstrate overall response rates up to 50%, including approximately 10-15% complete response (CRs) (Bellmunt J, et al., Ann Oncol (2011)22(Suppl. 6): vi45-9.). Despite initial chemosensitivity, patients are not cured and the outcome of metastatic urothelial cancer after these regimens is poor: median time to progression is only 7 months and median overall survival (OS) is 14 months. Approximately 15% of patients survive at least 5 years and the prognosis is particularly poor among patients with visceral metastases for whom the 5-year OS rate is 7% (von der Maase H. et al., J Clin Oncol. 2005; 23:4602-8).

Almost half of urothelial cancer patients are unfit for cisplatin-containing chemotherapy due to impaired renal function, poor performance status or comorbidity (Dash et at. Cancer (2006); 107(3): 506-13). In this setting, long term survival is even lower (De Santis et al. J Clin Oncol (2009); 27(33): 5634-9). In April 2017, the Food and Drug Administration (FDA) approved the anti-programmed death-ligand 1 (PD-L1) immune checkpoint inhibitor (CPI) atezolizumab (TECENTRIQ®) as first line treatment for patients ineligible for cisplatin. The accelerated approval was based on an open-label single arm study that showed long durations of response, indicating activity in this difficult-to-treat population, with an objective response rate (ORR) of 23% that was similar across varying levels of target expression. The median OS for these patients was 15.9 months, although this is a single arm study and any OS benefit will need to be confirmed in a randomized experience (Balar et al., Lancet (2017); 389(10064): 67-76).

Pembrolizumab (Keytruda®) received accelerated approval from the FDA in May 2017 as first line treatment for patients ineligible for cisplatin. The approval was based on an open label single arm study in 370 patients showing an ORR of 29% (Keytruda Prescribing Information, Merck, May 2017).

Other options for first line cisplatin-ineligible patients typically include carboplatin-based regimens or single-agent taxane or gemcitabine (Cathomas et al., Hematol Oncol Clin North Am (2015); 29(2): 329-40.).

Few options are available for second-line treatment of metastatic disease. In the European Union, the small-molecule tubulin inhibitor vinflunine (Javlor®) was authorized in 2009 based on modest activity (overall response rate 9%), moderate survival benefit of 2 months (6.9 months for vinflunine+best supportive care (BSC) vs 4.6 months for BSC alone, hazard ratio 0.88), and a favorable safety profile (Bellmunt et al. Clin Oncol (2009); 27(27): 4454-61). In May 2016, the FDA provided accelerated approval of atezolizumab as the first salvage therapy following platinum agents for locally advanced or metastatic urothelial carcinoma in the US, followed by EU approval in September 2017. In February 2017, nivolumab (Opdivo®) became the second immunotherapy granted accelerated approval by the FDA, which was followed by EU approval in June 2017. In March and May 2017, the FDA granted accelerated approval for avelumab (Bavencio®) and durvalumab (Imfinzi™). respectively, both PD-L1 blocking antibodies indicated for the treatment of patients with locally advanced or metastatic urothelial carcinoma who have disease progression during or following platinum-containing chemotherapy or have disease progression within 12 months of neoadjuvant or adjuvant treatment with platinum-containing chemotherapy. Pembrolizumab received regular approval from the FDA in May 2017 as second-line treatment (Keytruda Prescribing Information, Merck, May 2017). The approval was based on the first randomized experience reported for a CPI in the locally advanced or metastatic post-platinum urothelial cancer setting, a phase 3 study in 542 patients showing an OS of 10.3 months as compared to 7.4 months with taxane chemotherapy or vinflunine. Additionally, ORR was 21% for pembrolizumab and 11% for chemotherapy. No statistically significant difference in progression-free survival (PFS) between the two arms was observed (Bellmunt et al., N Engl J Med (2017); 376(11): 1015-26). EU approval for the same indication was granted in September 2017 and Japanese approval in January 2018. Other programmed cell death protein 1 (PD-1) and PD-L1 inhibitors are currently being evaluated in clinical trials for urothelial cancer, as first and second-line therapy (Mullane et al., Curr Opin Urol (2016); 26(6): 556-63).

While CPIs offer a new approach to treatment of metastatic urothelial cancer, tumor responses have occurred in a minority of patients and the improvement in long-term survival is only a few months. For example, in May 2017. Roche announced that a confirmatory phase 3 trial of second-line atezolizumab had failed to meet its primary endpoint of OS (Roche, press release “Roche provides update on phase III study of Tecentriq (atezolizumab) in people with previously treated advanced bladder cancer,” 10 May 2017). Most patients with locally advanced or metastatic urothelial cancer do not respond to CPIs and many who do respond ultimately develop disease progression (Rosenberg et al., Lancet (2016); 387(10031): 1909-20). Novel treatments are still needed, particularly for patients who have not responded to CPIs or who have progressed following CPI therapy.

Currently, no therapies are approved for patients previously treated with a CPI. Although taxanes are not approved in this setting, they are a common choice for third line treatment (and were a standard second-line treatment before atezolizumab was approved). Taxanes have response rates of approximately 10% as second-line therapy, with progression-free survival (PFS) and OS of only 3.3 months and 7.4 months, respectively (Bellmunt et al., N Engl J Med (2017):376(11): 1015-26). No data are currently available regarding the clinical activity of taxanes in the third line setting after CPI therapy.

The lack of approved therapies for patients with metastatic urothelial cancer after treatment with a CPI and the limited activity observed with second-line chemotherapy adequately demonstrate that this population has significant unmet medical need.

Bladder Cancer

Of all new cases of cancer in the United States, bladder cancer represents approximately 5 percent in men (fifth most common neoplasm) and 3 percent in women (eighth most common neoplasm). The incidence is increasing slowly, concurrent with an increasing older population. American Cancer Society (cancer.org) estimates that there are 81,400 new cases annually, including 62,100 in men and 19,300 in women, which accounts for 4.5% of all cancer cases. The age-adjusted incidence in the United States is 20 per 100,000 for men and women. There are an estimated 17,980 deaths from bladder cancer in annually (13,050 in men and 4,930 in women), which accounts for 3% of cancer related deaths. Bladder cancer incidence and mortality strongly increase with age and will be an increasing problem as the population becomes more elderly. Globally, approximately 580,000 people will be diagnosed with bladder cancer in 2020, and bladder cancer will be attributed to approximately 210,000 deaths worldwide.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

There is a significant need for additional therapeutic methods for urothelial and bladder cancers. These include the use of antibodies and antibody drug conjugates as treatment modalities.

### 3. SUMMARY

Provided herein are methods for the treatment of various cancers in human subjects, including subjects with previously treated locally advanced or metastatic urothelial cancer, using an antibody drug conjugate (ADC) that binds 191P4D12.

In certain embodiments, the previous treatment includes an immune checkpoint inhibitor (CPI). In other embodiments, the human subject treated with the methods provided herein is ineligible to receive cisplatin treatment. In other embodiments, the human subject treated with the methods provided herein is ineligible to receive cisplatin treatment and has received previous treatment with a CPI (e.g., a PD1 or PDL1 inhibitor).
Embodiment 1. A method of preventing or treating cancer in a human subject, comprising administering to the subject an effective amount of an antibody drug conjugate,
wherein the antibody drug conjugate comprises an antibody or antigen binding fragment thereof that binds to 191P4D12 conjugated to one or more units of monomethyl auristatin E (MMAE);
wherein the subject has urothelial or bladder cancer;
wherein the subject has received an immune checkpoint inhibitor (CPI) therapy;
wherein the subject is ineligible to receive cisplatin treatment (cisplatin ineligible).
Embodiment 2. The method of embodiment 1, wherein the cisplatin ineligible subject is a platinum-naïve subject.
Embodiment 3. The method of embodiment 1 or 2, wherein the platinum-naïve subject is a subject that received platinum in the adjuvant or neoadjuvant setting and did not progress within 12 months of completion of the platinum treatment.
Embodiment 4. The method of embodiment 1 or 2, wherein the platinum-naïve subject is a subject that has not received prior platinum-containing or other chemotherapy in the locally advanced or metastatic setting.
Embodiment 5. The method of any one of embodiments 1 to 4, wherein the cisplatin ineligible subject has one or more of the conditions selected from the group consisting of: ECOG performance status score of 2, impaired renal function, and no less than Grade 2 hearing loss.
Embodiment 6. The method of embodiment 5, wherein the impaired renal function is determined by creatinine clearance (CrCl) less than 60 mL/min.
Embodiment 7. The method of embodiment 5, wherein the impaired renal function is determined by CrCl less than 60 but no less than 30 mL/min.
Embodiment 8. The method of embodiment 5, wherein the impaired renal function is determined by CrCl less than 30 but no less than 15 mL % min.
Embodiment 9. The method of any one of embodiments 1 to 8, wherein the cisplatin ineligible subject had progression or recurrence of the cancer during or following most recent therapy.
Embodiment 10. The method of any one of embodiments 1 to 8, wherein the cisplatin ineligible subject had progression or recurrence of the cancer during or following the CPI therapy.
Embodiment 11. The method of any one of embodiments 1 to 10, wherein the subject has a primary site of tumor in the lower urinary tract.
Embodiment 12. The method of any one of embodiments 1 to 10, wherein the subject has a primary site of tumor in the upper urinary tract
Embodiment 13. The method of any one of embodiments 1 to 12, wherein the subject has visceral metastases.
Embodiment 14. The method of any one of embodiments 1 to 13, wherein the subject has liver metastases.
Embodiment 15. The method of any one of embodiments 1 to 14, wherein the subject has at least 1 Bellmunt risk factor.
Embodiment 16. The method of any one of embodiments 1 to 15, wherein the subject has one or more of the conditions selected from the group consisting of:
   (i) absolute neutrophil count no less than 1.0×10⁹/L,
   (ii) platelet count no less than 100×10⁹/L,
   (iii) hemoglobin no less than 9 g/dL,
   (iv) serum bilirubin no more than either of 1.5 times of upper limit of normal (ULN) or 3 times ULN for patients with Gilbert's disease,
   (v) CrCl no less than 30 mL/min, and
   (vi) alanine aminotransferase and aspartate aminotransferase no more than 3 fold of ULN.
Embodiment 17. The method of embodiment 16, wherein the subject has all of conditions (i) to (vi) of embodiment 16.
Embodiment 18. The method of any one of embodiments 6 to 8, 16 and 17, wherein the CrCl is measured by 24 hour urine collection or estimated by the Cockcroft-Gault criteria.
Embodiment 19. The method of any one of embodiments 1 to 18, wherein the subject has no more than Grade 2 sensory or motor neuropathy.
Embodiment 20. The method of any one of embodiments 1 to 19, wherein the subject has no active central nervous system metastases.
Embodiment 21. The method of any one of embodiments 1 to 20, wherein the subject has no uncontrolled diabetes.
Embodiment 22. The method of embodiment 21, wherein the uncontrolled diabetes is determined by hemoglobin A1c (HbA1c) no less than 8% or HbA1c between 7 and 8% with associated diabetes symptoms that are not otherwise explained.
Embodiment 23. The method of embodiment 22, wherein the associated diabetes symptoms comprise or consist of polyuria, polydipsia, or both polyuria and polydipsia.
Embodiment 24. The method of any one of embodiments 1 to 23, wherein the subject has locally advanced or metastatic urothelial cancer.
Embodiment 25. The method of any one of embodiments 1 to 23, wherein the subject has locally advanced or metastatic bladder cancer.
Embodiment 26. The method of any one of embodiments 1 to 25, wherein the CPI therapy is a therapy of programmed death receptor-1 (PD-1) inhibitor.
Embodiment 27. The method of any one of embodiments 1 to 25, wherein the CPI therapy is a therapy of programmed death-ligand 1 (PD-L1) inhibitor.
Embodiment 28. The method of embodiment 26, wherein PD-1 inhibitor is nivolumab or pembrolizumab.
Embodiment 29. The method of embodiment 27, wherein PD-L1 inhibitor is selected from a group consisting of atezolizumab, avelumab, and durvalumab.
Embodiment 30. The method of any one of embodiments 1 to 29, wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.
Embodiment 31. The method of any one of embodiments 1 to 30, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO: 13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.
Embodiment 32. The method of any one of embodiments 1 to 30, wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO: 16, CDR-H2 consisting of the amino acid sequence of SEQ ID NO:17, CDR-H3 consisting of the amino acid sequence of SEQ ID NO:18 CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.
Embodiment 33. The method of any one of embodiments 1 to 32, wherein the antibody or antigen binding fragment thereof comprises a heavy chain variable region comprising the amino acid sequence of SEQ ID NO:22 and a light chain variable region comprising the amino acid sequence of SEQ ID NO:23.
Embodiment 34. The method of any one of embodiments 1 to 33, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.
Embodiment 35. The method of any one of embodiments 1 to 33, wherein the antigen binding fragment is an Fab, F(ab′)2, Fv or scFv.
Embodiment 36. The method of any one of embodiments 1 to 34, wherein the antibody is a fully human antibody.
Embodiment 37. The method of any one of embodiments 1 to 34 and 36, wherein the antibody is an IgG1 and light chain is a kappa light chain
Embodiment 38. The method of any one of embodiments 1 to 37, wherein the antibody or antigen binding fragment thereof is recombinantly produced.
Embodiment 39. The method of any one of embodiments 1 to 38, wherein the antibody or antigen binding fragment is conjugated to each unit of MMAE via a linker.
Embodiment 40. The method of embodiment 39, wherein the linker is an enzyme-cleavable linker, and wherein the linker forms a bond with a sulfur atom of the antibody or antigen binding fragment thereof.
Embodiment 41. The method of embodiment 39 or 40, wherein the linker has a formula of: -Aa-Ww-Yy-; wherein -A- is a stretcher unit, a is 0 or 1; -W- is an amino acid unit, w is an integer ranging from 0 to 12, and -Y- is a spacer unit, y is 0, 1, or 2.
Embodiment 42. The method of embodiment 41, wherein the stretcher unit has the structure of Formula (1) below; the amino acid unit is valine-citrulline; and the spacer unit is a PAB group comprising the structure of Formula (2) below:

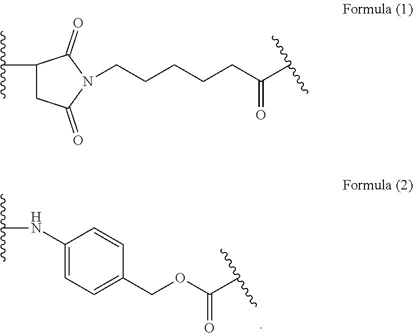 [SMILES: *C(=O)CCCCCN1C(=O)CC(*)C1=O ; *Nc1ccc(COC(*)=O)cc1]

Embodiment 43. The method of embodiment 41 or 42, wherein the stretcher unit forms a bond with a sulfur atom of the antibody or antigen binding fragment thereof, and wherein the spacer unit is linked to MMAE via a carbamate group.
Embodiment 44. The method of any one of embodiments 1 to 43, wherein the ADC comprises from 1 to 20 units of MMAE per antibody or antigen binding fragment thereof.
Embodiment 45. The method of any one of embodiments 1 to 44, wherein the ADC comprises from 1 to 10 units of MMAE per antibody or antigen binding fragment thereof.
Embodiment 46. The method of any one of embodiments 1 to 45, wherein the ADC comprises from 2 to 8 units of MMAE per antibody or antigen binding fragment thereof.
Embodiment 47. The method of any one of embodiments 1 to 46, wherein the ADC comprises from 3 to 5 units of MMAE per antibody or antigen binding fragment thereof.
Embodiment 48. The method of any one of embodiments 1 to 45, wherein the ADC has the following structure.

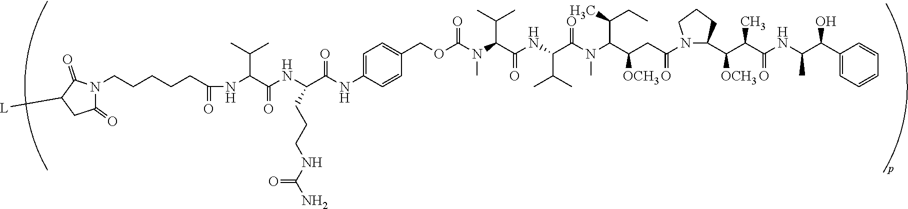

wherein L- represents the antibody or antigen binding fragment thereof and p is from 1 to 10.
Embodiment 49. The method of embodiment 48, wherein p is from 2 to 8.
Embodiment 50. The method of embodiment 48 or 49, wherein p is from 3 to 5.
Embodiment 51. The method of any one of embodiments 48 to 50, wherein p is from 3 to 4.
Embodiment 52. The method of any one of embodiments 48 to 51, wherein p is about 4.
Embodiment 53. The method of any one of embodiments 48 to 51, wherein the average p value of the effective amount of the antibody drug conjugates is about 3.8.
Embodiment 54. The method of any one of embodiments 1 to 53, wherein the ADC is administered at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.
Embodiment 55. The method of any one of embodiments 1 to 54, wherein the ADC is administered at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.
Embodiment 56. The method of any one of embodiments 1 to 55, wherein the ADC is administered at a dose of about 1 mg/kg of the subject's body weight.
Embodiment 57. The method of any one of embodiments 1 to 55, wherein the ADC is administered at a dose of about 1.25 mg/kg of the subject's body weight.
Embodiment 58. The method of any one of embodiments 1 to 57, wherein the ADC is administered by an intravenous (IV) injection or infusion.
Embodiment 59. The method of any one of embodiments 1 to 58, wherein the ADC is administered by an IV injection or infusion three times every four-week cycle.
Embodiment 60. The method of any one of embodiments 1 to 59, wherein the ADC is administered by an IV injection or infusion on Days 1, 8 and 15 of every four-week cycle.
Embodiment 61. The method of any one of embodiments 1 to 60, wherein the ADC is administered by an IV injection or infusion over about 30 minutes three times every four-week cycle.
Embodiment 62. The method of any one of embodiments 1 to 61, wherein the ADC is administered by an IV injection or infusion over about 30 minutes on Days 1, 8 and 15 of every four-week cycle.
Embodiment 63. The method of any one of embodiments 1 to 62, wherein the ADC is formulated in a pharmaceutical composition comprising L-histidine, polysorbate-20 (TWEEN-20), and trehalose dehydrate.
Embodiment 64. The method of any one of embodiments 1 to 63, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.
Embodiment 65. The method of any one of embodiments 1 to 63, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.
Embodiment 66. The method of any one of embodiments 1 to 65, wherein the ADC has the following structure:

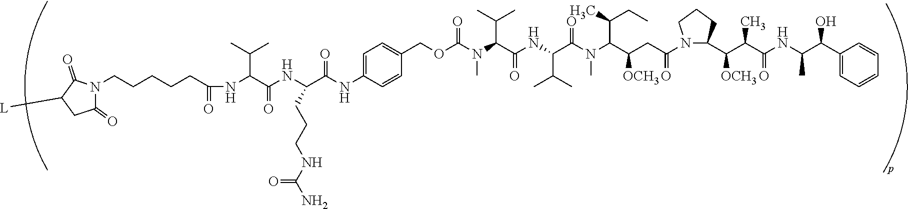

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on Days 1, 8 and 15 of every four-week cycle.
Embodiment 67. The method of any one of embodiments 1 to 66, whereby the subject has a complete response following the treatment.
Embodiment 68. The method of any one of embodiments 1 to 66, wherein the subject has a partial response following the treatment.
Embodiment 69. The method of any one of embodiments 1 to 66, wherein the subject has a complete response or a partial response following the treatment.
Embodiment 70. The method of any one of embodiments 1 to 66, wherein the subject has a stable disease following the treatment.
Embodiment 71. The method of any one of embodiments 1 to 66, wherein the subject has a duration of response of at least or about 10 months following the treatment.
Embodiment 72. The method of any one of embodiments 1 to 66, wherein the subject has a duration of response ranging from 5 to 22 months following the treatment.
Embodiment 73. The method of any one of embodiments 1 to 66, wherein the subject has a progression free survival of at least or about 5 months following the treatment.
Embodiment 74. The method of any one of embodiments 1 to 66, wherein the subject has a progression free survival ranging from 5 to 9 months following the treatment.
Embodiment 75. The method of any one of embodiments 1 to 66, wherein the subject has an overall survival of at least or about 14 months following the treatment.
Embodiment 76. The method of any one of embodiments 1 to 66, wherein the subject has an overall survival ranging from 10 to 19 months following the treatment.
Embodiment 77. The method of any one of embodiments 1 to 66, wherein a population of the subjects is treated by the methods, and wherein percentage of the subjects having complete response in the treated population is at least or about 20%.
Embodiment 78. The method of any one of embodiments 1 to 66, wherein a population of the subjects is treated by the methods, and wherein percentage of the subjects having partial response in the treated population is at least or about 31%.
Embodiment 79. The method of any one of embodiments 1 to 66, wherein a population of the subjects is treated by the methods, and wherein objective response rate in the treated population is at least or about 51%.
Embodiment 80. The method of any one of embodiments 1 to 66, wherein a population of the subjects is treated by the methods, and wherein objective response rate in the treated population ranges from 40% to 63%.
Embodiment 81. The method of any one of embodiments 1 to 66, wherein a population of the subjects is treated by the methods, and wherein percentage of the subjects having stable disease in the treated population is at least or about 30%.
Embodiment 82. The method of any one of embodiments 1 to 66, wherein a population of the subjects is treated by the methods, and wherein median duration of response in the treated population is at least or about 10 months.
Embodiment 83. The method of any one of embodiments 1 to 66, wherein a population of the subjects is treated by the methods, and wherein duration of response in the treated population ranges from 5 to 22 months.
Embodiment 84. The method of any one of embodiments 1 to 66, wherein a population of the subjects is treated by the methods, and wherein median progression free survival in the treated population is at least or about 5 months.
Embodiment 85. The method of any one of embodiments 1 to 66, wherein a population of the subjects is treated by the methods, and wherein progression free survival in the treated population ranges from 5 to 9 months.
Embodiment 86. The method of any one of embodiments 1 to 66, wherein a population of the subjects is treated by the methods, and wherein median overall survival in the treated population is at least or about 14 months.
Embodiment 87. The method of any one of embodiments 1 to 66, wherein a population of the subjects is treated by the methods, and wherein overall survival in the treated population ranges from 10 to 19 months.
Embodiment 88. The method of any one of embodiments 1 to 67 and 69, wherein the complete response rate is at least or about 20% for a population of subjects treated with the method.
Embodiment 89. The method of any one of embodiments 1 to 66, 68, and 69, wherein the partial response rate is at least or about 31% for a population of subjects treated with the method.
Embodiment 90. The method of any one of embodiments 1 to 69, wherein objective response rate is at least or about 51% for a population of subjects treated with the method.
Embodiment 91. The method of any one of embodiments 1 to 69, wherein objective response rate is from 40% to 63% for a population of subjects treated with the method.
Embodiment 92. The method of any one of embodiments 1 to 66 and 70, wherein the stable disease rate is at least or about 30% for a population of subjects treated with the method.
Embodiment 93. The method of any one of embodiments 1 to 66, 71 and 72, wherein the median duration of response is at least or about 10 months for a population of subjects treated with the method.
Embodiment 94. The method of any one of embodiments 1 to 66, 71 and 72, wherein the duration of response is from 5 to 22 months for a population of subjects treated with the method.
Embodiment 95. The method of any one of embodiments 1 to 66, 73 and 74, wherein the median progression free survival is at least or about 5 months for a population of subjects treated with the method.
Embodiment 96. The method of any one of embodiments 1 to 66, 73 and 74, wherein the progression free survival is from 5 to 9 months for a population of subjects treated with the method.
Embodiment 97. The method of any one of embodiments 1 to 66, 75 and 76, wherein the median overall survival is at least or about 14 months for a population of subjects treated with the method.
Embodiment 98. The method of any one of embodiments 1 to 66, 75 and 76, wherein the overall survival is from 10 to 19 months for a population of subjects treated with the method.

### 4. BRIEF DESCRIPTION OF THE DRAWINGS

FIGS. 1A-1E depict the nucleotide and amino acid sequences of nectin-4 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2 depicts the overall study design of the clinical study described in Section 6.1.

FIG. 3 depicts a study schema of the clinical study, which is a single-arm, open-label two-cohort study in metastatic urothelial cancer, as described in Section 6.1.

FIG. 4 depicts objective response rate (ORR) in the clinical study described in Section 6.1.

FIG. 5 depicts ORR subgroup analysis in the clinical study described in Section 6.1.

FIG. 6 depicts duration of response per blinded independent central review in the clinical study described in Section 6.1.

FIG. 7 depicts time to response and duration of response per central review in the clinical study described in Section 6.1.

FIG. 8 depicts progression-free survival per blinded independent central review in the clinical study described in Section 6.1.

FIG. 9 depicts overall survival in the clinical study described in Section 6.1.

FIG. 10 depicts Nectin-4 distribution between responders and non-responders per blinded independent central review in the clinical study described in Section 6.1.

FIG. 11. depicts duration of response (DOR) per blinded independent central review in the clinical study described in Section 6.1.

FIG. 12 depicts ORR subgroup analysis per blinded independent central review in the clinical study described in Section 6.1.

FIG. 13 depicts progression-free survival per blinded independent central review in the clinical study described in Section 6.1.

FIG. 14 depicts overall survival in the clinical study described in Section 6.1.

### 5. DETAILED DESCRIPTION

Before the present disclosure is further described, it is to be understood that the disclosure is not limited to the particular embodiments set forth herein, and it is also to be understood that the terminology used herein is for describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010), and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See. e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see. e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See. e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet®QK384 system, or by Biacore®, using, for example, a Biacore® TM-2000 or a Biacore® TM-3000. An “on-rate” or “rate of association” or “association rate” or “kon” may also be determined with the same biolayer interferometry (BLI) or surface plasmon resonance (SPR) techniques described above using, for example, the Octet®QK384, the Biacore® TM-2000, or the Biacore®TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96, Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see. e.g., Jakobovits, 1995, Curr. Opin. Biotechnol. 6(5):561-66; Brüggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies. e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see. e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See. e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see. e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia. Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size. α, δ, and γ contain approximately 450 amino acids, while μ and ε contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see. e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun. 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below in Table 1

TABLE 1

| | Kabat | AbM | Chothia | Contact | IMGT |
|---|---|---|---|---|---|
| CDR-L1 | L24--L34 | L24--L34 | L24--L34 | L30--L36 | L27--L38 |
| CDR-L2 | L50--L56 | L50--L56 | L50--L56 | L46--L55 | L56--L65 |
| CDR-L3 | L89--L97 | L89--L97 | L89--L97 | L89--L96 | L105-L117 |
| CDR-H1 | H31--H35B | H26--H35B | H26--H32 . . . 34 | H30--H35B | H27--H38 |
| | (Kabat | | | | |
| | Numbering) | | | | |
| CDR-H1 | H31--H35 | H26--H35 | H26--H32 | H30--H35 | |
| | (Chothia | | | | |
| | Numbering) | | | | |
| CDR-H2 | H50--H65 | H50--H58 | H52--H56 | H47--H58 | H56--H65 |
| CDR-H3 | H95--H102 | H95--H102 | H95--H102 | H93--H101 | H105-H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise indicated by context, a hyphen (-) designates the point of attachment to the pendant molecule.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see. e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University): Henikoff et al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE | THREE | |
|---|---|---|
| LETTER | LETTER | FULL NAME |
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid substitution
matrix (block substitution matrix). The higher the value, the more likely
a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, MD (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE: Databases=non-redundant, GenBank+EMBL+DDBJ+PDB+GenBank CDS translations+SwissProtein+SPupdate+PIR Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul. 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see. e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

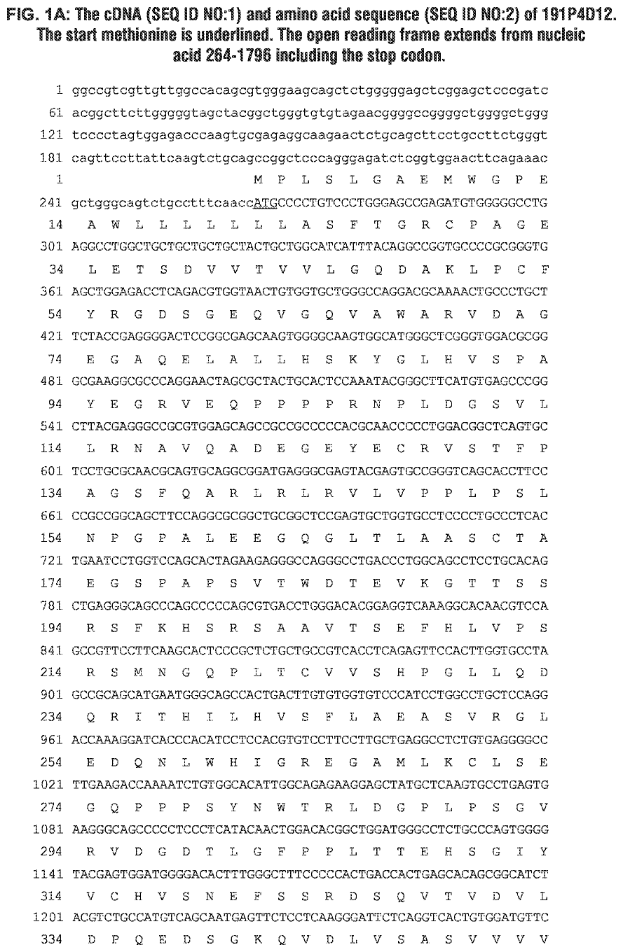

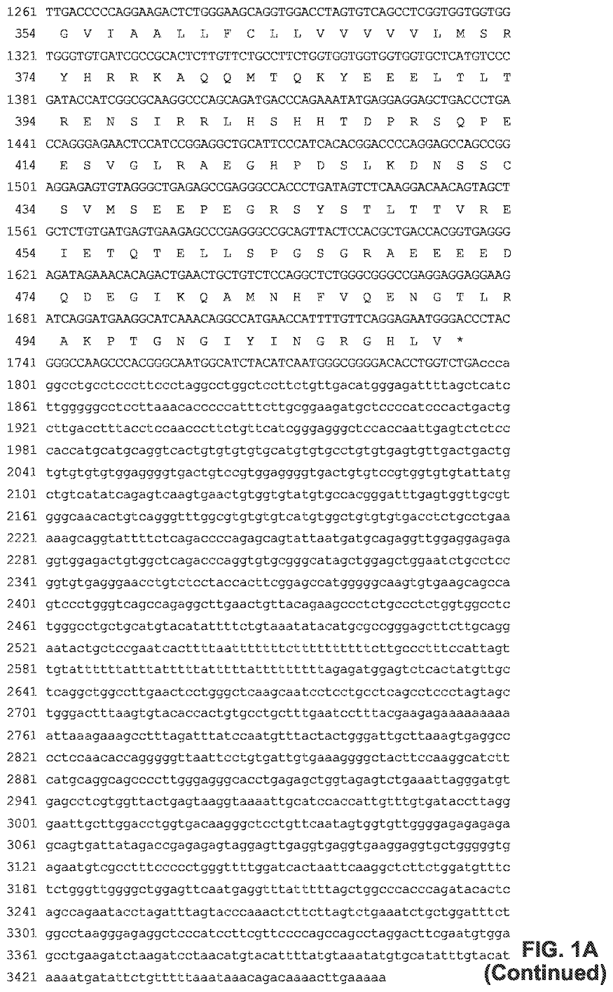

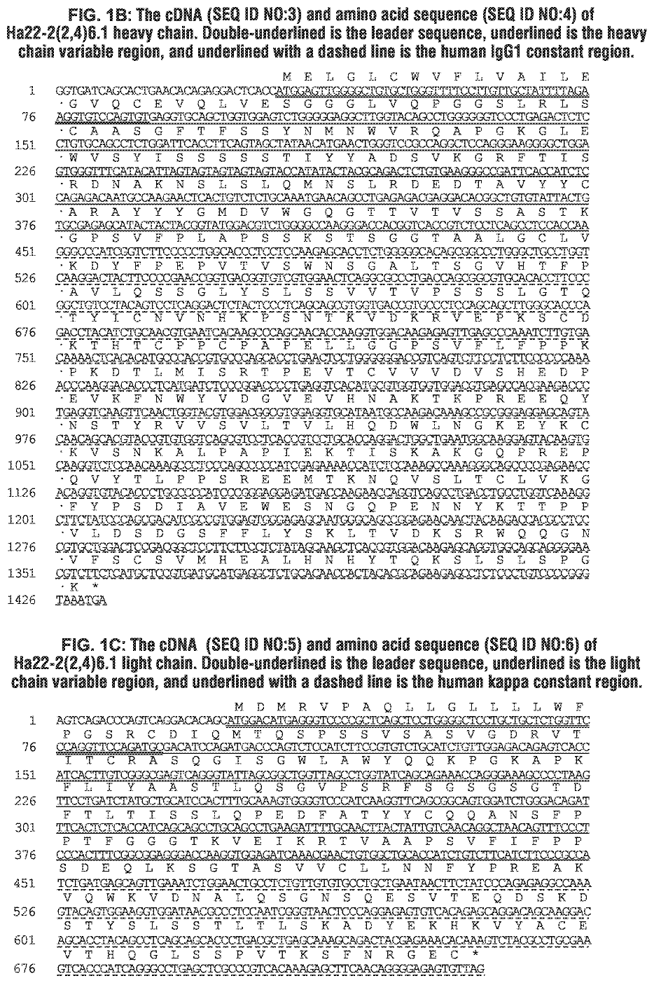

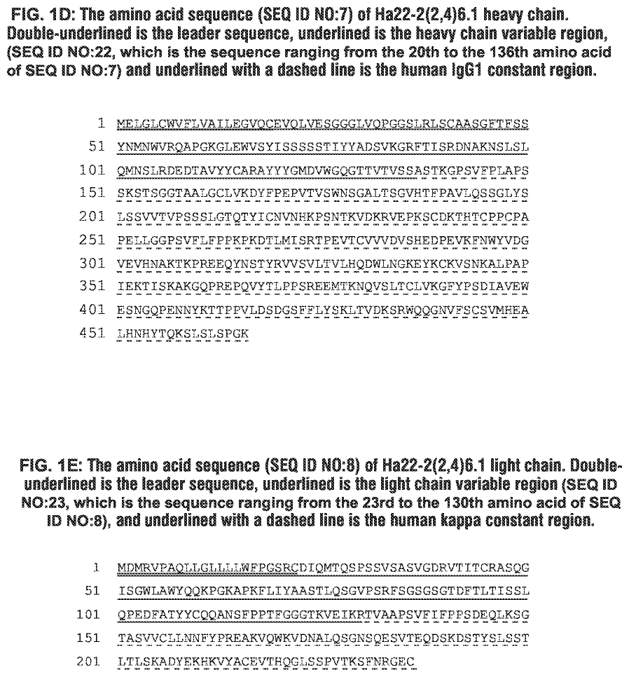

The term “variant” refers to a molecule that exhibits a variation from a described type or norm, such as a protein that has one or more different amino acid residues in the corresponding position(s) of a specifically described protein (e.g. the 191P4D12 protein shown in FIG. 1A.) An analog is an example of a variant protein. Splice isoforms and single nucleotides polymorphisms (SNPs) are further examples of variants.

The “191P4D12 proteins” and/or “191P4D12 related proteins” of the disclosure include those specifically identified herein (see, FIG. 1A), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the disclosure, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids. The term “191P4D12” is used interchangeably with nectin-4.

### 5.2 Methods of Treating Cancer

Urothelial cancer and bladder cancer (including locally advanced urothelial cancer, metastatic urothelial cancer, locally advanced bladder cancer and metastatic bladder cancer), in patients who have received immunotherapy and are ineligible for cisplatin are particularly difficult disease to treat. Typically, these patients are frail, suffer from multiple comorbidities beyond their urothelial cancer/bladder cancer and are not able to tolerate additional treatment beyond immunotherapy, leading many to discontinue therapy altogether. As such, these patients have a poor prognosis and few treatment options. This disclosure is based in part upon the results of the first clinical trial to demonstrate objective responses in such patients. The disclosure thus provides demonstrated efficacious methods to treat patients with urothelial cancer and/or bladder cancer (including locally advanced urothelial cancer, metastatic urothelial cancer, locally advanced bladder cancer and metastatic bladder cancer) who have previously received immunotherapy but are ineligible for cisplatin in this setting due to inadequate kidney function or other conditions as provided herein. Prior to the results described herein, there was considerable uncertainty whether the methods which are provided herein would be efficacious given that this patient population has historically proven so difficult to treat. As described further below, the level of efficacy obtained was particularly notable and surprising.

5.2.1 Methods of Treating Cancer in General and for Selected Patients

Provided herein are methods for the treatment of various cancers in subjects, including subjects with previously treated locally advanced or metastatic urothelial cancer, using an antibody drug conjugate (ADC) that binds 191P4D12.

In one aspect, provided herein are methods for the treatment of cancer in a subject using an ADC that binds 191P4D12. In some embodiments, the human subject treated with the methods provided herein has received previous cancer treatment other than the ADC that binds 191P4D12. In certain embodiments, the previous treatment includes or consists of an immune checkpoint inhibitor (CPI). In some embodiments, the human subject treated with the methods provided herein is ineligible to receive cisplatin treatment. In other embodiments, the human subject treated with the methods provided herein is ineligible to receive cisplatin treatment and has received previous treatment including or consisting of a CPI. In certain embodiments, the human subject treated with the methods provided herein is ineligible to receive cisplatin treatment, has received previous treatment including or consisting of a CPI, and is a platinum-naïve human subject. In some further embodiments, the human subject treated with the methods provided herein is ineligible to receive cisplatin treatment, has received previous treatment including or consisting of a CPI, and is a human subject that received platinum in the adjuvant or neoadjuvant setting and did not progress within 12 months of completion of the platinum treatment. In still further embodiments, the human subject treated with the methods provided herein is ineligible to receive cisplatin treatment, has received previous treatment including or consisting of a CPI, and is a human subject that has not received prior platinum-containing or other chemotherapy in the locally advanced or metastatic setting. In some embodiments, the cancer is urothelial cancer. In certain embodiments, the cancer is bladder cancer. In one embodiment, the cancer is locally advanced cancer. In another embodiment, the cancer is metastatic cancer. In a further embodiment, the cancer is locally advanced urothelial cancer. In yet another embodiment, the cancer is metastatic urothelial cancer. In one embodiment, the cancer is locally advanced bladder cancer. In another embodiment, the cancer is metastatic bladder cancer. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In another aspect, provided herein are methods for the treatment of urothelial cancer in a human subject, comprising administering to the human subject an effective amount of an ADC comprising an antibody or antigen binding fragment thereof that binds to 191P4D12. In one embodiment of the methods for the treatment of urothelial cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of urothelial cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of urothelial cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of urothelial cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of urothelial cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In a further aspect, provided herein are methods for the treatment of locally advanced urothelial cancer in a human subject, comprising administering to the human subject an effective amount of an ADC comprising an antibody or antigen binding fragment thereof that binds to 191P4D12. In one embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In yet another aspect, provided herein are methods for the treatment of metastatic urothelial cancer in a human subject, comprising administering to the human subject an effective amount of an ADC comprising an antibody or antigen binding fragment thereof that binds to 191P4D12. In one embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In still another aspect, provided herein are methods for the treatment of bladder cancer in a human subject, comprising administering to the human subject an effective amount of an ADC comprising an antibody or antigen binding fragment thereof that binds to 191P4D12. In one embodiment of the methods for the treatment of bladder cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of bladder cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of bladder cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of bladder cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of bladder cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In one aspect, provided herein are methods for the treatment of locally advanced bladder cancer in a human subject, comprising administering to the human subject an effective amount of an ADC comprising an antibody or antigen binding fragment thereof that binds to 191P4D12. In one embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In another aspect, provided herein are methods for the treatment of metastatic bladder cancer in a human subject, comprising administering to the human subject an effective amount of an ADC comprising an antibody or antigen binding fragment thereof that binds to 191P4D12. In one embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy.

In a further aspect, provided herein are methods for the treatment of cancer in a human subject, comprising administering to the human subject an effective amount of an ADC comprising an antibody or antigen binding fragment thereof that binds to 191P4D12. In one embodiment of the methods for the treatment of cancer provided herein, the method comprises administering to the human subject an effective amount of an ADC comprising an antibody or antigen binding fragment thereof that binds to 191P4D12, wherein the human subject has received a CPI therapy. In another embodiment of the methods for the treatment of cancer provided herein, the method comprises administering to the human subject an effective amount of an ADC comprising an antibody or antigen binding fragment thereof that binds to 191P4D12, wherein the human subject has received a CPI therapy and the human subject is ineligible to receive cisplatin treatment. In a further embodiment of the methods for the treatment of cancer provided herein, the method comprises administering to the human subject an effective amount of an ADC comprising an antibody or antigen binding fragment thereof that binds to 191P4D12, wherein the human subject has received a CPI therapy, the human subject is ineligible to receive cisplatin treatment, and wherein the human subject is a platinum-naïve subject. In yet another embodiment of the methods for the treatment of cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject has not received prior platinum-containing or other chemotherapy in the locally advanced or metastatic setting. In still a further embodiment of the methods for the treatment of cancer provided herein, the method comprises administering to the human subject an effective amount of an ADC comprising an antibody or antigen binding fragment thereof that binds to 191P4D12, wherein the human subject has received a CPI therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the CPI therapy. In some embodiments of the methods provided herein, including in this paragraph, the cancer is locally advanced cancer. In certain embodiments, of the methods provided herein, including in this paragraph, the cancer is metastatic cancer.

In one aspect, provided herein are methods for the treatment of cancer in a subject using an ADC that binds 191P4D12, wherein the human subject treated with the methods provided herein has received a previous PD-1 inhibitor therapy. In some embodiments, the human subject treated with the methods provided herein is ineligible to receive cisplatin treatment. In other embodiments, the human subject treated with the methods provided herein is ineligible to receive cisplatin treatment and has received previous treatment including or consisting of a PD-1 inhibitor. In certain embodiments, the human subject treated with the methods provided herein is ineligible to receive cisplatin treatment, has received previous treatment including or consisting of a PD-1 inhibitor, and is a platinum-naïve human subject. In some further embodiments, the human subject treated with the methods provided herein is ineligible to receive cisplatin treatment, has received previous treatment including or consisting of a PD-1 inhibitor, and is a human subject that received platinum in the adjuvant or neoadjuvant setting and did not progress within 12 months of completion of the platinum treatment. In still further embodiments, the human subject treated with the methods provided herein is ineligible to receive cisplatin treatment, has received previous treatment including or consisting of a PD-1 inhibitor, and is a human subject that has not received prior platinum-containing or other chemotherapy in the locally advanced or metastatic setting. In some embodiments, the cancer is urothelial cancer. In certain embodiments, the cancer is bladder cancer. In one embodiment, the cancer is locally advanced cancer. In another embodiment, the cancer is metastatic cancer. In a further embodiment, the cancer is locally advanced urothelial cancer. In yet another embodiment, the cancer is metastatic urothelial cancer. In one embodiment, the cancer is locally advanced bladder cancer. In another embodiment, the cancer is metastatic bladder cancer. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In another aspect, provided herein are methods for the treatment of urothelial cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In a further aspect, provided herein are methods for the treatment of locally advanced urothelial cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In yet another aspect, provided herein are methods for the treatment of metastatic urothelial cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In still another aspect, provided herein are methods for the treatment of bladder cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In one aspect, provided herein are methods for the treatment of locally advanced bladder cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In another aspect, provided herein are methods for the treatment of metastatic bladder cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy.

In a further aspect, provided herein are methods for the treatment of cancer in a human subject, comprising administering to the human subject an effective amount of an ADC comprising an antibody or antigen binding fragment thereof that binds to 191P4D12, wherein the human subject has received a PD-1 inhibitor therapy. In another embodiment of the methods for the treatment of cancer provided herein, the method comprises administering to the human subject an effective amount of an ADC comprising an antibody or antigen binding fragment thereof that binds to 191P4D12, wherein the human subject has received a PD-1 inhibitor therapy and the human subject is ineligible to receive cisplatin treatment. In a further embodiment of the methods for the treatment of cancer provided herein, the method comprises administering to the human subject an effective amount of an ADC comprising an antibody or antigen binding fragment thereof that binds to 191P4D12, wherein the human subject has received a PD-1 inhibitor therapy, the human subject is ineligible to receive cisplatin treatment, and wherein the human subject is a platinum-naïve subject. In yet another embodiment of the methods for the treatment of cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of cancer provided herein, the method comprises administering to the human subject an effective amount of an ADC comprising an antibody or antigen binding fragment thereof that binds to 191P4D12, wherein the human subject has received a PD-1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-1 inhibitor therapy. In some embodiments of the methods provided herein, including in this paragraph, the cancer is locally advanced cancer. In certain embodiments, of the methods provided herein, including in this paragraph, the cancer is metastatic cancer.

In one aspect, provided herein are methods for the treatment of cancer in a subject using an ADC that binds 191P4D12, wherein the human subject treated with the methods provided herein has received a previous PD-L1 inhibitor therapy. In some embodiments, the human subject treated with the methods provided herein is ineligible to receive cisplatin treatment. In other embodiments, the human subject treated with the methods provided herein is ineligible to receive cisplatin treatment and has received previous treatment including or consisting of a PD-L1 inhibitor. In certain embodiments, the human subject treated with the methods provided herein is ineligible to receive cisplatin treatment, has received previous treatment including or consisting of a PD-L1 inhibitor, and is a platinum-naïve human subject. In some further embodiments, the human subject treated with the methods provided herein is ineligible to receive cisplatin treatment, has received previous treatment including or consisting of a PD-L1 inhibitor, and is a human subject that received platinum in the adjuvant or neoadjuvant setting and did not progress within 12 months of completion of the platinum treatment. In still further embodiments, the human subject treated with the methods provided herein is ineligible to receive cisplatin treatment, has received previous treatment including or consisting of a PD-L1 inhibitor, and is a human subject that has not received prior platinum-containing or other chemotherapy in the locally advanced or metastatic setting. In some embodiments, the cancer is urothelial cancer. In certain embodiments, the cancer is bladder cancer. In one embodiment, the cancer is locally advanced cancer. In another embodiment, the cancer is metastatic cancer. In a further embodiment, the cancer is locally advanced urothelial cancer. In yet another embodiment, the cancer is metastatic urothelial cancer. In one embodiment, the cancer is locally advanced bladder cancer. In another embodiment, the cancer is metastatic bladder cancer. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In another aspect, provided herein are methods for the treatment of urothelial cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of urothelial cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In a further aspect, provided herein are methods for the treatment of locally advanced urothelial cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of locally advanced urothelial cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In yet another aspect, provided herein are methods for the treatment of metastatic urothelial cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of metastatic urothelial cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In still another aspect, provided herein are methods for the treatment of bladder cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of bladder cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In one aspect, provided herein are methods for the treatment of locally advanced bladder cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of locally advanced bladder cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In another aspect, provided herein are methods for the treatment of metastatic bladder cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of metastatic bladder cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy.

In a further aspect, provided herein are methods for the treatment of cancer in a human subject, comprising administering to the human subject an effective amount of an ADC comprising an antibody or antigen binding fragment thereof that binds to 191P4D12, wherein the human subject has received a PD-L1 inhibitor therapy. In another embodiment of the methods for the treatment of cancer provided herein, the method comprises administering to the human subject an effective amount of an ADC comprising an antibody or antigen binding fragment thereof that binds to 191P4D12, wherein the human subject has received a PD-L1 inhibitor therapy and the human subject is ineligible to receive cisplatin treatment. In a further embodiment of the methods for the treatment of cancer provided herein, the method comprises administering to the human subject an effective amount of an ADC comprising an antibody or antigen binding fragment thereof that binds to 191P4D12, wherein the human subject has received a PD-L1 inhibitor therapy, the human subject is ineligible to receive cisplatin treatment, and wherein the human subject is a platinum-naïve subject. In yet another embodiment of the methods for the treatment of cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject has not received prior platinum-containing or other chemotherapy in the locally advanced or metastatic setting. In still a further embodiment of the methods for the treatment of cancer provided herein, the method comprises administering to the human subject an effective amount of an ADC comprising an antibody or antigen binding fragment thereof that binds to 191P4D12, wherein the human subject has received a PD-L1 inhibitor therapy, wherein the human subject is ineligible to receive cisplatin treatment, and wherein the human subject received platinum in the adjuvant or neoadjuvant setting and did not progress within 12 months of completion of the platinum treatment. In further embodiments of the methods provided herein, including those provided in this paragraph, the subject had progression or recurrence of the cancer during or following most recent therapy. In other embodiments of the methods provided herein, including those provided in this paragraph, the subject had progression or recurrence of the cancer during or following the PD-L1 inhibitor therapy. In some embodiments of the methods provided herein, including in this paragraph, the cancer is locally advanced cancer. In certain embodiments, of the methods provided herein, including in this paragraph, the cancer is metastatic cancer.

5.2.1.1 Cisplatin Ineligible Patients

Various conditions can be used to determine the cisplatin ineligibility for the human subjects for the methods provided herein, including but not limited to the methods of the preceding paragraphs. In one embodiment, the conditions for determining the cisplatin ineligibility comprise or consist of ECOG performance status score of 2. In some embodiments, the conditions for determining the cisplatin ineligibility comprise or consist of impaired renal function. In certain embodiments, the conditions for determining the cisplatin ineligibility comprise or consist of no less than Grade 2 hearing loss. In one embodiment, the conditions for determining the cisplatin ineligibility comprise or consist of ECOG performance status score of 2 and impaired renal function. In some embodiments, the conditions for determining the cisplatin ineligibility comprise or consist of ECOG performance status score of 2 and no less than Grade 2 hearing loss. In further embodiments, the conditions for determining the cisplatin ineligibility comprise or consist of impaired renal function and no less than Grade 2 hearing loss. In yet other embodiments, the conditions for determining the cisplatin ineligibility comprise or consist of ECOG performance status score of 2, impaired renal function, and no less than Grade 2 hearing loss. In some embodiments, the conditions for determining the cisplatin ineligibility comprise or consist of any one of ECOG performance status score of 2, impaired renal function, and no less than Grade 2 hearing loss. In some embodiments, the conditions for determining the cisplatin ineligibility comprise or consist of any two of ECOG performance status score of 2, impaired renal function, and no less than Grade 2 hearing loss, in any combination or permutation. In some embodiments, the conditions for determining the cisplatin ineligibility comprise or consist of all three of ECOG performance status score of 2, impaired renal function, and no less than Grade 2 hearing loss.

Impaired renal function can be determined as various means known and available in the art. Various embodiments are provided herein to determine the impaired renal function for the human subjects for the methods provided herein, including but not limited to the methods of the preceding paragraph. In one embodiment, the impaired renal function is determined by creatinine clearance (CrCl) less than 60 mL/min. In some embodiments, the impaired renal function is determined by CrCl less than 60 but no less than 30 mL/min. In certain embodiments, the impaired renal function is determined by CrCl less than 30 but no less than 15 mL/min. In some embodiments of the methods provided in this paragraph, the CrCl is measured by 24 hour urine collection. In other embodiments of the methods provided in this paragraph, the CrCl is estimated by the Cockcroft-Gault criteria.

As such, some specific conditions based on creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of CrCl less than 60 mL/min. In one embodiment, the conditions for determining the cisplatin ineligibility comprise or consist of ECOG performance status score of 2 and CrCl less than 60 mL/min. In further embodiments, the conditions for determining the cisplatin ineligibility comprise or consist of CrCl less than 60 mL/min and no less than Grade 2 hearing loss. In yet other embodiments, the conditions for determining the cisplatin ineligibility comprise or consist of ECOG performance status score of 2, CrCl less than 60 mL/min, and no less than Grade 2 hearing loss. In some embodiments, the conditions for determining the cisplatin ineligibility comprise or consist of any one of ECOG performance status score of 2. CrCl less than 60 mL/min, and no less than Grade 2 hearing loss. In some embodiments, the conditions for determining the cisplatin ineligibility comprise or consist of any two of ECOG performance status score of 2. CrCl less than 60 mL/min, and no less than Grade 2 hearing loss, in any combination or permutation. In some embodiments, the conditions for determining the cisplatin ineligibility comprise or consist of all three of ECOG performance status score of 2, CrCl less than 60 mL/min, and no less than Grade 2 hearing loss. In some embodiments of the methods provided in this paragraph, the CrCl is measured by 24 hour urine collection. In other embodiments of the methods provided in this paragraph, the CrCl is estimated by the Cockcroft-Gault criteria.

Alternatively, other specific conditions based on creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of CrCl less than 60 but no less than 30 mL/min. In one embodiment, the conditions for determining the cisplatin ineligibility comprise or consist of ECOG performance status score of 2 and CrCl less than 60 but no less than 30 mL/min. In further embodiments, the conditions for determining the cisplatin ineligibility comprise or consist of CrCl less than 60 but no less than 30 mL/min and no less than Grade 2 hearing loss. In yet other embodiments, the conditions for determining the cisplatin ineligibility comprise or consist of ECOG performance status score of 2, CrCl less than 60 but no less than 30 mL/min, and no less than Grade 2 hearing loss. In some embodiments, the conditions for determining the cisplatin ineligibility comprise or consist of any one of ECOG performance status score of 2, CrCl less than 60 but no less than 30 mL/min, and no less than Grade 2 hearing loss. In some embodiments, the conditions for determining the cisplatin ineligibility comprise or consist of any two of ECOG performance status score of 2, CrCl less than 60 but no less than 30 mL/min, and no less than Grade 2 hearing loss, in any combination or permutation. In some embodiments, the conditions for determining the cisplatin ineligibility comprise or consist of all three of ECOG performance status score of 2. CrCl less than 60 but no less than 30 mL/min, and no less than Grade 2 hearing loss. In some embodiments of the methods provided in this paragraph, the CrCl is measured by 24 hour urine collection. In other embodiments of the methods provided in this paragraph, the CrCl is estimated by the Cockcroft-Gault criteria.

Similarly, further specific conditions based on creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of CrCl less than 30 but no less than 15 mL/min. In one embodiment, the conditions for determining the cisplatin ineligibility comprise or consist of ECOG performance status score of 2 and CrCl less than 30 but no less than 15 mL/min. In further embodiments, the conditions for determining the cisplatin ineligibility comprise or consist of CrCl less than 30 but no less than 15 mL/min and no less than Grade 2 hearing loss. In yet other embodiments, the conditions for determining the cisplatin ineligibility comprise or consist of ECOG performance status score of 2, CrCl less than 30 but no less than 15 mL/min, and no less than Grade 2 hearing loss. In some embodiments, the conditions for determining the cisplatin ineligibility comprise or consist of any one of ECOG performance status score of 2, CrCl less than 30 but no less than 15 mL/min, and no less than Grade 2 hearing loss. In some embodiments, the conditions for determining the cisplatin ineligibility comprise or consist of any two of ECOG performance status score of 2, CrCl less than 30 but no less than 15 mL/min, and no less than Grade 2 hearing loss, in any combination or permutation. In some embodiments, the conditions for determining the cisplatin ineligibility comprise or consist of all three of ECOG performance status score of 2, CrCl less than 30 but no less than 15 mL/min, and no less than Grade 2 hearing loss. In some embodiments of the methods provided in this paragraph, the CrCl is measured by 24 hour urine collection. In other embodiments of the methods provided in this paragraph, the CrCl is estimated by the Cockcroft-Gault criteria.

5.2.1.2 Additional Patient Demographics

Additionally, the human subjects for whom the methods provided herein can be used are human subjects having various other conditions. In one embodiment, the human subjects for whom the methods provided herein can have a primary site of tumor in the lower urinary tract. In some embodiments, the human subjects for whom the methods provided herein can have visceral metastases. In certain embodiments, the human subjects for whom the methods provided herein can have liver metastases. In other embodiments, the human subjects for whom the methods provided herein can have at least 1 Bellmunt risk factor. In yet other embodiments, the human subjects for whom the methods provided herein can have ECOG performance status score of 0. In one embodiment, the human subjects for whom the methods provided herein can have a primary site of tumor in the lower urinary tract and visceral metastases. In some embodiments, the human subjects for whom the methods provided herein can have a primary site of tumor in the lower urinary tract and liver metastases. In certain embodiments, the human subjects for whom the methods provided herein can have a primary site of tumor in the lower urinary tract and at least 1 Bellmunt risk factor. In other embodiments, the human subjects for whom the methods provided herein can have a primary site of tumor in the lower urinary tract and ECOG performance status score of 0. In further embodiments, the human subjects for whom the methods provided herein can have visceral metastases and liver metastases. In one embodiment, the human subjects for whom the methods provided herein can have visceral metastases and at least 1 Bellmunt risk factor. In some embodiments, the human subjects for whom the methods provided herein can have visceral metastases and ECOG performance status score of 0. In other embodiments, the human subjects for whom the methods provided herein can have liver metastases and at least 1 Bellmunt risk factor. In yet other embodiments, the human subjects for whom the methods provided herein can have liver metastases and ECOG performance status score of 0. In one embodiment, the human subjects for whom the methods provided herein can have at least 1 Bellmunt risk factor and ECOG performance status score of 0. In other embodiments, the human subjects for whom the methods provided herein can have a primary site of tumor in the lower urinary tract, visceral metastases, and liver metastases. In yet other embodiments, the human subjects for whom the methods provided herein can have a primary site of tumor in the lower urinary tract, visceral metastases and at least 1 Bellmunt risk factor. In further embodiments, the human subjects for whom the methods provided herein can have a primary site of tumor in the lower urinary tract, visceral metastases, and ECOG performance status score of 0. In some embodiments, the human subjects for whom the methods provided herein can have a primary site of tumor in the lower urinary tract, liver metastases and at least 1 Bellmunt risk factor. In certain embodiments, the human subjects for whom the methods provided herein can have a primary site of tumor in the lower urinary tract, liver metastases, and ECOG performance status score of 0. In yet other embodiments, the human subjects for whom the methods provided herein can have a primary site of tumor in the lower urinary tract, at least 1 Bellmunt risk factor and ECOG performance status score of 0. In some embodiments, the human subjects for whom the methods provided herein can have visceral metastases, liver metastases and at least 1 Bellmunt risk factor. In certain embodiments, the human subjects for whom the methods provided herein can have visceral metastases, liver metastases, and ECOG performance status score of 0. In yet other embodiments, the human subjects for whom the methods provided herein can have visceral metastases, at least 1 Bellmunt risk factor and ECOG performance status score of 0. In yet other embodiments, the human subjects for whom the methods provided herein can have liver metastases, at least 1 Bellmunt risk factor and ECOG performance status score of 0. In other embodiments, the human subjects for whom the methods provided herein can have a primary site of tumor in the lower urinary tract, visceral metastases, liver metastases, and at least 1 Bellmunt risk factor. In further embodiments, the human subjects for whom the methods provided herein can have a primary site of tumor in the lower urinary tract, visceral metastases, liver metastases, and ECOG performance status score of 0. In some embodiments, the human subjects for whom the methods provided herein can have a primary site of tumor in the lower urinary tract, visceral metastases, at least 1 Bellmunt risk factor, and ECOG performance status score of 0. In certain embodiments, the human subjects for whom the methods provided herein can have a primary site of tumor in the lower urinary tract, liver metastases, at least 1 Bellmunt risk factor, and ECOG performance status score of 0. In further embodiments, the human subjects for whom the methods provided herein can have visceral metastases, liver metastases, at least 1 Bellmunt risk factor, and ECOG performance status score of 0. In certain embodiments, the human subjects for whom the methods provided herein can have a primary site of tumor in the lower urinary tract, visceral metastases, liver metastases, at least 1 Bellmunt risk factor, and ECOG performance status score of 0. In some embodiments, the human subjects for whom the methods provided herein can have any one of a primary site of tumor in the lower urinary tract, visceral metastases, liver metastases, at least 1 Bellmunt risk factor, and ECOG performance status score of 0. In some embodiments, the human subjects for whom the methods provided herein can have any two of a primary site of tumor in the lower urinary tract, visceral metastases, liver metastases, at least 1 Bellmunt risk factor, and ECOG performance status score of 0, in any combination or permutation. In some embodiments, the human subjects for whom the methods provided herein can have any three of a primary site of tumor in the lower urinary tract, visceral metastases, liver metastases, at least 1 Bellmunt risk factor, and ECOG performance status score of 0, in any combination or permutation. In some embodiments, the human subjects for whom the methods provided herein can have any four of a primary site of tumor in the lower urinary tract, visceral metastases, liver metastases, at least 1 Bellmunt risk factor, and ECOG performance status score of 0, in any combination or permutation. In some embodiments, the human subjects for whom the methods provided herein can have all five of a primary site of tumor in the lower urinary tract, visceral metastases, liver metastases, at least 1 Bellmunt risk factor, and ECOG performance status score of 0.

In further embodiments, the human subjects for whom the methods provided herein can have a primary site of tumor in the upper urinary tract. In one embodiment, the human subjects for whom the methods provided herein can have a primary site of tumor in the upper urinary tract and visceral metastases. In some embodiments, the human subjects for whom the methods provided herein can have a primary site of tumor in the upper urinary tract and liver metastases. In certain embodiments, the human subjects for whom the methods provided herein can have a primary site of tumor in the upper urinary tract and at least 1 Bellmunt risk factor. In other embodiments, the human subjects for whom the methods provided herein can have a primary site of tumor in the upper urinary tract and ECOG performance status score of 0. In other embodiments, the human subjects for whom the methods provided herein can have a primary site of tumor in the upper urinary tract, visceral metastases, and liver metastases. In yet other embodiments, the human subjects for whom the methods provided herein can have a primary site of tumor in the upper urinary tract, visceral metastases and at least 1 Bellmunt risk factor. In further embodiments, the human subjects for whom the methods provided herein can have a primary site of tumor in the upper urinary tract, visceral metastases, and ECOG performance status score of 0. In some embodiments, the human subjects for whom the methods provided herein can have a primary site of tumor in the upper urinary tract, liver metastases and at least 1 Bellmunt risk factor. In certain embodiments, the human subjects for whom the methods provided herein can have a primary site of tumor in the upper urinary tract, liver metastases, and ECOG performance status score of 0. In yet other embodiments, the human subjects for whom the methods provided herein can have a primary site of tumor in the upper urinary tract, at least 1 Bellmunt risk factor and ECOG performance status score of 0. In other embodiments, the human subjects for whom the methods provided herein can have a primary site of tumor in the upper urinary tract, visceral metastases, liver metastases, and at least 1 Bellmunt risk factor. In further embodiments, the human subjects for whom the methods provided herein can have a primary site of tumor in the upper urinary tract, visceral metastases, liver metastases, and ECOG performance status score of 0. In some embodiments, the human subjects for whom the methods provided herein can have a primary site of tumor in the upper urinary tract, visceral metastases, at least 1 Bellmunt risk factor, and ECOG performance status score of 0. In certain embodiments, the human subjects for whom the methods provided herein can have a primary site of tumor in the upper urinary tract, liver metastases, at least 1 Bellmunt risk factor, and ECOG performance status score of 0. In certain embodiments, the human subjects for whom the methods provided herein can have a primary site of tumor in the upper urinary tract, visceral metastases, liver metastases, at least 1 Bellmunt risk factor, and ECOG performance status score of 0. In some embodiments, the human subjects for whom the methods provided herein can have any one of a primary site of tumor in the upper urinary tract, visceral metastases, liver metastases, at least 1 Bellmunt risk factor, and ECOG performance status score of 0. In some embodiments, the human subjects for whom the methods provided herein can have any two of a primary site of tumor in the upper urinary tract, visceral metastases, liver metastases, at least 1 Bellmunt risk factor, and ECOG performance status score of 0, in any combination or permutation. In some embodiments, the human subjects for whom the methods provided herein can have any three of a primary site of tumor in the upper urinary tract, visceral metastases, liver metastases, at least 1 Bellmunt risk factor, and ECOG performance status score of 0, in any combination or permutation. In some embodiments, the human subjects for whom the methods provided herein can have any four of a primary site of tumor in the upper urinary tract, visceral metastases, liver metastases, at least 1 Bellmunt risk factor, and ECOG performance status score of 0, in any combination or permutation. In some embodiments, the human subjects for whom the methods provided herein can have all five of a primary site of tumor in the upper urinary tract, visceral metastases, liver metastases, at least 1 Bellmunt risk factor, and ECOG performance status score of 0.

In further embodiments of the methods provided herein, including the methods of the preceding paragraphs, the human subjects for whom the methods provided herein can be used are human subjects having various other conditions. In one embodiment, the human subjects for whom the methods provided herein can be used also have the conditions of absolute neutrophil count no less than 1.0×10⁹/L. In some embodiments, the human subjects for whom the methods provided herein can be used also have the conditions of platelet count no less than 100′10⁹/L. In certain embodiments, the human subjects for whom the methods provided herein can be used also have the conditions of hemoglobin no less than 9 g/dL. In other embodiments, the human subjects for whom the methods provided herein can be used also have the conditions of serum bilirubin no more than either of 1.5 times of upper limit of normal (ULN) or 3 times ULN for patients with Gilbert's disease. In yet other embodiments, the human subjects for whom the methods provided herein can be used also have the conditions of CrCl no less than 30 mL/min. In another embodiment, the human subjects for whom the methods provided herein can be used also have the conditions of alanine aminotransferase (ALT) and aspartate aminotransferase (AST) no more than 3 fold of ULN. In one embodiment, the human subjects for whom the methods provided herein can be used also have the conditions of absolute neutrophil count no less than 1.0×10⁹/L and platelet count no less than 100×10⁹/L. In some embodiments, the human subjects for whom the methods provided herein can be used also have the conditions of absolute neutrophil count no less than 1.0×10⁹/L and hemoglobin no less than 9 g/dL. In certain embodiments, the human subjects for whom the methods provided herein can be used also have the conditions of absolute neutrophil count no less than 1.0×10⁹/L and serum bilirubin no more than either of 1.5 times of ULN or 3 times ULN for patients with Gilbert's disease. In other embodiments, the human subjects for whom the methods provided herein can be used also have the conditions of absolute neutrophil count no less than 1.0×10⁹/L and CrCl no less than 30 mL/min. In some embodiments, the human subjects for whom the methods provided herein can be used also have the conditions of absolute neutrophil count no less than 1.0×10⁹/L and ALT and AST no more than 3 fold of ULN. In further embodiments, the human subjects for whom the methods provided herein can be used also have the conditions of platelet count no less than 100×10⁹/L and hemoglobin no less than 9 g/dL. In one embodiment, the human subjects for whom the methods provided herein can be used also have the conditions of platelet count no less than 100×10⁹/L and serum bilirubin no more than either of 1.5 times of ULN or 3 times ULN for patients with Gilbert's disease. In some embodiments, the human subjects for whom the methods provided herein can be used also have the conditions of platelet count no less than 100×10⁹/L and CrCl no less than 30 mL/min. In certain embodiments, the human subjects for whom the methods provided herein can be used also have the conditions of platelet count no less than 100×10⁹/L and ALT and AST no more than 3 fold of ULN. In other embodiments, the human subjects for whom the methods provided herein can be used also have the conditions of hemoglobin no less than 9 g/dL and serum bilirubin no more than either of 1.5 times of ULN or 3 times ULN for patients with Gilbert's disease. In yet other embodiments, the human subjects for whom the methods provided herein can be used also have the conditions of hemoglobin no less than 9 g/dL and CrCl no less than 30 mL/min. In some embodiments, the human subjects for whom the methods provided herein can be used also have the conditions of hemoglobin no less than 9 g/dL and ALT and AST no more than 3 fold of ULN. In one embodiment, the human subjects for whom the methods provided herein can be used also have the conditions of serum bilirubin no more than either of 1.5 times of ULN or 3 times ULN for patients with Gilbert's disease and CrCl no less than 30 mL/min. In another embodiment, the human subjects for whom the methods provided herein can be used also have the conditions of serum bilirubin no more than either of 1.5 times of ULN or 3 times ULN for patients with Gilbert's disease and ALT and AST no more than 3 fold of ULN. In another embodiment, the human subjects for whom the methods provided herein can be used also have the conditions of CrCl no less than 30 mL/min and ALT and AST no more than 3 fold of ULN. In other embodiments, the human subjects for whom the methods provided herein can be used also have the conditions of absolute neutrophil count no less than 1.0×10⁹/L, platelet count no less than 100×10⁹/L, and hemoglobin no less than 9 g/dL. In yet other embodiments, the human subjects for whom the methods provided herein can be used also have the conditions of absolute neutrophil count no less than 1.0×10⁹/L, platelet count no less than 100×10⁹/L and serum bilirubin no more than either of 1.5 times of ULN or 3 times ULN for patients with Gilbert's disease. In further embodiments, the human subjects for whom the methods provided herein can be used also have the conditions of absolute neutrophil count no less than 1.0×10⁹/L, platelet count no less than 100×10⁹/L, and CrCl no less than 30 mL/min. In some embodiments, the human subjects for whom the methods provided herein can be used also have the conditions of absolute neutrophil count no less than 1.0×10⁹/L, platelet count no less than 100×10⁹/L. and ALT and AST no more than 3 fold of ULN. In some embodiments, the human subjects for whom the methods provided herein can be used also have the conditions of absolute neutrophil count no less than 1.0×10⁹/L, hemoglobin no less than 9 g/dL and serum bilirubin no more than either of 1.5 times of ULN or 3 times ULN for patients with Gilbert's disease. In certain embodiments, the human subjects for whom the methods provided herein can be used also have the conditions of absolute neutrophil count no less than 1.0-10⁹/L, hemoglobin no less than 9 g/dL, and CrCl no less than 30 mL/min. In some embodiments, the human subjects for whom the methods provided herein can be used also have the conditions of absolute neutrophil count no less than 1.0×10⁹/L, hemoglobin no less than 9 g/dL, and ALT and AST no more than 3 fold of ULN. In yet other embodiments, the human subjects for whom the methods provided herein can be used also have the conditions of absolute neutrophil count no less than 1.0×10⁹/L, serum bilirubin no more than either of 1.5 times of ULN or 3 times ULN for patients with Gilbert's disease and CrCl no less than 30 mL/min. In some embodiments, the human subjects for whom the methods provided herein can be used also have the conditions of absolute neutrophil count no less than 1.0×10⁹/L, serum bilirubin no more than either of 1.5 times of ULN or 3 times ULN for patients with Gilbert's disease and ALT and AST no more than 3 fold of ULN. In some embodiments, the human subjects for whom the methods provided herein can be used also have the conditions of absolute neutrophil count no less than 1.0×10⁹/L, CrCl no less than 30 mL/min and ALT and AST no more than 3 fold of ULN. In some embodiments, the human subjects for whom the methods provided herein can be used also have the conditions of platelet count no less than 100×10⁹/L, hemoglobin no less than 9 g/dL and serum bilirubin no more than either of 1.5 times of ULN or 3 times ULN for patients with Gilbert's disease. In certain embodiments, the human subjects for whom the methods provided herein can be used also have the conditions of platelet count no less than 100×10⁹/L, hemoglobin no less than 9 g/dL, and CrCl no less than 30 mL/min. In some embodiments, the human subjects for whom the methods provided herein can be used also have the conditions of platelet count no less than 100×10⁹/L, hemoglobin no less than 9 g/dL, and ALT and AST no more than 3 fold of ULN. In other embodiments, the human subjects for whom the methods provided herein can be used also have the conditions of platelet count no less than 100×10⁹/L, serum bilirubin no more than either of 1.5 times of ULN or 3 times ULN for patients with Gilbert's disease and CrCl no less than 30 mL/min. In yet other embodiments, the human subjects for whom the methods provided herein can be used also have the conditions of platelet count no less than 100×10⁹/L, serum bilirubin no more than either of 1.5 times of ULN or 3 times ULN for patients with Gilbert's disease and ALT and AST no more than 3 fold of ULN. In some embodiments, the human subjects for whom the methods provided herein can be used also have the conditions of platelet count no less than 100×10⁹/L, CrCl no less than 30 mL/min and ALT and AST no more than 3 fold of ULN. In yet other embodiments, the human subjects for whom the methods provided herein can be used also have the conditions of hemoglobin no less than 9 g/dL, serum bilirubin no more than either of 1.5 times of ULN or 3 times ULN for patients with Gilbert's disease and CrCl no less than 30 mL/min. In other embodiments, the human subjects for whom the methods provided herein can be used also have the conditions of hemoglobin no less than 9 g/dL, serum bilirubin no more than either of 1.5 times of ULN or 3 times ULN for patients with Gilbert's disease and ALT and AST no more than 3 fold of ULN. In certain embodiments, the human subjects for whom the methods provided herein can be used also have the conditions of hemoglobin no less than 9 g/dL, CrCl no less than 30 mL/min and ALT and AST no more than 3 fold of ULN. In some embodiments, the human subjects for whom the methods provided herein can be used also have the conditions of serum bilirubin no more than either of 1.5 times of ULN or 3 times ULN for patients with Gilbert's disease, CrCl no less than 30 mL/min and ALT and AST no more than 3 fold of ULN. In other embodiments, the human subjects for whom the methods provided herein can be used also have the conditions of absolute neutrophil count no less than 1.0×10⁹/L, platelet count no less than 100×10⁹/L, hemoglobin no less than 9 g/dL, and serum bilirubin no more than either of 1.5 times of ULN or 3 times ULN for patients with Gilbert's disease. In further embodiments, the human subjects for whom the methods provided herein can be used also have the conditions of absolute neutrophil count no less than 1.0×10⁹/L, platelet count no less than 100×10⁹/L, hemoglobin no less than 9 g/dL, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, serum bilirubin no more than either of 1.5 times of ULN or 3 times ULN for patients with Gilbert's disease, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, serum bilirubin no more than either of 1.5 times of ULN or 3 times ULN for patients with Gilbert's disease, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, hemoglobin no less than 9 g/dL, serum bilirubin no more than either of 1.5 times of ULN or 3 times ULN for patients with Gilbert's disease, and CrCl no less than 30 ml/min. In certain embodiments, the human subjects for whom the methods provided herein can be used also have the conditions of absolute neutrophil count no less than 1.0×10⁹/L, hemoglobin no less than 9 g/dL, serum bilirubin no more than either of 1.5 times of ULN or 3 times ULN for patients with Gilbert's disease, and ALT and AST no more than 3 fold of ULN. In certain embodiments, the human subjects for whom the methods provided herein can be used also have the conditions of absolute neutrophil count no less than 1.0×10⁹/L, hemoglobin no less than 9 g/dL, CrCl no less than 30 mL/min, and ALT and AST no more than 3 fold of ULN. In yet other embodiments, the human subjects for whom the methods provided herein can be used also have the conditions of absolute neutrophil count no less than 1.0×10⁹/L, serum bilirubin no more than either of 1.5 times of ULN or 3 times ULN for patients with Gilbert's disease, CrCl no less than 30 mL/min, and ALT and AST no more than 3 fold of ULN. In further embodiments, the human subjects for whom the methods provided herein can be used also have the conditions of platelet count no less than 100×10⁹/L, hemoglobin no less than 9 g/dL, serum bilirubin no more than either of 1.5 times of ULN or 3 times ULN for patients with Gilbert's disease, and CrCl no less than 30 mL/min. In some embodiments, the human subjects for whom the methods provided herein can be used also have the conditions of platelet count no less than 100×10⁹/L, hemoglobin no less than 9 g/dL, serum bilirubin no more than either of 1.5 times of ULN or 3 times ULN for patients with Gilbert's disease, and ALT and AST no more than 3 fold of ULN. In further embodiments, the human subjects for whom the methods provided herein can be used also have the conditions of platelet count no less than 100×10⁹/L, hemoglobin no less than 9 g/dL, CrCl no less than 30 mL/min, and ALT and AST no more than 3 fold of ULN. In some embodiments, the human subjects for whom the methods provided herein can be used also have the conditions of platelet count no less than 100×10⁹/L, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, platelet count no less than 100×10⁹/L, serum bilirubin no more than either of 1.5 times of ULN or 3 times ULN for patients with Gilbert's disease, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of platelet count no less than 10×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease. CrCl no less than 30 mL/min, and ALT and AST no more than 3 fold of ULN. In some embodiments, the human subjects for whom the methods provided herein can be used also have the conditions of any one of absolute neutrophil count no less than 1.0×10⁹/L, platelet count no less than 100′10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of any two of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease. CrCl no less than 30 mL/min, and ALT and AST no more than 3 fold of ULN, in any combination or permutation. In some embodiments, the human subjects for whom the methods provided herein can be used also have the conditions of any three of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of any four of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of any five of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of all six of absolute neutrophil count no less than 1.0×10⁹′L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN.

In other embodiments of the methods provided herein, including the methods of the preceding paragraphs, the human subjects for whom the methods provided herein can be used are human subjects free from certain conditions. In one embodiment, the human subjects for whom the methods provided herein can have no more than Grade 2 sensory or motor neuropathy. In some embodiments, the human subjects for whom the methods provided herein can have no active central nervous system metastases. In certain embodiments, the human subjects for whom the methods provided herein can have no uncontrolled diabetes. In one embodiment, the human subjects for whom the methods provided herein can have no more than Grade 2 sensory or motor neuropathy and no active central nervous system metastases. In some embodiments, the human subjects for whom the methods provided herein can have no more than Grade 2 sensory or motor neuropathy and no uncontrolled diabetes. In further embodiments, the human subjects for whom the methods provided herein can have no active central nervous system metastases and no uncontrolled diabetes. In yet other embodiments, the human subjects for whom the methods provided herein can have no more than Grade 2 sensory or motor neuropathy, no active central nervous system metastases, and no uncontrolled diabetes. In some embodiments, the human subjects for whom the methods provided herein can have any one of no more than Grade 2 sensory or motor neuropathy, no active central nervous system metastases, and no uncontrolled diabetes. In some embodiments, the human subjects for whom the methods provided herein can have any two of no more than Grade 2 sensory or motor neuropathy, no active central nervous system metastases, and no uncontrolled diabetes, in any combination or permutation. In some embodiments, the human subjects for whom the methods provided herein can have all three of no more than Grade 2 sensory or motor neuropathy, no active central nervous system metastases, and no uncontrolled diabetes. In one embodiment of the methods provided in this paragraph, the uncontrolled diabetes is determined by hemoglobin A1c (HbA1c) no less than 8%. In some embodiments of the methods provided in this paragraph, the uncontrolled diabetes is determined by HbA1c between 7 and 8% with associated diabetes symptoms that are not otherwise explained. In further embodiments of the methods provided in this paragraph, the associated diabetes symptoms comprise or consist of polyuria. In some other embodiments of the methods provided in this paragraph, the associated diabetes symptoms comprise or consist of polydipsia. In yet other embodiments of the methods provided in this paragraph, the associated diabetes symptoms comprise or consist of both polyuria and polydipsia.

In some embodiments of the methods provided herein, the CrCl is measured by 24 hour urine collection. In other embodiments of the methods provided herein, the CrCl is estimated by the Cockcroft-Gault criteria.

In some embodiments of the methods provided herein, the subject has been treated with one or more other cancer treatments. In certain embodiments of the methods provided herein, the urothelial cancer, including locally advanced or metastatic urothelial cancer, has been treated with one or more other cancer treatments.

In some embodiments, the CPI provided for the methods can comprise or consist of any CPI as described in this Section (Section 5.2.1).

In all the methods provided herein and specifically those described in the preceding paragraphs: the ADCs that can be used are described in Sections 3, 5.2, 5.3, 5.4, 5.5, and 6, selection of patients for treatment is described herein and exemplified in this Section (Section 5.2) and Sections 3 and 6, dosing regimens and pharmaceutical composition for administering the therapeutic agent are described in this Section (Section 5.2), Sections 5.4, 5.6, 5.7 and 6 below, the biomarkers that can be used for identifying the therapeutic agents, selecting the patients, determining the outcome of these methods, and/or serving as criteria in any way for these methods are described herein and exemplified in this Section (Section 5.2, including 5.2.1 and 5.2.2) and Section 6, the biomarkers can be determined as described in Section 5.8 or as known in the art, therapeutic outcomes for the methods provided herein are described in this Section (Section 5.2 including Section 5.2.1.4) and Sections 3 and 6, additional therapeutic outcomes for the methods provided herein can be improvement of the biomarkers described herein, for example, those described and exemplified in this Section (Section 5.2 including 5.2.2) and Sections 3 and 6, and combination therapies including the ADCs and other therapeutic agents are described in this Section (Section 5.2) and in Section 5.5. Therefore, a person skilled in the art would understand that the methods provided herein include all permutations and combinations of the patients, therapeutic agents, dosing regiments, biomarkers, and therapeutic outcomes as described above and below.

In certain embodiments, the methods provided herein are used for treating subjects having urothelial cancers that express 191P4D12 RNA, express 191P4D12 protein, or express both 191P4D12 RNA and 191P4D12 protein. In one embodiment, the methods provided herein are used for treating subjects who have urothelial cancers that express 191P4D12 RNA, express 191P4D12 protein, or express both 191P4D12 RNA and 191P4D12 protein and who have been previously treated with a CPI.

In certain embodiments, the methods provided herein are used for treating subjects having locally advanced urothelial cancers that express 191P4D12 RNA, express 191P4D12 protein, or express both 191P4D12 RNA and 191P4D12 protein. In one embodiment, the methods provided herein are used for treating subjects who have locally advanced urothelial cancers that express 191P4D12 RNA, express 191P4D12 protein, or express both 191P4D12 RNA and 191P4D12 protein and who have been previously treated with a CPI.

In certain embodiments, the methods provided herein are used for treating subjects having metastatic cancers that express 191P4D12 RNA, express 191P4D12 protein, or express both 191P4D12 RNA and 191P4D12 protein. In one embodiment, the methods provided herein are used for treating subjects who have metastatic urothelial cancers that express 191P4D12 RNA, express 191P4D12 protein, or express both 191P4D12 RNA and 191P4D12 protein and who have been previously treated with a CPI.

In some embodiments, the 191P4D12 RNA expression in the cancers is determined by polynucleotide hybridization, sequencing (assessing the relative abundance of the sequences), and/or PCR (including RT-PCR). In some embodiments, the 191P4D12 protein expression in the cancers is determined by IHC, analysis in fluorescence-activated cell sorting (FACS), and/or western blotting. In some embodiments, the 191P4D12 protein expression in the cancers is determined by more than one method. In some embodiments, the 191P4D12 protein expression in the cancers is determined by two methods of IHC.

In some embodiments, the locally advanced or metastatic urothelial cancers are confirmed histologically, cytologically, or both histologically and cytologically. In some embodiments, the locally advanced or metastatic bladder cancers are confirmed histologically, cytologically, or both histologically and cytologically.

5.2.1.3 Prior Treatments Including Prior CPI Treatments

In some embodiments, the subjects that can be treated in the methods provided herein include subjects who received one or more other treatments for cancer. In some embodiments, the subjects that can be treated in the methods provided herein include subjects who received one or more other treatments for cancer and whose cancer progressed or relapsed following the one or more treatments. Such one or more treatments include, for example, one or more lines of immune checkpoint inhibitor therapies, chemotherapies, and both immune checkpoint inhibitor therapies and chemotherapies. In some embodiments, the subjects that can be treated in the methods provided herein include subjects whose cancers progressed or relapsed following a therapy with a CPI.

In some specific embodiments, the subjects that can be treated in the methods provided herein include subjects whose cancers progressed or relapsed following a therapy with a CPI. In some specific embodiments, the subjects that can be treated in the methods provided herein include subjects whose cancer progressed or relapsed following a therapy with a CPI in the neoadjuvant setting. In some specific embodiments, the subjects that can be treated in the methods provided herein include subjects whose cancer progressed or relapsed following a therapy with a CPI in the adjuvant setting. In some specific embodiments, the subjects that can be treated in the methods provided herein include subjects whose cancer progressed or relapsed following a therapy with a CPI in the locally advanced setting. In some specific embodiments, the subjects that can be treated in the methods provided herein include subjects whose cancer progressed or relapsed following a therapy with a CPI in the metastatic setting. In some specific embodiments, the subjects that can be treated in the methods provided herein include subjects whose cancer progressed or relapsed following a therapy with a CPI in the neoadjuvant, locally advanced setting. In some specific embodiments, the subjects that can be treated in the methods provided herein include subjects whose cancer progressed or relapsed following a therapy with a CPI in the neoadjuvant, metastatic setting. In some specific embodiments, the subjects that can be treated in the methods provided herein include subjects whose cancer progressed or relapsed following a therapy with a CPI in the adjuvant, locally advanced setting. In some specific embodiments, the subjects that can be treated in the methods provided herein include subjects whose cancer progressed or relapsed following a therapy with a CPI in the adjuvant, metastatic setting.

In some specific embodiments, the subjects that can be treated in the methods provided herein include subjects whose cancers progressed or relapsed following a therapy with a PD-1 inhibitor. In some specific embodiments, the subjects that can be treated in the methods provided herein include subjects whose cancer progressed or relapsed following a therapy with a PD-1 inhibitor in the neoadjuvant setting. In some specific embodiments, the subjects that can be treated in the methods provided herein include subjects whose cancer progressed or relapsed following a therapy with a PD-1 inhibitor in the adjuvant setting. In some specific embodiments, the subjects that can be treated in the methods provided herein include subjects whose cancer progressed or relapsed following a therapy with a PD-1 inhibitor in the locally advanced setting. In some specific embodiments, the subjects that can be treated in the methods provided herein include subjects whose cancer progressed or relapsed following a therapy with a PD-1 inhibitor in the metastatic setting. In some specific embodiments, the subjects that can be treated in the methods provided herein include subjects whose cancer progressed or relapsed following a therapy with a PD-1 inhibitor in the neoadjuvant, locally advanced setting. In some specific embodiments, the subjects that can be treated in the methods provided herein include subjects whose cancer progressed or relapsed following a therapy with a PD-1 inhibitor in the neoadjuvant, metastatic setting. In some specific embodiments, the subjects that can be treated in the methods provided herein include subjects whose cancer progressed or relapsed following a therapy with a PD-1 inhibitor in the adjuvant, locally advanced setting. In some specific embodiments, the subjects that can be treated in the methods provided herein include subjects whose cancer progressed or relapsed following a therapy with a PD-1 inhibitor in the adjuvant, metastatic setting.

In some specific embodiments, the subjects that can be treated in the methods provided herein include subjects whose cancers progressed or relapsed following a therapy with a PD-L1 inhibitor. In some specific embodiments, the subjects that can be treated in the methods provided herein include subjects whose cancer progressed or relapsed following a therapy with a PD-L1 inhibitor in the neoadjuvant setting. In some specific embodiments, the subjects that can be treated in the methods provided herein include subjects whose cancer progressed or relapsed following a therapy with a PD-L1 inhibitor in the adjuvant setting. In some specific embodiments, the subjects that can be treated in the methods provided herein include subjects whose cancer progressed or relapsed following a therapy with a PD-L1 inhibitor in the locally advanced setting. In some specific embodiments, the subjects that can be treated in the methods provided herein include subjects whose cancer progressed or relapsed following a therapy with a PD-L1 inhibitor in the metastatic setting. In some specific embodiments, the subjects that can be treated in the methods provided herein include subjects whose cancer progressed or relapsed following a therapy with a PD-L1 inhibitor in the neoadjuvant, locally advanced setting. In some specific embodiments, the subjects that can be treated in the methods provided herein include subjects whose cancer progressed or relapsed following a therapy with a PD-L1 inhibitor in the neoadjuvant, metastatic setting. In some specific embodiments, the subjects that can be treated in the methods provided herein include subjects whose cancer progressed or relapsed following a therapy with a PD-L1 inhibitor in the adjuvant, locally advanced setting. In some specific embodiments, the subjects that can be treated in the methods provided herein include subjects whose cancer progressed or relapsed following a therapy with a PD-L1 inhibitor in the adjuvant, metastatic setting.

In certain embodiments, the subjects that can be treated in the methods provided herein include those whose cancers have progressed or relapsed other treatments for cancers within 1, 2, 3, 4, 5, 6, 7, 8, 9, 10, 11, 12, 13, 14, 15, 16, 17, 18, 19, 20, 21, 22, 23, or 24 months after the other treatments, including for example and not by way of limitation, any or any combination of the treatments described in the preceding paragraphs. In some particular embodiment, the cancers in the subjects have progressed or relapsed within 6 months after the other cancer therapies, including CPI therapies such as PD-1 inhibitor or PD-L1 inhibitor therapies. In further embodiments, the cancers in the subjects have progressed or relapsed within 12 months after the other cancer therapies, including CPI therapies such as PD-1 inhibitor or PD-L1 inhibitor therapies.

In some embodiments, the subjects that can be treated in the methods provided herein have certain phenotypic or genotypic characteristics. In some embodiments, the subjects have any permutation and combination of the phenotypic or genotypic characteristics described herein.

In some embodiments, the phenotypic or genotypic characteristics are determined histologically, cytologically, or both histologically and cytologically. In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference. In some embodiments, the phenotypic or genotypic characteristics are determined by sequencing including the next generation sequencing (e.g. NGS from Illumina, Inc), DNA hybridization, and/or RNA hybridization.

In various aspects or embodiments of the methods provided herein, including the methods provided in this Section (Section 5.2) such as the methods provided in this and the preceding paragraphs, the methods involve a prior treatment with an immune checkpoint inhibitor as provided in the method. As used herein, the term “immune checkpoint inhibitor” or “checkpoint inhibitor” refers to molecules that totally or partially reduce, inhibit, interfere with or modulate one or more checkpoint proteins. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). Other exemplary checkpoint proteins include LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In certain embodiments, the checkpoint inhibitor for the methods provided herein can be an inhibitors or activators against a checkpoint protein that upregulated in cancer. In some specific embodiments, the checkpoint inhibitor for the methods provided herein can be an inhibitors or activators against a checkpoint protein including LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. In some embodiments, the checkpoint inhibitor for the methods provided herein can be an inhibitors or activators selected from the group consisting of a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, or a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or a therapeutic agent targeting HLA. Such inhibitors, activators, or therapeutic agents are further provided below.

In some embodiments, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In certain embodiments, the checkpoint inhibitor is a PD-1/PD-L1 inhibitor. Examples of PD-1/PD-L1 inhibitors include, but are not limited to, those described in U.S. Pat. Nos. 7,488,802; 7,943,743, 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475. SCH 900475, or lambrolizumab). In one embodiment, the anti-PD-1 antibody is nivolumab. Nivolumab is a human IgG4 anti-PD-1 monoclonal antibody, and is marketed under the trade name Opdivor™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the PD-1 antibody is camrelizumab. In a further embodiment, the PD-1 antibody is sintilimab. In some embodiments, the PD-1 antibody is tislelizumab. In certain embodiments, the PD-1 antibody is TSR-042. In yet another embodiment, the PD-1 antibody is PDR001. In yet another embodiment, the PD-1 antibody is toripalimab.

In certain embodiments, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®). In a further embodiment, the PD-L1 inhibitor is avelumab.

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is a VTCN inhibitor. In one embodiment, the VTCN inhibitor is FPA150.

In one embodiment, the checkpoint inhibitor is an IDO inhibitor. In one embodiment, the IDO inhibitor is INCB024360. In another embodiment, the IDO inhibitor is indoximod. In one embodiment, the IDO inhibitor is epacadostat. In another embodiment, the IDO inhibitor is BMS986205. In yet another embodiment, the IDO inhibitor is Navoximod. In one embodiment, the IDO inhibitor is PF-06840003. In another embodiment, the IDO inhibitor is KHK2455. In yet another embodiment, the IDO inhibitor is RG70099. In one embodiment, the IDO inhibitor is IOM-E. In another embodiment, the IDO inhibitor is or IOM-D.

In some embodiments, the checkpoint inhibitor is a TIGIT inhibitor. In certain embodiments, the TIGIT inhibitor is an anti-TIGIT antibody. In one embodiment, the TIGIT inhibitor is MTIG7192A. In another embodiment, the TIGIT inhibitor is BMS-986207. In yet another embodiment, the TIGIT inhibitor is OMP-313M32. In one embodiment, the TIGIT inhibitor is MK-7684. In another embodiment, the TIGIT inhibitor is AB154. In yet another embodiment, the TIGIT inhibitor is CGEN-15137. In one embodiment, the TIGIT inhibitor is SEA-TIGIT. In another embodiment, the TIGIT inhibitor is ASP8374. In yet another embodiment, the TIGIT inhibitor is AJUD008.

In some embodiments, the checkpoint inhibitor is a VSIR inhibitor. In certain embodiments, the VSIR inhibitor is an anti-VSIR antibody. In one embodiment, the VSIR inhibitor is MTIG7192A. In another embodiment, the VSIR inhibitor is CA-170. In yet another embodiment, the VSIR inhibitor is JNJ 61610588. In one embodiment, the VSIR inhibitor is HMBD-002.

In some embodiments, the checkpoint inhibitor is a TIM3 inhibitor. In certain embodiments, the TIM3 inhibitor is an anti-TIM3 antibody. In one embodiment, the TIM3 inhibitor is AJUD009.

In some embodiments, the checkpoint inhibitor is a CD25 (IL2RA) inhibitor. In certain embodiments, the CD25 (IL2RA) inhibitor is an anti-CD25 (IL2RA) antibody. In one embodiment, the CD25 (IL2RA) inhibitor is daclizumab. In another embodiment, the CD25 (IL2RA) inhibitor is basiliximab.

In some embodiments, the checkpoint inhibitor is an IFNAR1 inhibitor. In certain embodiments, the IFNAR1 inhibitor is an anti-IFNAR1 antibody. In one embodiment, the IFNAR1 inhibitor is anifrolumab. In another embodiment, the IFNAR1 inhibitor is sifalimumab.

In some embodiments, the checkpoint inhibitor is a CSF1R inhibitor. In certain embodiments, the CSF1R inhibitor is an anti-CSF1R antibody. In one embodiment, the CSF1R inhibitor is pexidartinib. In another embodiment, the CSF1R inhibitor is emactuzumab. In yet another embodiment, the CSF1R inhibitor is cabiralizumab. In one embodiment, the CSF1R inhibitor is ARRY-382. In another embodiment, the CSF1R inhibitor is BLZ945. In yet another embodiment, the CSF1R inhibitor is AJUD010. In one embodiment, the CSF1R inhibitor is AMG820. In another embodiment, the CSF1R inhibitor is IMC-CS4. In yet another embodiment, the CSF1R inhibitor is JNJ-40346527. In one embodiment, the CSF1R inhibitor is PLX5622. In another embodiment, the CSF1R inhibitor is FPA008.

In some embodiments, the checkpoint inhibitor is a therapeutic agent targeting HLA. In certain embodiments, the therapeutic agent targeting HLA is an anti-HLA antibody. In one embodiment, the therapeutic agent targeting HLA is GSK01. In another embodiment, the therapeutic agent targeting HLA is IMC-C103C. In yet another embodiment, the therapeutic agent targeting HLA is IMC-F106C. In one embodiment, the therapeutic agent targeting HLA is IMC-G107C. In another embodiment, the therapeutic agent targeting HLA is ABBV-184.

In certain embodiments, the immune checkpoint inhibitors provided herein include two or more of the checkpoint inhibitors described herein (including checkpoint inhibitors of the same or different class). Moreover, the methods described herein can be used in combination with one or more second active agents as described herein where appropriate for treating diseases described herein and understood in the art.

In some embodiments, the checkpoint inhibitor is administered after the administration of the ADCs provided herein. In other embodiments, the checkpoint inhibitor is administered simultaneously (e.g., in the same dosing period) with the ADCs provided herein. In yet other embodiments, the checkpoint inhibitor is administered after the administration of the ADCs provided herein.

In some embodiments, the amount of the checkpoint inhibitor for the various methods provided herein can be determined by standard clinical techniques. In certain embodiments, the amount of the checkpoint inhibitor for the various methods are provided in Section 5.6.

In some embodiments, the subjects that can be treated in the methods provided herein is a mammal. In some embodiments, the subjects that can be treated in the methods provided herein is a human.

5.2.1.4 Therapeutic Outcome of the Methods Provided Herein

Despite the poor prognosis for cisplatin ineligible human subjects who as described above are frail, suffer from multiple comorbidities beyond their urothelial cancer/bladder cancer and are not able to tolerate additional treatment beyond immunotherapy, the methods provided herein, including in methods described in this Section (Section 5.2) and Sections 3 and 6, can provide beneficial therapeutic outcomes for these cisplatin ineligible human subjects. In one embodiment, the human subject has a complete response following the treatment by a method provided herein. In another embodiment, the human subject has a partial response following the treatment by a method provided herein.

In some embodiments, the response (complete or partial response) is determined by evaluating the tumor or cancer site (lesions). The criteria for determining complete response (CR), partial response (PR), progressive disease (PD), and stable disease (SD) are described in Table 28.

The therapeutic outcome of the methods provided herein thus can be evaluated based on any one or more of the response criteria described above. In one embodiment, the human subject has a partial response following the treatment by a method provided herein, wherein the partial response is defined by an at least or about 30% decrease in the sum of the diameters of target lesions, taking as reference the baseline sum diameters. In another embodiment, the human subject has a partial response following the treatment by a method provided herein, wherein the partial response is defined by an at least or about 35% decrease in the sum of the diameters of target lesions, taking as reference the baseline sum diameters. In a further embodiment, the human subject has a partial response following the treatment by a method provided herein, wherein the partial response is defined by an at least or about 40% decrease in the sum of the diameters of target lesions, taking as reference the baseline sum diameters. In yet another embodiment, the human subject has a partial response following the treatment by a method provided herein, wherein the partial response is defined by an at least or about 45% decrease in the sum of the diameters of target lesions, taking as reference the baseline sum diameters. In one embodiment, the human subject has a partial response following the treatment by a method provided herein, wherein the partial response is defined by an at least or about 50% decrease in the sum of the diameters of target lesions, taking as reference the baseline sum diameters. In another embodiment, the human subject has a partial response following the treatment by a method provided herein, wherein the partial response is defined by an at least or about 55% decrease in the sum of the diameters of target lesions, taking as reference the baseline sum diameters. In a further embodiment, the human subject has a partial response following the treatment by a method provided herein, wherein the partial response is defined by an at least or about 60% decrease in the sum of the diameters of target lesions, taking as reference the baseline sum diameters. In yet another embodiment, the human subject has a partial response following the treatment by a method provided herein, wherein the partial response is defined by an at least or about 65% decrease in the sum of the diameters of target lesions, taking as reference the baseline sum diameters. In one embodiment, the human subject has a partial response following the treatment by a method provided herein, wherein the partial response is defined by an at least or about 70% decrease in the sum of the diameters of target lesions, taking as reference the baseline sum diameters. In another embodiment, the human subject has a partial response following the treatment by a method provided herein, wherein the partial response is defined by an at least or about 75% decrease in the sum of the diameters of target lesions, taking as reference the baseline sum diameters. In a further embodiment, the human subject has a partial response following the treatment by a method provided herein, wherein the partial response is defined by an at least or about 80% decrease in the sum of the diameters of target lesions, taking as reference the baseline sum diameters. In yet another embodiment, the human subject has a partial response following the treatment by a method provided herein, wherein the partial response is defined by an at least or about 85% decrease in the sum of the diameters of target lesions, taking as reference the baseline sum diameters. In one embodiment, the human subject has a partial response following the treatment by a method provided herein, wherein the partial response is defined by an at least or about 90% decrease in the sum of the diameters of target lesions, taking as reference the baseline sum diameters. In another embodiment, the human subject has a partial response following the treatment by a method provided herein, wherein the partial response is defined by an at least or about 95% decrease in the sum of the diameters of target lesions, taking as reference the baseline sum diameters. In some embodiments, the diameter is determined according to the longest diameter of a lesion. In certain embodiments, the diameter is determined according to the longest diameter of a lesion in the plane of measurement. In some embodiments, the diameter is determined according to the longest diameter of a lesion in the plane of measurement with a minimal size of 10 mm by CT scan. In certain embodiments, the diameter is determined according to the longest diameter of a lesion in the plane of measurement with a minimal size of 10 mm by CT scan and CT slice thickness no greater than 5 mm.

The therapeutic outcomes of the methods provided herein can also be evaluated based on whether the disease is stable following the treatment. In one embodiment, the human subject has a stable disease following the treatment by a method provided herein. In another embodiment, the human subject does not have a progressive disease following the treatment by a method provided herein.

Alternatively, therapeutic outcomes based on the complete response, partial response, or stable disease can be evaluated with respect to a population of human subjects treated by a method provided herein by evaluating the percentage of the subjects having complete response, partial response, or stable disease in the treated population. As such, in some embodiments, the therapeutic outcome or efficacy measure applies to outcomes achieved by actually treating a population of subjects. In other embodiments, the therapeutic outcome or efficacy measure refers to the outcome or efficacy that is capable of being achieved if a population of human subjects was treated with a method as disclosed herein. While the following sections discuss the treatment of an actual population of human subjects, is should be understood that corresponding methods in which the outcome or efficacy measure is capable of being achieved in a patient population are also encompassed herein. In short, both scenarios described above apply to the following sections; only one scenario is described below in the interest of simplicity and to avoid redundancy.

In some embodiments of the methods provided herein, including in Sections 3, 5.3, and 6 and this Section (Section 5.2), the ADC is enfortumab vedotin. In certain embodiments of the methods provided herein, including in Sections 3, 5.3, and 6 and this Section (Section 5.2), the ADC is a biosimilar of enfortumab vedotin.

In one embodiment, a population of the human subjects is treated by a method provided herein, wherein the percentage of the subjects having complete response in the treated population is at least or about 10%. In another embodiment, a population of the human subjects is treated by a method provided herein, wherein the percentage of the subjects having complete response in the treated population is at least or about 15%. In a further embodiment, a population of the human subjects is treated by a method provided herein, wherein the percentage of the subjects having complete response in the treated population is at least or about 20%. In yet another embodiment, a population of the human subjects is treated by a method provided herein, wherein the percentage of the subjects having complete response in the treated population is at least or about 20.2%. In one embodiment, a population of the human subjects is treated by a method provided herein, wherein the percentage of the subjects having complete response in the treated population is at least or about 22%. In yet another embodiment, a population of the human subjects is treated by a method provided herein, wherein the percentage of the subjects having complete response in the treated population is at least or about 22.5%. In another embodiment, a population of the human subjects is treated by a method provided herein, wherein the percentage of the subjects having complete response in the treated population is at least or about 23%. In one embodiment, a population of the human subjects is treated by a method provided herein, wherein the percentage of the subjects having complete response in the treated population is at least or about 25%. In another embodiment, a population of the human subjects is treated by a method provided herein, wherein the percentage of the subjects having complete response in the treated population is at least or about 30%. In a further embodiment, a population of the human subjects is treated by a method provided herein, wherein the percentage of the subjects having complete response in the treated population is at least or about 35%. In yet another embodiment, a population of the human subjects is treated by a method provided herein, wherein the percentage of the subjects having complete response in the treated population is at least or about 40%.

Similarly, using percentage of partial response as the criteria, in one embodiment, a population of the human subjects is treated by a method provided herein, wherein the percentage of the subjects having partial response in the treated population is at least or about 10%. In another embodiment, a population of the human subjects is treated by a method provided herein, wherein the percentage of the subjects having partial response in the treated population is at least or about 15%. In a further embodiment, a population of the human subjects is treated by a method provided herein, wherein the percentage of the subjects having partial response in the treated population is at least or about 20%. In yet another embodiment, a population of the human subjects is treated by a method provided herein, wherein the percentage of the subjects having partial response in the treated population is at least or about 25%. In one embodiment, a population of the human subjects is treated by a method provided herein, wherein the percentage of the subjects having partial response in the treated population is at least or about 28%. In one embodiment, a population of the human subjects is treated by a method provided herein, wherein the percentage of the subjects having partial response in the treated population is at least or about 28.1%. In one embodiment, a population of the human subjects is treated by a method provided herein, wherein the percentage of the subjects having partial response in the treated population is at least or about 29%. In another embodiment, a population of the human subjects is treated by a method provided herein, wherein the percentage of the subjects having partial response in the treated population is at least or about 30%. In another embodiment, a population of the human subjects is treated by a method provided herein, wherein the percentage of the subjects having partial response in the treated population is at least or about 31%. In another embodiment, a population of the human subjects is treated by a method provided herein, wherein the percentage of the subjects having partial response in the treated population is at least or about 31.5%. In a further embodiment, a population of the human subjects is treated by a method provided herein, wherein the percentage of the subjects having partial response in the treated population is at least or about 35%. In yet another embodiment, a population of the human subjects is treated by a method provided herein, wherein the percentage of the subjects having partial response in the treated population is at least or about 40%. In one embodiment, a population of the human subjects is treated by a method provided herein, wherein the percentage of the subjects having partial response in the treated population is at least or about 45%. In another embodiment, a population of the human subjects is treated by a method provided herein, wherein the percentage of the subjects having partial response in the treated population is at least or about 50%.

Likewise, objective response rate, which is the sum of percentage of subjects having completed response and those having partial response, can be used as the evaluation criteria for the therapeutic outcome in the human subjects treated by a method provided herein. In one embodiment, a population of the human subjects is treated by a method provided herein, wherein objective response rate in the treated population is at least or about 20%. In one embodiment, a population of the human subjects is treated by a method provided herein, wherein objective response rate in the treated population is at least or about 25%. In another embodiment, a population of the human subjects is treated by a method provided herein, wherein objective response rate in the treated population is at least or about 30%. In a further embodiment, a population of the human subjects is treated by a method provided herein, wherein objective response rate in the treated population is at least or about 35%. In yet another embodiment, a population of the human subjects is treated by a method provided herein, wherein objective response rate in the treated population is at least or about 40%. In yet another embodiment, a population of the human subjects is treated by a method provided herein, wherein objective response rate in the treated population is at least or about 40.8%. In one embodiment, a population of the human subjects is treated by a method provided herein, wherein objective response rate in the treated population is at least or about 45%. In another embodiment, a population of the human subjects is treated by a method provided herein, wherein objective response rate in the treated population is at least or about 50%. In one embodiment, a population of the human subjects is treated by a method provided herein, wherein objective response rate in the treated population is at least or about 50.6%. In a further embodiment, a population of the human subjects is treated by a method provided herein, wherein objective response rate in the treated population is at least or about 51%. In yet another embodiment, a population of the human subjects is treated by a method provided herein, wherein objective response rate in the treated population is at least or about 51.7%. In one embodiment, a population of the human subjects is treated by a method provided herein, wherein objective response rate in the treated population is at least or about 55%. In another embodiment, a population of the human subjects is treated by a method provided herein, wherein objective response rate in the treated population is at least or about 60%. In a further embodiment, a population of the human subjects is treated by a method provided herein, wherein objective response rate in the treated population is at least or about 65%. In yet another embodiment, a population of the human subjects is treated by a method provided herein, wherein objective response rate in the treated population is at least or about 70%. In one embodiment, a population of the human subjects is treated by a method provided herein, wherein objective response rate in the treated population is at least or about 75%. In another embodiment, a population of the human subjects is treated by a method provided herein, wherein objective response rate in the treated population is at least or about 80%. In a further embodiment, a population of the human subjects is treated by a method provided herein, wherein objective response rate in the treated population is at least or about 85%. In yet another embodiment, a population of the human subjects is treated by a method provided herein, wherein objective response rate in the treated population is at least or about 90%.

In one embodiment, a population of the human subjects is treated by a method provided herein, wherein objective response rate in the treated population ranges from 40% to 63%. In one embodiment, a population of the human subjects is treated by a method provided herein, wherein objective response rate in the treated population ranges from 40% to 63%. In another embodiment, a population of the human subjects is treated by a method provided herein, wherein objective response rate in the treated population ranges from 40% to 60%. In a further embodiment, a population of the human subjects is treated by a method provided herein, wherein objective response rate in the treated population ranges from 40% to 55%. In yet another embodiment, a population of the human subjects is treated by a method provided herein, wherein objective response rate in the treated population ranges from 40% to 50%. In one embodiment, a population of the human subjects is treated by a method provided herein, wherein objective response rate in the treated population ranges from 40% to 45%. In another embodiment, a population of the human subjects is treated by a method provided herein, wherein objective response rate in the treated population ranges from 45% to 63%. In a further embodiment, a population of the human subjects is treated by a method provided herein, wherein objective response rate in the treated population ranges from 50% to 63%. In yet another embodiment, a population of the human subjects is treated by a method provided herein, wherein objective response rate in the treated population ranges from 55% to 63%. In one embodiment, a population of the human subjects is treated by a method provided herein, wherein objective response rate in the treated population ranges from 60% to 63%. In another embodiment, a population of the human subjects is treated by a method provided herein, wherein objective response rate in the treated population ranges from 40% to 62%. In one embodiment, a population of the human subjects is treated by a method provided herein, wherein objective response rate in the treated population ranges from 40% to 61%. In another embodiment, a population of the human subjects is treated by a method provided herein, wherein objective response rate in the treated population ranges from 39.8% to 61.3%. In another embodiment, a population of the human subjects is treated by a method provided herein, wherein objective response rate in the treated population ranges from 39% to 61%. In another embodiment, a population of the human subjects is treated by a method provided herein, wherein objective response rate in the treated population ranges from 39% to 62%. In another embodiment, a population of the human subjects is treated by a method provided herein, wherein objective response rate in the treated population ranges from 40% to 62%. In a further embodiment, a population of the human subjects is treated by a method provided herein, wherein objective response rate in the treated population ranges from 45% to 60%. In yet another embodiment, a population of the human subjects is treated by a method provided herein, wherein objective response rate in the treated population ranges from 50% to 55%. In one embodiment, a population of the human subjects is treated by a method provided herein, wherein objective response rate in the treated population ranges from 40% to 65%. In another embodiment, a population of the human subjects is treated by a method provided herein, wherein objective response rate in the treated population ranges from 45% to 65%. In a further embodiment, a population of the human subjects is treated by a method provided herein, wherein objective response rate in the treated population ranges from 50% to 65%. In yet another embodiment, a population of the human subjects is treated by a method provided herein, wherein objective response rate in the treated population ranges from 55% to 65%. In one embodiment, a population of the human subjects is treated by a method provided herein, wherein objective response rate in the treated population ranges from 60% to 65%. In another embodiment, a population of the human subjects is treated by a method provided herein, wherein objective response rate in the treated population ranges from 35% to 65%. In a further embodiment, a population of the human subjects is treated by a method provided herein, wherein objective response rate in the treated population ranges from 35% to 60%. In yet another embodiment, a population of the human subjects is treated by a method provided herein, wherein objective response rate in the treated population ranges from 35% to 55%. In one embodiment, a population of the human subjects is treated by a method provided herein, wherein objective response rate in the treated population ranges from 35% to 50%. In another embodiment, a population of the human subjects is treated by a method provided herein, wherein objective response rate in the treated population ranges from 35% to 45%. In a further embodiment, a population of the human subjects is treated by a method provided herein, wherein objective response rate in the treated population ranges from 35% to 40%.

Moreover, percentage of the subjects having stable disease can be used as the evaluation criteria for the therapeutic outcome in the human subjects treated by a method provided herein. In one embodiment, a population of the human subjects is treated by a method provided herein, wherein the percentage of the subjects having stable disease in the treated population is at least or about 10%. In one embodiment, a population of the human subjects is treated by a method provided herein, wherein the percentage of the subjects having stable disease in the treated population is at least or about 15%. In one embodiment, a population of the human subjects is treated by a method provided herein, wherein the percentage of the subjects having stable disease in the treated population is at least or about 20%. In one embodiment, a population of the human subjects is treated by a method provided herein, wherein the percentage of the subjects having stable disease in the treated population is at least or about 25%. In one embodiment, a population of the human subjects is treated by a method provided herein, wherein the percentage of the subjects having stable disease in the treated population is at least or about 30%. In one embodiment, a population of the human subjects is treated by a method provided herein, wherein the percentage of the subjects having stable disease in the treated population is at least or about 30.3%. In one embodiment, a population of the human subjects is treated by a method provided herein, wherein the percentage of the subjects having stable disease in the treated population is at least or about 35%. In one embodiment, a population of the human subjects is treated by a method provided herein, wherein the percentage of the subjects having stable disease in the treated population is at least or about 40%. In one embodiment, a population of the human subjects is treated by a method provided herein, wherein the percentage of the subjects having stable disease in the treated population is at least or about 45%. In one embodiment, a population of the human subjects is treated by a method provided herein, wherein the percentage of the subjects having stable disease in the treated population is at least or about 50%. In one embodiment, a population of the human subjects is treated by a method provided herein, wherein the percentage of the subjects having stable disease in the treated population is at least or about 55%. In one embodiment, a population of the human subjects is treated by a method provided herein, wherein the percentage of the subjects having stable disease in the treated population is at least or about 60%.

Additionally, the therapeutic outcome of the methods provided herein can be evaluated based on the duration of response as set forth in Section 6.1.8.2(ii). In one embodiment, the human subject has a duration of response of at least or about 5 months following the treatment. In a further embodiment, the human subject has a duration of response of at least or about 5.78 months following the treatment. In another embodiment, the human subject has a duration of response of at least or about 6 months following the treatment. In a further embodiment, the human subject has a duration of response of at least or about 7 months following the treatment. In yet another embodiment, the human subject has a duration of response of at least or about 8 months following the treatment. In one embodiment, the human subject has a duration of response of at least or about 9 months following the treatment. In another embodiment, the human subject has a duration of response of at least or about 10 months following the treatment. In a further embodiment, the human subject has a duration of response of at least or about 10.9 months following the treatment. In yet another embodiment, the human subject has a duration of response of at least or about 11 months following the treatment. In one embodiment, the human subject has a duration of response of at least or about 12 months following the treatment. In another embodiment, the human subject has a duration of response of at least or about 13 months following the treatment. In another embodiment, the human subject has a duration of response of at least or about 13.8 months following the treatment. In a further embodiment, the human subject has a duration of response of at least or about 14 months following the treatment. In yet another embodiment, the human subject has a duration of response of at least or about 15 months following the treatment. In one embodiment, the human subject has a duration of response of at least or about 16 months following the treatment. In another embodiment, the human subject has a duration of response of at least or about 17 months following the treatment. In a further embodiment, the human subject has a duration of response of at least or about 18 months following the treatment. In yet another embodiment, the human subject has a duration of response of at least or about 19 months following the treatment. In a further embodiment, the human subject has a duration of response of at least or about 20 months following the treatment.

In some embodiments, the human subject has a duration of response ranging from 5 to 22 following the treatment. In certain embodiments, the human subject has a duration of response ranging from 5.78 to 22 following the treatment. In certain embodiment, the human subject has a duration of response ranging from 5.78 to more than 22 following the treatment. In another embodiment, the human subject has a duration of response ranging from 5 to 21 months following the treatment. In a further embodiment, the human subject has a duration of response ranging from 5 to 20 months following the treatment. In yet another embodiment, the human subject has a duration of response ranging from 5 to 19 months following the treatment. In one embodiment, the human subject has a duration of response ranging from 5 to 18 months following the treatment. In another embodiment, the human subject has a duration of response ranging from 5 to 17 months following the treatment. In a further embodiment, the human subject has a duration of response ranging from 5 to 16 months following the treatment. In yet another embodiment, the human subject has a duration of response ranging from 5 to 15 months following the treatment. In one embodiment, the human subject has a duration of response ranging from 5 to 14 months following the treatment. In another embodiment, the human subject has a duration of response ranging from 5 to 13 months following the treatment. In a further embodiment, the human subject has a duration of response ranging from 5 to 12 months following the treatment. In yet another embodiment, the human subject has a duration of response ranging from 6 to 22 months following the treatment. In another embodiment, the human subject has a duration of response ranging from 6 to 21 months following the treatment. In a further embodiment, the human subject has a duration of response ranging from 6 to 20 months following the treatment. In yet another embodiment, the human subject has a duration of response ranging from 6 to 19 months following the treatment. In one embodiment, the human subject has a duration of response ranging from 6 to 18 months following the treatment. In another embodiment, the human subject has a duration of response ranging from 6 to 17 months following the treatment. In a further embodiment, the human subject has a duration of response ranging from 6 to 16 months following the treatment. In yet another embodiment, the human subject has a duration of response ranging from 6 to 15 months following the treatment. In one embodiment, the human subject has a duration of response ranging from 6 to 14 months following the treatment. In another embodiment, the human subject has a duration of response ranging from 6 to 13 months following the treatment. In a further embodiment, the human subject has a duration of response ranging from 6 to 12 months following the treatment. In one embodiment, the human subject has a duration of response ranging from 7 to 22 months following the treatment. In another embodiment, the human subject has a duration of response ranging from 7 to 21 months following the treatment. In a further embodiment, the human subject has a duration of response ranging from 7 to 20 months following the treatment. In yet another embodiment, the human subject has a duration of response ranging from 7 to 19 months following the treatment. In one embodiment, the human subject has a duration of response ranging from 7 to 18 months following the treatment. In another embodiment, the human subject has a duration of response ranging from 7 to 17 months following the treatment. In a further embodiment, the human subject has a duration of response ranging from 7 to 16 months following the treatment. In yet another embodiment, the human subject has a duration of response ranging from 7 to 15 months following the treatment. In one embodiment, the human subject has a duration of response ranging from 7 to 14 months following the treatment. In another embodiment, the human subject has a duration of response ranging from 7 to 13 months following the treatment. In a further embodiment, the human subject has a duration of response ranging from 7 to 12 months following the treatment. In another embodiment, the human subject has a duration of response ranging from 8 to 22 months following the treatment. In a further embodiment, the human subject has a duration of response ranging from 9 to 22 months following the treatment. In yet another embodiment, the human subject has a duration of response ranging from 10 to 22 months following the treatment. In a further embodiment, the human subject has a duration of response ranging from 11 to 22 months following the treatment. In one embodiment, the human subject has a duration of response ranging from 12 to 22 months following the treatment. In another embodiment, the human subject has a duration of response ranging from 13 to 22 months following the treatment. In a further embodiment, the human subject has a duration of response ranging from 14 to 22 months following the treatment. In yet another embodiment, the human subject has a duration of response ranging from 15 to 22 months following the treatment. In a further embodiment, the human subject has a duration of response ranging from 16 to 22 months following the treatment. In one embodiment, the human subject has a duration of response ranging from 17 to 22 months following the treatment. In another embodiment, the human subject has a duration of response ranging from 18 to 22 months following the treatment. In a further embodiment, the human subject has a duration of response ranging from 6 to 21 months following the treatment. In yet another embodiment, the human subject has a duration of response ranging from 7 to 20 months following the treatment. In a further embodiment, the human subject has a duration of response ranging from 8 to 19 months following the treatment. In one embodiment, the human subject has a duration of response ranging from 9 to 18 months following the treatment. In another embodiment, the human subject has a duration of response ranging from 10 to 17 months following the treatment. In a further embodiment, the human subject has a duration of response ranging from 11 to 16 months following the treatment. In yet another embodiment, the human subject has a duration of response ranging from 12 to 15 months following the treatment. In a further embodiment, the human subject has a duration of response ranging from 13 to 14 months following the treatment.

In some embodiments, the duration of response is evaluated for a population of human subjects treated by a method provided herein by evaluating the median or mean duration of response in the treated population. In one embodiment, a population of the human subjects is treated by a method provided herein, wherein the median or mean duration of response of in the treated population is at least or about 5 months. In some embodiments, a population of the human subjects is treated by a method provided herein, wherein the median or mean duration of response of in the treated population is at least or about 5.78 months. In another embodiment, a population of the human subjects is treated by a method provided herein, wherein the median or mean duration of response of in the treated population is at least or about 6 months. In a further embodiment, a population of the human subjects is treated by a method provided herein, wherein the median or mean duration of response of in the treated population is at least or about 7 months. In yet another embodiment, a population of the human subjects is treated by a method provided herein, wherein the median or mean duration of response of in the treated population is at least or about 8 months. In one embodiment, a population of the human subjects is treated by a method provided herein, wherein the median or mean duration of response of in the treated population is at least or about 9 months. In another embodiment, a population of the human subjects is treated by a method provided herein, wherein the median or mean duration of response of in the treated population is at least or about 10 months. In a further embodiment, a population of the human subjects is treated by a method provided herein, wherein the median or mean duration of response of in the treated population is at least or about 10.9 months. In yet another embodiment, a population of the human subjects is treated by a method provided herein, wherein the median or mean duration of response of in the treated population is at least or about 11 months. In one embodiment, a population of the human subjects is treated by a method provided herein, wherein the median or mean duration of response of in the treated population is at least or about 12 months. In another embodiment, a population of the human subjects is treated by a method provided herein, wherein the median or mean duration of response of in the treated population is at least or about 13 months. In one embodiment, a population of the human subjects is treated by a method provided herein, wherein the median or mean duration of response of in the treated population is at least or about 13.8 months. In a further embodiment, a population of the human subjects is treated by a method provided herein, wherein the median or mean duration of response of in the treated population is at least or about 14 months. In yet another embodiment, a population of the human subjects is treated by a method provided herein, wherein the median or mean duration of response of in the treated population is at least or about 15 months. In one embodiment, a population of the human subjects is treated by a method provided herein, wherein the median or mean duration of response of in the treated population is at least or about 16 months. In another embodiment, a population of the human subjects is treated by a method provided herein, wherein the median or mean duration of response of in the treated population is at least or about 17 months. In a further embodiment, a population of the human subjects is treated by a method provided herein, wherein the median or mean duration of response of in the treated population is at least or about 18 months. In yet another embodiment, a population of the human subjects is treated by a method provided herein, wherein the median or mean duration of response of in the treated population is at least or about 19 months. In one embodiment, a population of the human subjects is treated by a method provided herein, wherein the median or mean duration of response of in the treated population is at least or about 20 months.

In certain embodiment, a population of the human subjects is treated by a method provided herein, wherein the duration of response of in the treated population ranges from 5.78 to 22 months. In some embodiments, a population of the human subjects is treated by a method provided herein, wherein the duration of response of in the treated population ranges from 5.78 to more than 22 months. In one embodiment, a population of the human subjects is treated by a method provided herein, wherein the duration of response of in the treated population ranges from 5 to 22 months. In some embodiments, a population of the human subjects is treated by a method provided herein, wherein the duration of response of in the treated population ranges from 5 to 21 months. In another embodiment, a population of the human subjects is treated by a method provided herein, wherein the duration of response of in the treated population ranges from 5 to 20 months. In a further embodiment, a population of the human subjects is treated by a method provided herein, wherein the duration of response of in the treated population ranges from 5 to 19 months. In yet another embodiment, a population of the human subjects is treated by a method provided herein, wherein the duration of response of in the treated population ranges from 5 to 18 months. In one embodiment, a population of the human subjects is treated by a method provided herein, wherein the duration of response of in the treated population ranges from 5 to 17 months. In another embodiment, a population of the human subjects is treated by a method provided herein, wherein the duration of response of in the treated population ranges from 5 to 16 months. In a further embodiment, a population of the human subjects is treated by a method provided herein, wherein the duration of response of in the treated population ranges from 5 to 15 months. In yet another embodiment, a population of the human subjects is treated by a method provided herein, wherein the duration of response of in the treated population ranges from 5 to 14 months. In one embodiment, a population of the human subjects is treated by a method provided herein, wherein the duration of response of in the treated population ranges from 5 to 13 months. In another embodiment, a population of the human subjects is treated by a method provided herein, wherein the duration of response of in the treated population ranges from 5 to 12 months. In a further embodiment, a population of the human subjects is treated by a method provided herein, wherein the duration of response of in the treated population ranges from 6 to 22 months. In some embodiments, a population of the human subjects is treated by a method provided herein, wherein the duration of response of in the treated population ranges from 6 to 21 months. In another embodiment, a population of the human subjects is treated by a method provided herein, wherein the duration of response of in the treated population ranges from 6 to 20 months. In a further embodiment, a population of the human subjects is treated by a method provided herein, wherein the duration of response of in the treated population ranges from 6 to 19 months. In yet another embodiment, a population of the human subjects is treated by a method provided herein, wherein the duration of response of in the treated population ranges from 6 to 18 months. In one embodiment, a population of the human subjects is treated by a method provided herein, wherein the duration of response of in the treated population ranges from 6 to 17 months. In another embodiment, a population of the human subjects is treated by a method provided herein, wherein the duration of response of in the treated population ranges from 6 to 16 months. In a further embodiment, a population of the human subjects is treated by a method provided herein, wherein the duration of response of in the treated population ranges from 6 to 15 months. In yet another embodiment, a population of the human subjects is treated by a method provided herein, wherein the duration of response of in the treated population ranges from 6 to 14 months. In one embodiment, a population of the human subjects is treated by a method provided herein, wherein the duration of response of in the treated population ranges from 6 to 13 months. In another embodiment, a population of the human subjects is treated by a method provided herein, wherein the duration of response of in the treated population ranges from 6 to 12 months. In yet another embodiment, a population of the human subjects is treated by a method provided herein, wherein the duration of response of in the treated population ranges from 7 to 22 months. In some embodiments, a population of the human subjects is treated by a method provided herein, wherein the duration of response of in the treated population ranges from 7 to 21 months. In another embodiment, a population of the human subjects is treated by a method provided herein, wherein the duration of response of in the treated population ranges from 7 to 20 months. In a further embodiment, a population of the human subjects is treated by a method provided herein, wherein the duration of response of in the treated population ranges from 7 to 19 months. In yet another embodiment, a population of the human subjects is treated by a method provided herein, wherein the duration of response of in the treated population ranges from 7 to 18 months. In one embodiment, a population of the human subjects is treated by a method provided herein, wherein the duration of response of in the treated population ranges from 7 to 17 months. In another embodiment, a population of the human subjects is treated by a method provided herein, wherein the duration of response of in the treated population ranges from 7 to 16 months. In a further embodiment, a population of the human subjects is treated by a method provided herein, wherein the duration of response of in the treated population ranges from 7 to 15 months. In yet another embodiment, a population of the human subjects is treated by a method provided herein, wherein the duration of response of in the treated population ranges from 7 to 14 months. In one embodiment, a population of the human subjects is treated by a method provided herein, wherein the duration of response of in the treated population ranges from 7 to 13 months. In another embodiment, a population of the human subjects is treated by a method provided herein, wherein the duration of response of in the treated population ranges from 7 to 12 months. In one embodiment, a population of the human subjects is treated by a method provided herein, wherein the duration of response of in the treated population ranges from 6.41 to 22 months. In another embodiment, a population of the human subjects is treated by a method provided herein, wherein the duration of response of in the treated population ranges from 8 to 22 months. In a further embodiment, a population of the human subjects is treated by a method provided herein, wherein the duration of response of in the treated population ranges from 9 to 22 months. In yet another embodiment, a population of the human subjects is treated by a method provided herein, wherein the duration of response of in the treated population ranges from 10 to 22 months. In one embodiment, a population of the human subjects is treated by a method provided herein, wherein the duration of response of in the treated population ranges from 11 to 22 months. In another embodiment, a population of the human subjects is treated by a method provided herein, wherein the duration of response of in the treated population ranges from 12 to 12 months. In a further embodiment, a population of the human subjects is treated by a method provided herein, wherein the duration of response of in the treated population ranges from 13 to 22 months. In yet another embodiment, a population of the human subjects is treated by a method provided herein, wherein the duration of response of in the treated population ranges from 14 to 22 months. In one embodiment, a population of the human subjects is treated by a method provided herein, wherein the duration of response of in the treated population ranges from 15 to 22 months. In another embodiment, a population of the human subjects is treated by a method provided herein, wherein the duration of response of in the treated population ranges from 16 to 22 months. In a further embodiment, a population of the human subjects is treated by a method provided herein, wherein the duration of response of in the treated population ranges from 17 to 22 months. In yet another embodiment, a population of the human subjects is treated by a method provided herein, wherein the duration of response of in the treated population ranges from 18 to 22 months. In one embodiment, a population of the human subjects is treated by a method provided herein, wherein the duration of response of in the treated population ranges from 6 to 21 months. In another embodiment, a population of the human subjects is treated by a method provided herein, wherein the duration of response of in the treated population ranges from 7 to 20 months. In a further embodiment, a population of the human subjects is treated by a method provided herein, wherein the duration of response of in the treated population ranges from 8 to 19 months. In yet another embodiment, a population of the human subjects is treated by a method provided herein, wherein the duration of response of in the treated population ranges from 9 to 18 months. In one embodiment, a population of the human subjects is treated by a method provided herein, wherein the duration of response of in the treated population ranges from 10 to 17 months. In another embodiment, a population of the human subjects is treated by a method provided herein, wherein the duration of response of in the treated population ranges from 11 to 16 months. In a further embodiment, a population of the human subjects is treated by a method provided herein, wherein the duration of response of in the treated population ranges from 12 to 15 months. In yet another embodiment, a population of the human subjects is treated by a method provided herein, wherein the duration of response of in the treated population ranges from 13 to 24 months.

Alternatively, the therapeutic outcome of the methods provided herein can be evaluated based on the progression free survival as set forth in Section 6.1.8.2(iv). In one embodiment, the human subject has a progression free survival of at least or about 2 months following the treatment. In another embodiment, the human subject has a progression free survival of at least or about 3 months following the treatment. In a further embodiment, the human subject has a progression free survival of at least or about 4 months following the treatment. In yet another embodiment, the human subject has a progression free survival of at least or about 5 months following the treatment. In yet another embodiment, the human subject has a progression free survival of at least or about 5.03 months following the treatment. In one embodiment, the human subject has a progression free survival of at least or about 5.8 months following the treatment. In another embodiment, the human subject has a progression free survival of at least or about 6 months following the treatment. In one embodiment, the human subject has a progression free survival of at least or about 6.7 months following the treatment. In a further embodiment, the human subject has a progression free survival of at least or about 7 months following the treatment. In yet another embodiment, the human subject has a progression free survival of at least or about 8 months following the treatment. In one embodiment, the human subject has a progression free survival of at least or about 9 months following the treatment. In another embodiment, the human subject has a progression free survival of at least or about 10 months following the treatment. In a further embodiment, the human subject has a progression free survival of at least or about 11 months following the treatment. In yet another embodiment, the human subject has a progression free survival of at least or about 12 months following the treatment. In one embodiment, the human subject has a progression free survival of at least or about 13 months following the treatment. In another embodiment, the human subject has a progression free survival of at least or about 14 months following the treatment. In a further embodiment, the human subject has a progression free survival of at least or about 15 months following the treatment. In yet another embodiment, the human subject has a progression free survival of at least or about 16 months following the treatment. In one embodiment, the human subject has a progression free survival of at least or about 17 months following the treatment. In another embodiment, the human subject has a progression free survival of at least or about 18 months following the treatment. In a further embodiment, the human subject has a progression free survival of at least or about 19 months following the treatment. In yet another embodiment, the human subject has a progression free survival of at least or about 20 months following the treatment.

In one embodiment, the human subject has a progression free survival ranging from 5 to 10 months following the treatment. In some embodiments, the human subject has a progression free survival ranging from 5 to 9 months following the treatment. In another embodiment, the human subject has a progression free survival ranging from 5.03 to 8.28 months following the treatment. In one embodiment, the human subject has a progression free survival ranging from 5 to 8.3 months following the treatment. In a further embodiment, the human subject has a progression free survival ranging from 5 to 8 months following the treatment. In yet another embodiment, the human subject has a progression free survival ranging from 5 to 7 months following the treatment. In one embodiment, the human subject has a progression free survival ranging from 5 to 6 months following the treatment. In another embodiment, the human subject has a progression free survival ranging from 6 to 10 months following the treatment. In a further embodiment, the human subject has a progression free survival ranging from 7 to 10 months following the treatment. In yet another embodiment, the human subject has a progression free survival ranging from 8 to 10 months following the treatment. In one embodiment, the human subject has a progression free survival ranging from 9 to 10 months following the treatment. In another embodiment, the human subject has a progression free survival of ranging from 4 to 11 months following the treatment. In a further embodiment, the human subject has a progression free survival ranging from 4 to 10 months following the treatment. In yet another embodiment, the human subject has a progression free survival ranging from 4 to 9 months following the treatment. In one embodiment, the human subject has a progression free survival ranging from 4 to 8 months following the treatment. In another embodiment, the human subject has a progression free survival ranging from 4 to 7 months following the treatment. In a further embodiment, the human subject has a progression free survival ranging from 5 to 11 months following the treatment. In yet another embodiment, the human subject has a progression free survival ranging from 6 to 11 months following the treatment. In one embodiment, the human subject has a progression free survival ranging from 7 to 11 months following the treatment. In another embodiment, the human subject has a progression free survival ranging from 8 to 11 months following the treatment. In a further embodiment, the human subject has a progression free survival ranging from 9 to 11 months following the treatment. In yet another embodiment, the human subject has a progression free survival ranging from 10 to 11 months following the treatment.

In addition, in some embodiments, the progression free survival is evaluated for a population of human subjects treated by a method provided herein by evaluating the median or mean progression free survival in the treated population. In one embodiment, a population of the human subjects is treated by a method provided herein, wherein the median or mean progression free survival in the treated population is at least or about 2 months. In another embodiment, a population of the human subjects is treated by a method provided herein, wherein the median or mean progression free survival in the treated population is at least or about 3 months. In a further embodiment, a population of the human subjects is treated by a method provided herein, wherein the median or mean progression free survival in the treated population is at least or about 4 months. In yet another embodiment, a population of the human subjects is treated by a method provided herein, wherein the median or mean progression free survival in the treated population is at least or about 5 months. In yet another embodiment, a population of the human subjects is treated by a method provided herein, wherein the median or mean progression free survival in the treated population is at least or about 5.03 months. In one embodiment, a population of the human subjects is treated by a method provided herein, wherein the median or mean progression free survival in the treated population is at least or about 5.8 months. In another embodiment, a population of the human subjects is treated by a method provided herein, wherein the median or mean progression free survival in the treated population is at least or about 6 months. In another embodiment, a population of the human subjects is treated by a method provided herein, wherein the median or mean progression free survival in the treated population is at least or about 6.7 months. In a further embodiment, a population of the human subjects is treated by a method provided herein, wherein the median or mean progression free survival in the treated population is at least or about 7 months. In yet another embodiment, a population of the human subjects is treated by a method provided herein, wherein the median or mean progression free survival in the treated population is at least or about 8 months. In one embodiment, a population of the human subjects is treated by a method provided herein, wherein the median or mean progression free survival in the treated population is at least or about 9 months. In another embodiment, a population of the human subjects is treated by a method provided herein, wherein the median or mean progression free survival in the treated population is at least or about 10 months. In a further embodiment, a population of the human subjects is treated by a method provided herein, wherein the median or mean progression free survival in the treated population is at least or about 11 months. In yet another embodiment, a population of the human subjects is treated by a method provided herein, wherein the median or mean progression free survival in the treated population is at least or about 12 months. In one embodiment, a population of the human subjects is treated by a method provided herein, wherein the median or mean progression free survival in the treated population is at least or about 13 months. In another embodiment, a population of the human subjects is treated by a method provided herein, wherein the median or mean progression free survival in the treated population is at least or about 14 months. In a further embodiment, a population of the human subjects is treated by a method provided herein, wherein the median or mean progression free survival in the treated population is at least or about 15 months. In yet another embodiment, a population of the human subjects is treated by a method provided herein, wherein the median or mean progression free survival in the treated population is at least or about 16 months. In one embodiment, a population of the human subjects is treated by a method provided herein, wherein the median or mean progression free survival in the treated population is at least or about 17 months. In another embodiment, a population of the human subjects is treated by a method provided herein, wherein the median or mean progression free survival in the treated population is at least or about 18 months. In a further embodiment, a population of the human subjects is treated by a method provided herein, wherein the median or mean progression free survival in the treated population is at least or about 19 months. In yet another embodiment, a population of the human subjects is treated by a method provided herein, wherein the median or mean progression free survival in the treated population is at least or about 20 months.

In one embodiment, a population of the human subjects is treated by a method provided herein, wherein the progression free survival in the treated population ranges from 5 to 9 months. In some embodiments, a population of the human subjects is treated by a method provided herein, wherein the progression free survival in the treated population ranges from 5.03 to 8.28 months. In some embodiments, a population of the human subjects is treated by a method provided herein, wherein the progression free survival in the treated population ranges from 5 to 8.3 months. In another embodiment, a population of the human subjects is treated by a method provided herein, wherein the progression free survival in the treated population ranges from 5 to 8 months. In a further embodiment, a population of the human subjects is treated by a method provided herein, wherein the progression free survival in the treated population ranges from 5 to 7 months. In yet another embodiment, a population of the human subjects is treated by a method provided herein, wherein the progression free survival in the treated population ranges from 5 to 6 months. In one embodiment, a population of the human subjects is treated by a method provided herein, wherein the progression free survival in the treated population ranges from 6 to 9 months. In another embodiment, a population of the human subjects is treated by a method provided herein, wherein the progression free survival in the treated population ranges from 7 to 9 months. In a further embodiment, a population of the human subjects is treated by a method provided herein, wherein the progression free survival in the treated population ranges from 8 to 9 months. In yet another embodiment, a population of the human subjects is treated by a method provided herein, wherein the progression free survival in the treated population ranges from 4 to 10 months. In one embodiment, a population of the human subjects is treated by a method provided herein, wherein the progression free survival in the treated population ranges from 5 to 10 months. In another embodiment, a population of the human subjects is treated by a method provided herein, wherein the progression free survival in the treated population ranges from 6 to 10 months. In a further embodiment, a population of the human subjects is treated by a method provided herein, wherein the progression free survival in the treated population ranges from 7 to 10 months. In yet another embodiment, a population of the human subjects is treated by a method provided herein, wherein the progression free survival in the treated population ranges from 8 to 10 months. In one embodiment, a population of the human subjects is treated by a method provided herein, wherein the progression free survival in the treated population ranges from 9 to 10 months. In another embodiment, a population of the human subjects is treated by a method provided herein, wherein the progression free survival in the treated population ranges from 4 to 10 months. In another embodiment, a population of the human subjects is treated by a method provided herein, wherein the progression free survival in the treated population ranges from 4 to 9 months. In a further embodiment, a population of the human subjects is treated by a method provided herein, wherein the progression free survival in the treated population ranges from 4 to 8 months. In yet another embodiment, a population of the human subjects is treated by a method provided herein, wherein the progression free survival in the treated population ranges from 4 to 7 months. In one embodiment, a population of the human subjects is treated by a method provided herein, wherein the progression free survival in the treated population ranges from 4 to 6 months. In another embodiment, a population of the human subjects is treated by a method provided herein, wherein the progression free survival in the treated population ranges from 4 to 5 months. In a further embodiment, a population of the human subjects is treated by a method provided herein, wherein the progression free survival in the treated population ranges from 4 to 11 months. In yet another embodiment, a population of the human subjects is treated by a method provided herein, wherein the progression free survival in the treated population ranges from 5 to 11 months. In another embodiment, a population of the human subjects is treated by a method provided herein, wherein the progression free survival in the treated population ranges from 6 to 11 months. In a further embodiment, a population of the human subjects is treated by a method provided herein, wherein the progression free survival in the treated population ranges from 7 to 11 months. In yet another embodiment, a population of the human subjects is treated by a method provided herein, wherein the progression free survival in the treated population ranges from 8 to 11 months. In a further embodiment, a population of the human subjects is treated by a method provided herein, wherein the progression free survival in the treated population ranges from 9 to 11 months. In yet another embodiment, a population of the human subjects is treated by a method provided herein, wherein the progression free survival in the treated population ranges from 10 to 11 months.

Alternatively, the therapeutic outcome of the methods provided herein can be evaluated based on the overall survival as set forth in Section 6.1.8.2(v). In one embodiment, the human subject has an overall survival of at least or about 5 months following the treatment. In another embodiment, the human subject has an overall survival of at least or about 6 months following the treatment. In a further embodiment, the human subject has an overall survival of at least or about 7 months following the treatment. In yet another embodiment, the human subject has an overall survival of at least or about 8 months following the treatment. In one embodiment, the human subject has an overall survival of at least or about 9 months following the treatment. In another embodiment, the human subject has an overall survival of at least or about 10 months following the treatment. In another embodiment, the human subject has an overall survival of at least or about 10.51 months following the treatment. In a further embodiment, the human subject has an overall survival of at least or about 11 months following the treatment. In yet another embodiment, the human subject has an overall survival of at least or about 12 months following the treatment. In one embodiment, the human subject has an overall survival of at least or about 13 months following the treatment. In another embodiment, the human subject has an overall survival of at least or about 14 months following the treatment. In a further embodiment, the human subject has an overall survival of at least or about 14.7 months following the treatment. In yet another embodiment, the human subject has an overall survival of at least or about 15 months following the treatment. In one embodiment, the human subject has an overall survival of at least or about 16 months following the treatment. In one embodiment, the human subject has an overall survival of at least or about 16.1 months following the treatment. In another embodiment, the human subject has an overall survival of at least or about 17 months following the treatment. In a further embodiment, the human subject has an overall survival of at least or about 18 months following the treatment. In yet another embodiment, the human subject has an overall survival of at least or about 19 months following the treatment. In one embodiment, the human subject has an overall survival of at least or about 20 months following the treatment. In another embodiment, the human subject has an overall survival of at least or about 21 months following the treatment. In a further embodiment, the human subject has an overall survival of at least or about 22 months following the treatment. In a further embodiment, the human subject has an overall survival of at least or about 23 months following the treatment. In yet another embodiment, the human subject has an overall survival of at least or about 24 months following the treatment. In one embodiment, the human subject has an overall survival of at least or about 25 months following the treatment. In another embodiment, the human subject has an overall survival of at least or about 26 months following the treatment. In a further embodiment, the human subject has an overall survival of at least or about 27 months following the treatment. In one embodiment, the human subject has an overall survival of at least or about 28 months following the treatment. In another embodiment, the human subject has an overall survival of at least or about 29 months following the treatment. In a further embodiment, the human subject has an overall survival of at least or about 30 months following the treatment.

In one embodiment, the human subject has an overall survival ranging from 10 to 19 months following the treatment. In some embodiments, the human subject has an overall survival ranging from 10.51 to 18.20 months following the treatment. In another embodiment, the human subject has an overall survival ranging from 10 to 18 months following the treatment. In a further embodiment, the human subject has an overall survival ranging from 10 to 17 months following the treatment. In yet another embodiment, the human subject has an overall survival ranging from 10 to 16 months following the treatment. In one embodiment, the human subject has an overall survival ranging from 10 to 15 months following the treatment. In another embodiment, the human subject has an overall survival ranging from 10 to 14 months following the treatment. In a further embodiment, the human subject has an overall survival ranging from 10 to 13 months following the treatment. In yet another embodiment, the human subject has an overall survival ranging from 10 to 12 months following the treatment. In one embodiment, the human subject has an overall survival ranging from 10 to 11 months following the treatment. In another embodiment, the human subject has an overall survival ranging from 11 to 19 months following the treatment. In a further embodiment, the human subject has an overall survival ranging from 12 to 19 months following the treatment. In yet another embodiment, the human subject has an overall survival ranging from 13 to 19 months following the treatment. In one embodiment, the human subject has an overall survival ranging from 14 to 18 months following the treatment. In one embodiment, the human subject has an overall survival ranging from 14 to 19 months following the treatment. In one embodiment, the human subject has an overall survival ranging from 15 to 18 months following the treatment. In another embodiment, the human subject has an overall survival ranging from 15 to 19 months following the treatment. In a further embodiment, the human subject has an overall survival ranging from 16 to 19 months following the treatment. In yet another embodiment, the human subject has an overall survival ranging from 17 to 19 months following the treatment. In one embodiment, the human subject has an overall survival ranging from 18 to 19 months following the treatment. In another embodiment, the human subject has an overall survival ranging from 11 to 18 months following the treatment. In a further embodiment, the human subject has an overall survival ranging from 12 to 17 months following the treatment. In a further embodiment, the human subject has an overall survival ranging from 13 to 16 months following the treatment. In yet another embodiment, the human subject has an overall survival ranging from 14 to 15 months following the treatment. In one embodiment, the human subject has an overall survival ranging from 10 to 20 months following the treatment. In another embodiment, the human subject has an overall survival ranging from 11 to 20 months following the treatment. In one embodiment, the human subject has an overall survival ranging from 11 to 24 months following the treatment. In one embodiment, the human subject has an overall survival ranging from 11 to 25 months following the treatment. In one embodiment, the human subject has an overall survival ranging from 11.3 to 24.1 months following the treatment. In one embodiment, the human subject has an overall survival ranging from 12 to 24 months following the treatment. In one embodiment, the human subject has an overall survival ranging from 12 to 25 months following the treatment. In a further embodiment, the human subject has an overall survival ranging from 12 to 20 months following the treatment. In one embodiment, the human subject has an overall survival ranging from 13 to 20 months following the treatment. In another embodiment, the human subject has an overall survival ranging from 14 to 20 months following the treatment. In a further embodiment, the human subject has an overall survival ranging from 15 to 20 months following the treatment. In yet another embodiment, the human subject has an overall survival ranging from 16 to 20 months following the treatment. In one embodiment, the human subject has an overall survival ranging from 17 to 20 months following the treatment. In another embodiment, the human subject has an overall survival ranging from 18 to 20 months following the treatment. In a further embodiment, the human subject has an overall survival ranging from 19 to 20 months following the treatment. In one embodiment, the human subject has an overall survival ranging from 9 to 20 months following the treatment. In another embodiment, the human subject has an overall survival ranging from 9 to 19 months following the treatment. In a further embodiment, the human subject has an overall survival ranging from 9 to 18 months following the treatment. In one embodiment, the human subject has an overall survival ranging from 9 to 17 months following the treatment. In another embodiment, the human subject has an overall survival ranging from 9 to 16 months following the treatment. In a further embodiment, the human subject has an overall survival ranging from 9 to 15 months following the treatment. In one embodiment, the human subject has an overall survival ranging from 9 to 14 months following the treatment. In another embodiment, the human subject has an overall survival ranging from 9 to 13 months following the treatment. In a further embodiment, the human subject has an overall survival ranging from 9 to 12 months following the treatment. In one embodiment, the human subject has an overall survival ranging from 9 to 11 months following the treatment. In another embodiment, the human subject has an overall survival ranging from 9 to 10 months following the treatment.

Additionally, in some embodiments, the overall survival is evaluated for a population of human subjects treated by a method provided herein by evaluating the median or mean overall survival in the treated population. In one embodiment, a population of the human subjects is treated by a method provided herein, wherein the median or mean overall survival in the treated population is at least or about 5 months. In another embodiment, a population of the human subjects is treated by a method provided herein, wherein the median or mean overall survival in the treated population is at least or about 6 months. In a further embodiment, a population of the human subjects is treated by a method provided herein, wherein the median or mean overall survival in the treated population is at least or about 7 months. In yet another embodiment, a population of the human subjects is treated by a method provided herein, wherein the median or mean overall survival in the treated population is at least or about 8 months. In one embodiment, a population of the human subjects is treated by a method provided herein, wherein the median or mean overall survival in the treated population is at least or about 9 months. In another embodiment, a population of the human subjects is treated by a method provided herein, wherein the median or mean overall survival in the treated population is at least or about 10 months. In another embodiment, a population of the human subjects is treated by a method provided herein, wherein the median or mean overall survival in the treated population is at least or about 10.51 months. In a further embodiment, a population of the human subjects is treated by a method provided herein, wherein the median or mean overall survival in the treated population is at least or about 11 months. In yet another embodiment, a population of the human subjects is treated by a method provided herein, wherein the median or mean overall survival in the treated population is at least or about 12 months. In one embodiment, a population of the human subjects is treated by a method provided herein, wherein the median or mean overall survival in the treated population is at least or about 13 months. In another embodiment, a population of the human subjects is treated by a method provided herein, wherein the median or mean overall survival in the treated population is at least or about 14 months. In a further embodiment, a population of the human subjects is treated by a method provided herein, wherein the median or mean overall survival in the treated population is at least or about 14.7 months. In yet another embodiment, a population of the human subjects is treated by a method provided herein, wherein the median or mean overall survival in the treated population is at least or about 15 months. In one embodiment, a population of the human subjects is treated by a method provided herein, wherein the median or mean overall survival in the treated population is at least or about 16 months. In one embodiment, a population of the human subjects is treated by a method provided herein, wherein the median or mean overall survival in the treated population is at least or about 16.1 months. In another embodiment, a population of the human subjects is treated by a method provided herein, wherein the median or mean overall survival in the treated population is at least or about 17 months. In a further embodiment, a population of the human subjects is treated by a method provided herein, wherein the median or mean overall survival in the treated population is at least or about 18 months. In yet another embodiment, a population of the human subjects is treated by a method provided herein, wherein the median or mean overall survival in the treated population is at least or about 19 months. In one embodiment, a population of the human subjects is treated by a method provided herein, wherein the median or mean overall survival in the treated population is at least or about 20 months. In another embodiment, a population of the human subjects is treated by a method provided herein, wherein the median or mean overall survival in the treated population is at least or about 21 months. In a further embodiment, a population of the human subjects is treated by a method provided herein, wherein the median or mean overall survival in the treated population is at least or about 22 months. In yet another embodiment, a population of the human subjects is treated by a method provided herein, wherein the median or mean overall survival in the treated population is at least or about 23 months. In one embodiment, a population of the human subjects is treated by a method provided herein, wherein the median or mean overall survival in the treated population is at least or about 24 months. In another embodiment, a population of the human subjects is treated by a method provided herein, wherein the median or mean overall survival in the treated population is at least or about 25 months. In a further embodiment, a population of the human subjects is treated by a method provided herein, wherein the median or mean overall survival in the treated population is at least or about 26 months. In yet another embodiment, a population of the human subjects is treated by a method provided herein, wherein the median or mean overall survival in the treated population is at least or about 27 months. In one embodiment, a population of the human subjects is treated by a method provided herein, wherein the median or mean overall survival in the treated population is at least or about 28 months. In another embodiment, a population of the human subjects is treated by a method provided herein, wherein the median or mean overall survival in the treated population is at least or about 29 months. In a further embodiment, a population of the human subjects is treated by a method provided herein, wherein the median or mean overall survival in the treated population is at least or about 30 months.

In one embodiment, a population of the human subjects is treated by a method provided herein, wherein the overall survival in the treated population ranges from 10 to 19 months. In some embodiments, a population of the human subjects is treated by a method provided herein, wherein the overall survival in the treated population ranges from 10.51 to 18.2 months. In another embodiment, a population of the human subjects is treated by a method provided herein, wherein the overall survival in the treated population ranges from 10 to 18 months. In a further embodiment, a population of the human subjects is treated by a method provided herein, wherein the overall survival in the treated population ranges from 10 to 17 months. In yet another embodiment, a population of the human subjects is treated by a method provided herein, wherein the overall survival in the treated population ranges from 10 to 16 months. In one embodiment, a population of the human subjects is treated by a method provided herein, wherein the overall survival in the treated population ranges from 10 to 15 months. In another embodiment, a population of the human subjects is treated by a method provided herein, wherein the overall survival in the treated population ranges from 10 to 14 months. In a further embodiment, a population of the human subjects is treated by a method provided herein, wherein the overall survival in the treated population ranges from 10 to 13 months. In yet another embodiment, a population of the human subjects is treated by a method provided herein, wherein the overall survival in the treated population ranges from 10 to 12 months. In one embodiment, a population of the human subjects is treated by a method provided herein, wherein the overall survival in the treated population ranges from 10 to 11 months. In another embodiment, a population of the human subjects is treated by a method provided herein, wherein the overall survival in the treated population ranges from 10 to 19 months. In a further embodiment, a population of the human subjects is treated by a method provided herein, wherein the overall survival in the treated population ranges from 11 to 19 months. In one embodiment, a population of the human subjects is treated by a method provided herein, wherein the overall survival in the treated population ranges from 11 to 24 months. In a further embodiment, a population of the human subjects is treated by a method provided herein, wherein the overall survival in the treated population ranges from 11 to 25 months. In one embodiment, a population of the human subjects is treated by a method provided herein, wherein the overall survival in the treated population ranges from 11.3 to 24.1 months. In a further embodiment, a population of the human subjects is treated by a method provided herein, wherein the overall survival in the treated population ranges from 12 to 24 months. In one embodiment, a population of the human subjects is treated by a method provided herein, wherein the overall survival in the treated population ranges from 12 to 25 months. In yet another embodiment, a population of the human subjects is treated by a method provided herein, wherein the overall survival in the treated population ranges from 12 to 19 months. In one embodiment, a population of the human subjects is treated by a method provided herein, wherein the overall survival in the treated population ranges from 13 to 19 months. In another embodiment, a population of the human subjects is treated by a method provided herein, wherein the overall survival in the treated population ranges from 14 to 19 months. In a further embodiment, a population of the human subjects is treated by a method provided herein, wherein the overall survival in the treated population ranges from 15 to 19 months. In yet another embodiment, a population of the human subjects is treated by a method provided herein, wherein the overall survival in the treated population ranges from 16 to 19 months. In one embodiment, a population of the human subjects is treated by a method provided herein, wherein the overall survival in the treated population ranges from 17 to 19 months. In another embodiment, a population of the human subjects is treated by a method provided herein, wherein the overall survival in the treated population ranges from 18 to 19 months. In a further embodiment, a population of the human subjects is treated by a method provided herein, wherein the overall survival in the treated population ranges from 11 to 18 months. In yet another embodiment, a population of the human subjects is treated by a method provided herein, wherein the overall survival in the treated population ranges from 12 to 17 months. In one embodiment, a population of the human subjects is treated by a method provided herein, wherein the overall survival in the treated population ranges from 13 to 16 months. In another embodiment, a population of the human subjects is treated by a method provided herein, wherein the overall survival in the treated population ranges from 14 to 15 months. In a further embodiment, a population of the human subjects is treated by a method provided herein, wherein the overall survival in the treated population ranges from 10 to 20 months. In yet another embodiment, a population of the human subjects is treated by a method provided herein, wherein the overall survival in the treated population ranges from 11 to 20 months. In one embodiment, a population of the human subjects is treated by a method provided herein, wherein the overall survival in the treated population ranges from 12 to 20 months. In another embodiment, a population of the human subjects is treated by a method provided herein, wherein the overall survival in the treated population ranges from 13 to 20 months. In a further embodiment, a population of the human subjects is treated by a method provided herein, wherein the overall survival in the treated population ranges from 14 to 20 months. In yet another embodiment, a population of the human subjects is treated by a method provided herein, wherein the overall survival in the treated population ranges from 15 to 20 months. In one embodiment, a population of the human subjects is treated by a method provided herein, wherein the overall survival in the treated population ranges from 16 to 20 months. In another embodiment, a population of the human subjects is treated by a method provided herein, wherein the overall survival in the treated population ranges from 17 to 20 months. In a further embodiment, a population of the human subjects is treated by a method provided herein, wherein the overall survival in the treated population ranges from 18 to 20 months. In yet another embodiment, a population of the human subjects is treated by a method provided herein, wherein the overall survival in the treated population ranges from 19 to 20 months. In one embodiment, a population of the human subjects is treated by a method provided herein, wherein the overall survival in the treated population ranges from 9 to 20 months. In another embodiment, a population of the human subjects is treated by a method provided herein, wherein the overall survival in the treated population ranges from 9 to 19 months. In a further embodiment, a population of the human subjects is treated by a method provided herein, wherein the overall survival in the treated population ranges from 9 to 18 months. In yet another embodiment, a population of the human subjects is treated by a method provided herein, wherein the overall survival in the treated population ranges from 9 to 17 months. In one embodiment, a population of the human subjects is treated by a method provided herein, wherein the overall survival in the treated population ranges from 9 to 16 months. In another embodiment, a population of the human subjects is treated by a method provided herein, wherein the overall survival in the treated population ranges from 9 to 15 months. In a further embodiment, a population of the human subjects is treated by a method provided herein, wherein the overall survival in the treated population ranges from 9 to 14 months. In yet another embodiment, a population of the human subjects is treated by a method provided herein, wherein the overall survival in the treated population ranges from 9 to 13 months. In one embodiment, a population of the human subjects is treated by a method provided herein, wherein the overall survival in the treated population ranges from 9 to 12 months. In another embodiment, a population of the human subjects is treated by a method provided herein, wherein the overall survival in the treated population ranges from 9 to 11 months. In a further embodiment, a population of the human subjects is treated by a method provided herein, wherein the overall survival in the treated population ranges from 9 to 10 months.

In some embodiments, the human subjects and patients are used interchangeably. Therefore, a person skilled in the art would understand that the human subjects can be interchangeable with patients in any of the methods provided herein.

5.2.2 Methods of Treating Cancer in Patient Populations Based on Additional Selection Criteria

Provided herein are methods for the treatment of various cancers in subjects, wherein the cancers have any of the suitable markers and/or characteristics as provided in Section 6. Also provided herein are methods for the treatment of various cancers in subjects, wherein the subjects have any of the suitable characteristics as provided in Section 6.

In one aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the cancer has any of the suitable markers and/or characteristics as provided in Section 6.

In another aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the subject has any of the suitable characteristics as provided in Section 6. In a further aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the cancer has any of the suitable markers and/or characteristics as provided in Section 6.

In some embodiments of the methods provided herein including in Section 5.2 including Sections 5.2.1 and 5.2.2 and Sections 3 and 6, the subject is a human subject.

In all the methods provided herein and specifically those described in the Sections 5.2.1 and 5.2.2, the therapeutic agents including ADCs that can be used are described in Sections 3, 5.2, 5.3, 5.4, 5.5, and 6, selection of patients for treatment is described herein and exemplified in Section 5.2 including Sections 5.2.1 and 5.2.2 and Sections 3 and 6, dosing regimens and pharmaceutical composition for administering the therapeutic agent are described in Section 5.4, 5.6, 5.7, and Section 6 below, the biomarkers that can be used for identifying the therapeutic agents, selecting the patients, determining the outcome of these methods, and/or serving as criteria in any way for these methods are described herein and exemplified in Section 5.2 including Sections 5.2.1 and 5.2.2 and Sections 3 and 6, the biomarkers can be determined as described in Section 5.8 or as known in the art, therapeutic outcomes for the methods provided herein are described in this Section (Section 5.2 including Section 5.2.1.4) and Sections 3 and 6, additional therapeutic outcomes for the methods provided herein can be improvement of the biomarkers described herein, for example, those described and exemplified in in Section 5.2 including Sections 5.2.1 and 5.2.2 and Sections 3 and 6, and combination therapies including the ADCs and other therapeutic agents are described in this Section (Section 5.2) and in Section 5.5. Therefore, a person skilled in the art would understand that the methods provided herein include all permutations and combinations of the patients, therapeutic agents, dosing regiments, biomarkers, and therapeutic outcomes as described above and below.

### 5.3 Antibody Drug Conjugates for the Methods

In various embodiments of the methods provided herein, including the methods provided in Section 5.2, the ADC used in the methods comprises or is an anti-191P4D12 ADC described herein and/or in U.S. Pat. No. 8,637,642, which is herein incorporated in its entirety by reference. In some embodiments, the anti-191P4D12 antibody drug conjugate provided for the methods herein comprises an antibody or antigen binding fragment thereof that binds to 191P4D12 as provided herein, including in Sections 3, 5.3.1, and 6, conjugated to one or more units of cytotoxic agents (drug units, or D) as provided herein, including in Sections 3 and 6 and this Section (Section 5.3) with further disclosures in Sections 5.3.2 and 5.3.4. In certain embodiments, the cytotoxic agents (drug units, or D) can be covalently linked directly or via a linker unit (LU) as provided herein, including in Sections 3 and 6 and this Section (Section 5.3) with further disclosures in Section 5.3.3.

In some embodiments, the antibody drug conjugate compound has the following formula:

L-(LU-D)ₚ (I)
or a pharmaceutically acceptable salt or solvate thereof; wherein:
L is the antibody unit, e.g., the anti-nectin-4 antibody or an antigen binding fragment thereof for example as provided in Sections 3, 5.3.1, and 6, and
(LU-D) is a linker unit-drug unit moiety, wherein:
LU- is a linker unit for example as provided in Sections 3 and 6 and this Section (Section 5.3) with further disclosures in Section 5.3.3, and
D is a drug unit having cytostatic or cytotoxic activity against a target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{W}-Y_{y}-D)ₚ (II)
or a pharmaceutically acceptable salt or solvate thereof, wherein:
L is the Antibody unit, e.g., the anti-nectin-4 antibody or an antigen binding fragment thereof for example as provided in Sections 3, 5.3.1, and 6; and
Aₐ-W_{w}-Y_{y}- is a linker unit (LU), wherein:
-A- is a stretcher unit,
a is 0 or 1,
each -W- is independently an amino acid unit,
w is an integer ranging from 0 to 12,
-Y- is a self-immolative spacer unit,
y is 0, 1 or 2,
each for example as provided in Sections 3 and 6 and this Section (Section 5.3) with further disclosures in Section 5.3.3;
D is a drug units having cytostatic or cytotoxic activity against the target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In some specific embodiments of the methods provided herein, including the methods provided in Section 5.2, the cytotoxic agent as part of any of the ADCs provided herein for the methods comprises, consists of, or is MMAE.

For compositions comprising a plurality antibodies or antigen binding fragments thereof, the drug loading is represented by p, the average number of drug molecules per antibody unit. Drug loading can range from 1 to 20 drugs (D) per antibody. The average number of drugs per antibody in preparation of conjugation reactions can be characterized by conventional means such as mass spectroscopy, ELISA assay, and HPLC. The quantitative distribution of antibody drug conjugates in terms of p can also be determined. In some instances, separation, purification, and characterization of homogeneous antibody drug conjugates where p is a certain value from antibody drug conjugates with other drug loadings can be achieved by means such as reverse phase HPLC or electrophoresis. In certain exemplary embodiments, p is from 2 to 8.

Additional embodiments of the ADC for the methods provided herein have been described in U.S. Pat. No. 8,637,642 and International Application No. PCT/US2019/056214 (Publication No. WO2020/117373), both of which are hereby incorporated in their entireties by reference.

In some embodiments of the methods provided herein, including in Sections 3, 5.2, and 6 and this Section (Section 5.3), the ADC is enfortumab vedotin. In certain embodiments of the methods provided herein, including in Sections 3, 5.2, and 6 and this Section (Section 5.3), the ADC is a biosimilar of enfortumab vedotin.

5.3.1 Anti-191P4D12 Antibodies or Antigen Binding Fragments

In one embodiment, the antibody or antigen binding fragment thereof that binds to nectin-4-related proteins is an antibody or antigen binding fragment that specifically binds to nectin-4 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to nectin-4 protein comprising amino acid sequence of SEQ ID NO:2 includes antibodies that can bind to other nectin-4-related proteins. For example, antibodies that bind nectin-4 protein comprising amino acid sequence of SEQ ID NO:2 can bind nectin-4-related proteins such as nectin-4 variants and the homologs or analogs thereof.

In some embodiments, the anti-nectin-4 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO:6 (cDNA sequence of SEQ ID NO:5), as shown in FIGS. 1B and 1C.

In some embodiments, the anti-nectin-4 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). In some embodiments, the anti-nectin-4 antibody or antigen binding fragment thereof comprises a heavy chain variable region comprising complementarity determining regions (CDRs) consisting of the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs consisting of the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). SEQ ID NO:22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMNWVRQAPGKGLEWVS
YISSSSSTIYYADSVKGRFTISRDNAKNSLSLQMNSLRDEDTAVYYCAR
AYYYGMDVWGQGTTVTVSS
SEQ ID NO: 23
DIQMTQSPSSVSASVGDRVTITCRASQGISGWLAWYQQKPGKAPKFLIY
AASTLQSGVPSRFSGSGSGTDFTLTISSLQPEDFATYYCQQANSFPPTF
GGGTKVEIKR
SEQ ID NO: 7
MELGLCWVFLVAILEGVQCEVQLVESGGGLVQPGGSLRLSCAASGFTFS
SYNMNWVRQAPGKGLEWVSYISSSSSTIYYADSVKGRFTISRDNAKNSL
SLQMNSLRDEDTAVYYCARAYYYGMDVWGQGTTVTVSSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK
SEQ ID NO: 8
MDMRVPAQLLGLLLLWFPGSRCDIQMTQSPSSVSASVGDRVTITCRASQ
GISGWLAWYQQKPGKAPKFLIYAASTLQSGVPSRFSGSGSGTDFTLTIS
SLQPEDFATYYCQQANSFPPTFGGGTKVEIKRTVAAPSVFIFPPSDEQL
KSGTASVVCLLNNFYPREAKVQWKVDNALQSGNSQESVTEQDSKDSTYS
LSSTLTLSKADYEKHKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see. e.g., Kabat et al., supra). Chothia refers instead to the location of the structural loops (see. e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309; 657-70. Correspondence between the numbering system, including, for example, the Kabat numbering and the IMGT unique numbering system, is well-known to one skilled in the art (see. e.g., Kabat, supra; Chothia and Lesk, supra; Martin, supra; Lefranc et al., supra). The residues from each of these hypervariable regions or CDRs are noted in Table 1 above.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2. CDR-H3, CDR-L1, CDR-L2, and CDR-L3) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Kabat numbering and a light chain variable region comprising CDRs consisting of the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to AbM numbering and a light chain variable region comprising CDRs consisting of the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Chothia numbering and a light chain variable region comprising CDRs consisting of the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Contact numbering and a light chain variable region comprising CDRs consisting of the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to IMGT numbering and a light chain variable region comprising CDRs consisting of the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:22, and the light chain CDR sequences within SEQ ID NO:23 according to Kabat numbering as determined by ANARCI are listed in Table 4 below.

TABLE 4

| | VH of SEQ ID NO: 22 | VL of SEQ ID NO: 23 |
|---|---|---|
| CDR1 | SYNMN (SEQ ID NO: 9) | RASQGISGWLA (SEQ ID NO: 12) |
| CDR2 | YISSSSSTIYYADSVKG (SEQ ID NO: 10) | AASTLQS (SEQ ID NO: 13) |
| CDR3 | AYYYGMDV (SEQ ID NO: 11) | QQANSFPPT (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 5 below.

TABLE 5

| | VH of SEQ ID NO: 22 | VL of SEQ ID NO: 23 |
|---|---|---|
| CDR1 | GFTFSSYN (SEQ ID NO: 16) | QGISGW (SEQ ID NO: 19) |
| CDR2 | ISSSSSTI (SEQ ID NO: 17) | AAS (SEQ ID NO: 20) |
| CDR3 | ARAYYYGMDV (SEQ ID NO: 18) | QQANSFPPT (SEQ ID NO: 21) |

In some embodiments, the antibody or antigen binding fragment thereof comprises CDR-H1 comprising an amino acid sequence of SEQ ID NO:9, CDR-H2 comprising an amino acid sequence of SEQ ID NO:10, CDR-H3 comprising an amino acid sequence of SEQ ID NO:11, CDR-L1 comprising an amino acid sequence of SEQ ID NO:12, CDR-L2 comprising an amino acid sequence of SEQ ID NO:13, and CDR-L3 comprising an amino acid sequence of SEQ ID NO:14.

In some embodiments, the antibody or antigen binding fragment thereof comprises CDR-H1 comprising an amino acid sequence of SEQ ID NO:16, CDR-H2 comprising an amino acid sequence of SEQ ID NO:17, CDR-H3 comprising an amino acid sequence of SEQ ID NO: 18, CDR-L1 comprising an amino acid sequence of SEQ ID NO:19, CDR-L2 comprising an amino acid sequence of SEQ ID NO:20, and CDR-L3 comprising an amino acid sequence of SEQ ID NO:21.

In some embodiments, the antibody or antigen binding fragment thereof comprises CDR-H1 consisting of an amino acid sequence of SEQ ID NO:9, CDR-H2 consisting of an amino acid sequence of SEQ ID NO:10. CDR-H3 consisting of an amino acid sequence of SEQ ID NO:11, CDR-L1 consisting of an amino acid sequence of SEQ ID NO:12, CDR-L2 consisting of an amino acid sequence of SEQ ID NO:13, and CDR-L3 consisting of an amino acid sequence of SEQ ID NO:14.

In some embodiments, the antibody or antigen binding fragment thereof comprises CDR-H1 consisting of an amino acid sequence of SEQ ID NO:16, CDR-H2 consisting of an amino acid sequence of SEQ ID NO:17, CDR-H3 consisting of an amino acid sequence of SEQ ID NO:18, CDR-L1 consisting of an amino acid sequence of SEQ ID NO:19, CDR-L2 consisting of an amino acid sequence of SEQ ID NO:20, and CDR-L3 consisting of an amino acid sequence of SEQ ID NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes can alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives can include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formulation, metabolic synthesis of tunicamycin, etc. Additionally, the antibody can contain one or more non-classical amino acids.

Variations can be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions can optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed can be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions can be made, so as to maintain or not significantly change the properties.

Amino acids can be grouped according to similarities in the properties of their side chains (see, e.g., Lehninger, Biochemistry 73-75 (2d ed. 1975)): (1) non-polar: Ala (A). Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also can be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see. e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see. e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In certain embodiments, the antibody or antigen binding fragment provided herein comprises a heavy chain having certain homology or identity to the heavy chain as set forth in SEQ ID NO:7 and a light chain having certain homology or identity to the light chain as set forth in SEQ ID NO:8. Such embodiments of heavy/light chains with homology or identity are further provided as follows. In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7. In certain embodiments, the antibody or antigen binding fragment provided herein comprises a heavy chain having any of the provided homology or identity to the heavy chain as set forth in SEQ ID NO:7, wherein the CDRs (CDR-H1. CDR-H2, and CDR-H3) are identical to the CDRs in the heavy chain as set forth in SEQ ID NO:7. In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8. In certain embodiments, the antibody or antigen binding fragment provided herein comprises a light chain having any of the provided homology or identity to the light chain as set forth in SEQ ID NO:8, wherein the CDRs (CDR-L1, CDR-L2, and CDR-L3) are identical to the CDRs in the light chain as set forth in SEQ ID NO:8. In certain embodiments, the antibody or antigen binding fragment provided herein comprises any homologous light chain and any homologous heavy chain as provided in this paragraph in any combination or permutation.

In certain embodiments, the antibody or antigen binding fragment provided herein comprises a heavy chain variable region having certain homology or identity to the heavy chain variable region as set forth in SEQ ID NO:22 and a light chain variable region having certain homology or identity to the light chain variable region as set forth in SEQ ID NO:23. Such embodiments of heavy chain variable regions and light chain variable regions with homology or identity are further provided as follows. In some embodiments, the antibody or antigen binding fragment provided herein comprises a heavy chain variable region having more than 70% homology or identity to the heavy chain variable region as set forth in SEQ ID NO:22. In some embodiments, the antibody or antigen binding fragment provided herein comprises a heavy chain variable region having more than 75% homology or identity to the heavy chain variable region as set forth in SEQ ID NO:22. In some embodiments, the antibody or antigen binding fragment provided herein comprises a heavy chain variable region having more than 80% homology or identity to the heavy chain variable region as set forth in SEQ ID NO:22. In some embodiments, the antibody or antigen binding fragment provided herein comprises a heavy chain variable region having more than 85% homology or identity to the heavy chain variable region as set forth in SEQ ID NO:22. In some embodiments, the antibody or antigen binding fragment provided herein comprises a heavy chain variable region having more than 90% homology or identity to the heavy chain variable region as set forth in SEQ ID NO:22. In some embodiments, the antibody or antigen binding fragment provided herein comprises a heavy chain variable region having more than 95% homology or identity to the heavy chain variable region as set forth in SEQ ID NO:22. In certain embodiments, the antibody or antigen binding fragment provided herein comprises a heavy chain variable region having any of the provided homology or identity to the heavy chain variable region as set forth in SEQ ID NO:22, wherein the CDRs (CDR-H1, CDR-H2, and CDR-H3) are identical to the CDRs in the heavy chain variable region as set forth in SEQ ID NO:22. In some embodiments, the antibody or antigen binding fragment provided herein comprises a light chain variable region having more than 70% homology or identity to the light chain variable region as set forth in SEQ ID NO:23. In some embodiments, the antibody or antigen binding fragment provided herein comprises a light chain variable region having more than 75% homology or identity to the light chain variable region as set forth in SEQ ID NO:23. In some embodiments, the antibody or antigen binding fragment provided herein comprises a light chain variable region having more than 80% homology or identity to the light chain variable region as set forth in SEQ ID NO:23. In some embodiments, the antibody or antigen binding fragment provided herein comprises a light chain variable region having more than 85% homology or identity to the light chain variable region as set forth in SEQ ID NO:23. In some embodiments, the antibody or antigen binding fragment provided herein comprises a light chain variable region having more than 90% homology or identity to the light chain variable region as set forth in SEQ ID NO:23. In some embodiments, the antibody or antigen binding fragment provided herein comprises a light chain variable region having more than 95% homology or identity to the light chain variable region as set forth in SEQ ID NO:23. In certain embodiments, the antibody or antigen binding fragment provided herein comprises a light chain variable region having any of the provided homology or identity to the light chain variable region as set forth in SEQ ID NO:23, wherein the CDRs (CDR-L1, CDR-L2, and CDR-L3) are identical to the CDRs in the light chain variable region as set forth in SEQ ID NO:23. In certain embodiments, the antibody or antigen binding fragment provided herein comprises any homologous light chain variable region and any homologous heavy chain variable region as provided in this paragraph in any combination or permutation.

In some embodiments, the anti-nectin-4 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the anti-nectin-4 antibody provided herein comprises heavy and light chain CDR regions (CDR-H1, CDR-H2, CDR-H3, CDR-LL, CDR-L2, and CDR-L3) of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) comprising the amino acid sequences of the heavy chain variable region CDRs set forth in the antibody produced by a hybridoma deposited under the American Type Culture Collection (ATCC) Accession NO: PTA-11267:
(b) the light chain variable region comprises CDRs (CDR-L1, CDR-L2, and CDR-L3) comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) consisting of the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) consisting of the amino acid sequences of the light chain variable region CDRs set forth in the antibody produced by a hybridoma deposited under the American Type Culture Collection (ATCC) Accession NO: PTA-11267.

In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions comprising amino acid sequences that are homologous to the amino acid sequences of the heavy and light chain variable regions of Ha22-2(2,4)6.1, and wherein the antibodies retain the desired functional properties of the anti-nectin-4 antibody provided herein. In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions consisting of amino acid sequences that are homologous to the amino acid sequences of the heavy and light chain variable regions of Ha22-2(2,4)6.1, and wherein the antibodies retain the desired functional properties of the anti-nectin-4 antibody provided herein. As the constant region of the antibody of the disclosure, any subclass of constant region can be chosen. In one embodiment, human IgG1 constant region as the heavy chain constant region and human Ig kappa constant region as the light chain constant region can be used.

In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains comprising amino acid sequences that are homologous to the amino acid sequences of the heavy and light chains of Ha22-2(2,4)6.1, and wherein the antibodies retain the desired functional properties of the anti-nectin-4 antibody provided herein. In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains consisting of amino acid sequences that are homologous to the amino acid sequences of the heavy and light chains of Ha22-2(2,4)6.1, and wherein the antibodies retain the desired functional properties of the anti-nectin-4 antibody provided herein.

In some embodiments, the antibody or antigen binding fragment thereof provided herein comprises a heavy chain variable region and a light chain variable region, wherein:
(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-1267; and
(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In certain embodiments, the antibody or antigen binding fragment provided herein comprises a heavy chain variable region having certain homology or identity to the heavy chain variable region amino acid sequence of the antibody produced by a hybridoma deposited under the American Type Culture Collection (ATCC) Accession NO: PTA-11267 and a light chain variable region having certain homology or identity to the light chain variable region amino acid sequence of the antibody produced by a hybridoma deposited under the American Type Culture Collection (ATCC) Accession NO: PTA-11267. Such embodiments of heavy chain variable regions and light chain variable regions with homology or identity are further provided as follows. In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region can be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267. In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region can be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267. In certain embodiments, the antibody or antigen binding fragment provided herein comprises any homologous light chain variable region and any homologous heavy chain variable region as provided in this paragraph in any combination or permutation.

In other embodiments, the antibody or antigen binding fragment thereof provided herein comprises a heavy chain and a light chain, wherein:
(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and
(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In certain embodiments, the antibody or antigen binding fragment provided herein comprises a heavy chain having certain homology or identity to the heavy chain amino acid sequence of the antibody produced by a hybridoma deposited under the American Type Culture Collection (ATCC) Accession NO: PTA-11267 and a light chain having certain homology or identity to the light chain amino acid sequence of the antibody produced by a hybridoma deposited under the American Type Culture Collection (ATCC) Accession NO: PTA-11267. Such embodiments of heavy chains and light chains with homology or identity are further provided as follows. In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain can be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267. In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain can be 85%, 86%, 87%, 88%, 89%, 90%, 91%, 92%, 93%, 94%, 95%, 96%, 97%, 98% or 990% homologous or identical to the light chain amino acid sequence of the antibody produced by a hybridoma deposited under the American Type Culture Collection (ATCC) Accession NO: PTA-11267. In certain embodiments, the antibody or antigen binding fragment provided herein comprises any homologous light chain and any homologous heavy chain as provided in this paragraph in any combination or permutation.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 11 Ith to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphism observed in human. In some embodiments, the antibody or antigen binding fragment thereof provided herein binds to epitopes in 191P4D12 that are common between the 191P4D12 polymorphism observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the disclosure.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein can be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, VA 20108 on 18 Aug. 2010 and assigned Accession number PTA-11267.

Additional embodiments of anti-nectin-4 antibody have been described in U.S. Pat. No. 8,637,642 and International Application No. PCT/US2019/056214 (Publication No. WO2020/117373), both of which are hereby incorporated in their entireties by reference.

5.3.2 Cytotoxic Agents (Drug Units)

As the ADC used in the methods provided herein comprises an antibody or antigen binding fragment thereof conjugated to a cytotoxic agent, the disclosure further provides various embodiments for the cytotoxic agent as part of the ADC for use in the methods. In various embodiments of the methods provided herein, including the methods provided in Section 5.2, the cytotoxic agent as part of any of the ADCs provided herein for the methods comprises, consists of, or is a tubulin disrupting agent. In one embodiment, the cytotoxic agent is a tubulin disrupting agent. In some embodiments, the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane. In one specific embodiment, the tubulin disrupting agent is an auristatin. In a further specific embodiment, the auristatin is monomethyl auristatin E (MMAE), monomethyl auristatin F (MMAF), AFP, or auristain T. In yet another specific embodiment, the auristatin is monomethyl auristatin E (MMAE).

In various embodiments of the methods provided herein, including the methods provided in Section 5.2, the cytotoxic agent as part of any of the ADCs provided herein for the methods comprises, consists of, or is any agent selected from the cytotoxic agents described in U.S. Pat. No. 8,637,642 and International Application No. PCT/US2019/056214 (Publication No. WO2020/117373), both of which are hereby incorporated in their entireties by reference.

In some embodiments, the auristatin is MMAE (wherein the wavy line indicates the covalent attachment to a linker of an antibody drug conjugate).

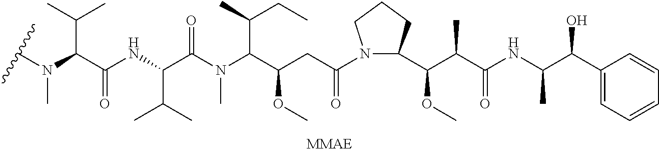 [SMILES: *N(C)[C@H](C(=O)N[C@H](C(=O)N(C)C([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody (e.g. anti-nectin-4 antibody or antigen binding fragment thereof) and p ranges from 1 to 12);

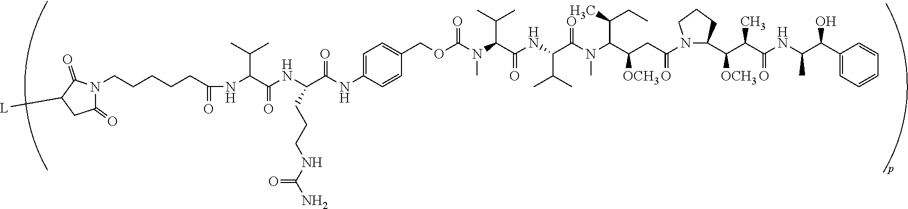

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schroder and K. Lübke, “The Peptides”, volume 1, pp 76-136, 1965, Academic Press) that is well-known in the field of peptide chemistry. The auristatin/dolastatin drug units can be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

Additional embodiments of cytotoxic agent have been described in U.S. Pat. No. 8,637,642 and International Application No. PCT/US2019/056214 (Publication No. WO2020/117373), both of which are hereby incorporated in their entireties by reference.

5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO: 15)). In some embodiments, the peptidyl linker is at least two amino acids long or at least three amino acids long. In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT.

A “linker unit” (LU) is a bifunctional compound that can be used to link a drug unit and an antibody unit to form an antibody drug conjugate. In some embodiments, the linker unit has the formula:

-Aₐ-W_{w}-Y_{y}-
wherein: -A- is a stretcher unit,
a is 0 or 1,
each -W- is independently an amino acid unit,
w is an integer ranging from 0 to 12,
-Y- is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0. The linker and each of the stretcher unit, the amino acid unit, and the spacer unit have been described in U.S. Pat. No. 8,637,642 and International Application No. PCT/US2019/056214 (Publication No. WO2020/117373), both of which are hereby incorporated in their entireties by reference.

Embodiments of the antibody-drug conjugates can include:

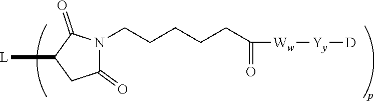

wherein w and v are each 0, 1 or 2, and,

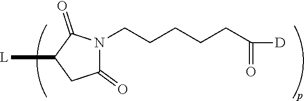

wherein w and y are each 0,

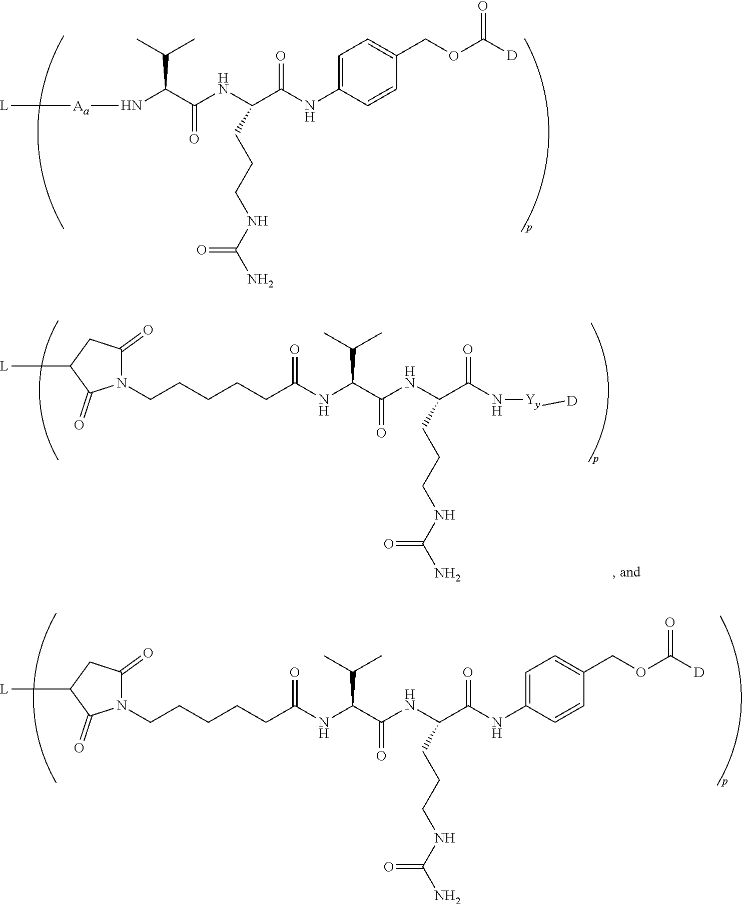

Drug loading is represented by p and is the average number of drug units per antibody in a molecule. Drug loading can range from 1 to 20 drug units (D) per antibody, the ADCs provided herein include collections of antibodies or antigen binding fragments conjugated with a range of drug units, e.g., from 1 to 20. The average number of drug units per antibody in preparations of ADC from conjugation reactions can be characterized by conventional means such as mass spectroscopy and, ELISA assay. The quantitative distribution of ADC in terms of p can also be determined. In some instances, separation, purification, and characterization of homogeneous ADC where p is a certain value from ADC with other drug loadings can be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody can contain, for example, lysine residues that do not react with the drug-linker intermediate or linker reagent. Generally, antibodies do not contain many free and reactive cysteine thiol groups which can be linked to a drug unit; indeed most cysteine thiol residues in antibodies exist as disulfide bridges. In certain embodiments, an antibody can be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC can be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody can be calculated from the mixture by a dual ELISA antibody assay, which is specific for antibody and specific for the drug. Individual ADC molecules can be identified in the mixture by mass spectroscopy and separated by HPLC, e.g. hydrophobic interaction chromatography (see. e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value can be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

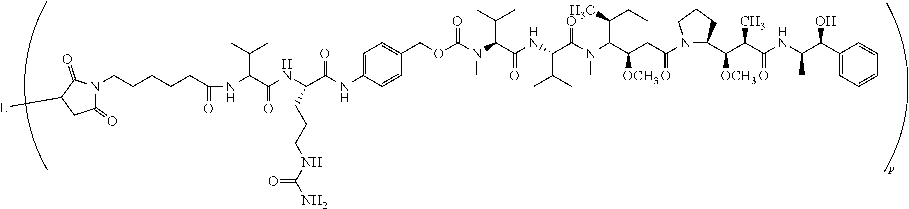

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1_{K}) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15:102(4):1458-65).

As described above and in in U.S. Pat. No. 8,637,642, AGS-22M6E is an ADC derived from a murine hybridoma cell line. Enfortumab vedotin is a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In one embodiment, the ADC provided herein is enfortumab vedotin, also known as EV, PADCEV, AGS-22M6E, AGS-22C3E, ASG-22C3E. The enfortumab vedotin includes an anti-191P4D12 antibody, wherein the antibody or antigen binding fragment thereof comprises a heavy chain comprising amino acid residue 20 to amino acid residue 466 of SEQ ID NO:7 and a light chain comprising amino acid residue 23 to amino acid residue 236 of SEQ ID NO:8.

Enfortumab vedotin is a Nectin-4 directed antibody-drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin has the following structural formula:

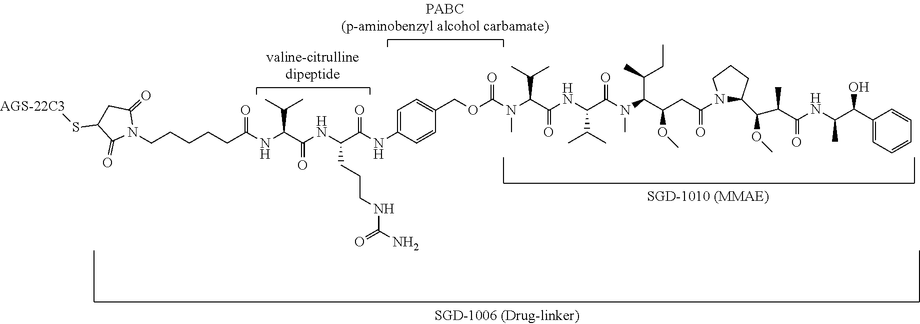

Approximately 4 molecules of MMAE are attached to each antibody molecule. Enfortumab vedotin is produced by chemical conjugation of the antibody and small molecule components. The antibody is produced by mammalian (Chinese hamster ovary) cells and the small molecule components are produced by chemical synthesis.

Enfortumab vedotin injection is provided as a sterile, preservative-free, white to off-white lyophilized powder in single-dose vials for intravenous use. Enfortumab vedotin is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL. After reconstitution, each vial allows the withdrawal of 2 mL (20 mg) and 3 mL (30 mg). Each mL of reconstituted solution contains 10 mg of enfortumab vedotin, histidine (1.4 mg), histidine hydrochloride monohydrate (2.31 mg), polysorbate 20 (0.2 mg) and trehalose dihydrate (55 mg) with a pH of 6.0.

### 5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs can further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds can be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 above.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises

L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM.

In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10°/o (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl. and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises
(a) an antibody drug conjugate comprising the following structure:

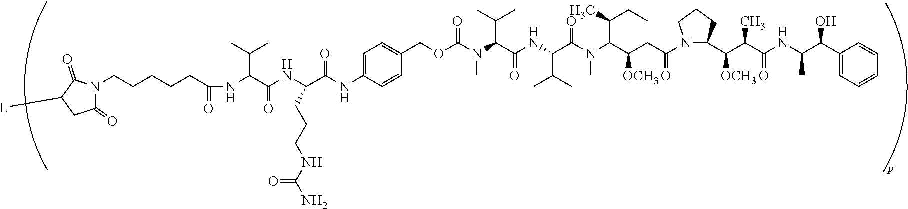

wherein L- represents the antibody or antigen binding fragment (e.g. anti-nectin-4 antibody or antigen binding fragment thereof) thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

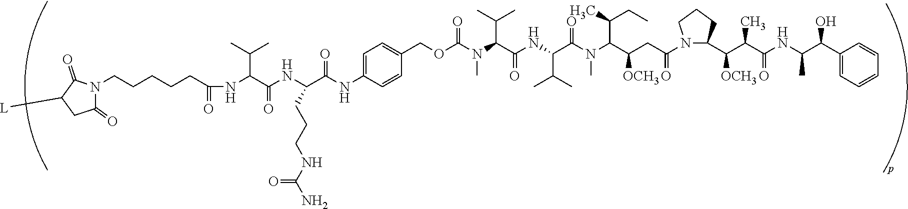

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure.

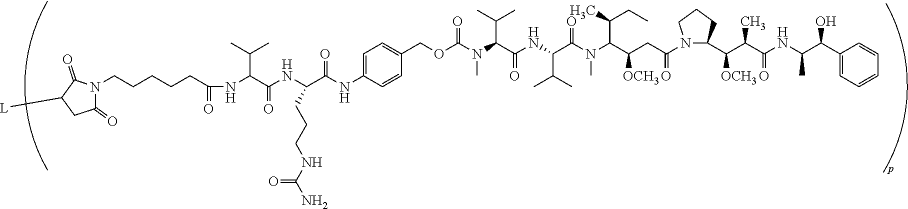

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated.

A primary solvent in a vehicle can be either aqueous or non-aqueous in nature. In addition, the vehicle can contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein can contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions can be in the form of a sterile injectable aqueous or oleagenous suspension. This suspension can be formulated using suitable dispersing or wetting agents and suspending agents disclosed herein or known to the skilled artisan. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see. e.g., Remington, The Science and Practice of Pharmacy, supra).

In one embodiment, the pharmaceutical compositions intended for parenteral administration can include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, KS).

In one embodiment, the pharmaceutical compositions provided herein can be formulated for single or multiple dosage administration. The single dosage formulations are packaged in an ampoule, a vial, or a syringe. The multiple dosage parenteral formulations can contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein can be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein can be stored at −80° C., 4° C., 25° C., or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They can also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C., or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

Additional embodiments for the pharmaceutical compositions have been described in U.S. Pat. No. 8,637,642 and International Application No. PCT/US2019/056214 (Publication No. WO2020/117373), both of which are hereby incorporated in their entireties by reference.

### 5.5 Methods for a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient. Additional embodiments for such combination therapy have been described in U.S. Pat. No. 8,637,642 and International Application No. PCT/US2019/056214 (Publication No. WO2020/117373), both of which are hereby incorporated in their entireties by reference.

### 5.6 Doses for the Immune Checkpoint Inhibitors

In some embodiments, the amount of the checkpoint inhibitor for the various methods provided herein be determined by standard clinical techniques.

A dosage of the checkpoint inhibitor results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

### 5.7 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

In some embodiments, the ADC of the methods for which the various dosages are described in this Section (Section 5.7) is enfortumab vedotin (EV).

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses can be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses can be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein can be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment. In some embodiments, the ADC of the methods for which the various dosages are described in this paragraph is enfortumab vedotin (EV).

In some embodiments of the various methods provided herein, the ADC is administered at a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments, the ADC is administered at a dose of about 1 to about 10 mg/kg of the subject's body weight. In certain embodiments, the ADC is administered at a dose of about 1 to about 5 mg/kg of the subject's body weight. In other embodiments, the ADC is administered at a dose of about 1 to about 2.5 mg/kg of the subject's body weight. In further embodiments, the ADC is administered at a dose of about 1 to about 1.25 mg/kg of the subject's body weight. In some embodiments, the ADC is administered at a dose of about 0.25 mg/kg of the subject's body weight. In some embodiments, the ADC is administered at a dose of about 0.5 mg/kg of the subject's body weight. In some embodiments, the ADC is administered at a dose of about 0.75 mg/kg of the subject's body weight. In some embodiments, the ADC is administered at a dose of about 1.0 mg/kg of the subject's body weight. In some embodiments, the ADC is administered at a dose of about 1.25 mg/kg of the subject's body weight. In some embodiments, the ADC is administered at a dose of about 1.5 mg/kg of the subject's body weight. In some embodiments, the ADC is administered at a dose of about 1.75 mg/kg of the subject's body weight. In some embodiments, the ADC is administered at a dose of about 2.0 mg/kg of the subject's body weight. In some embodiments, the ADC is administered at a dose of about 2.25 mg/kg of the subject's body weight. In some embodiments, the ADC is administered at a dose of about 2.5 mg/kg of the subject's body weight.

In certain embodiments of the various methods provided herein, the ADC is administered at a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In some embodiments, the ADC is administered at a dose of 1 to 10 mg/kg of the subject's body weight. In certain embodiments, the ADC is administered at a dose of 1 to 5 mg/kg of the subject's body weight. In other embodiments, the ADC is administered at a dose of 1 to 2.5 mg/kg of the subject's body weight. In further embodiments, the ADC is administered at a dose of 1 to 1.25 mg/kg of the subject's body weight. In some embodiments, the ADC is administered at a dose of 0.25 mg/kg of the subject's body weight. In some embodiments, the ADC is administered at a dose of 0.5 mg/kg of the subject's body weight. In some embodiments, the ADC is administered at a dose of 0.75 mg/kg of the subject's body weight. In some embodiments, the ADC is administered at a dose of 1.0 mg/kg of the subject's body weight. In some embodiments, the ADC is administered at a dose of 1.25 mg/kg of the subject's body weight. In some embodiments, the ADC is administered at a dose of 1.5 mg/kg of the subject's body weight. In some embodiments, the ADC is administered at a dose of 1.75 mg/kg of the subject's body weight. In some embodiments, the ADC is administered at a dose of 2.0 mg/kg of the subject's body weight. In some embodiments, the ADC is administered at a dose of 2.25 mg/kg of the subject's body weight. In some embodiments, the ADC is administered at a dose of 2.5 mg/kg of the subject's body weight.

In some embodiments of the various methods provided herein, including those methods requiring a first and a second dose, the first dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the first dose of the ADC is a dose of about 1 to about 10 mg/kg of the subject's body weight. In some embodiments of the various methods provided herein, including those methods requiring a first and a second dose, the first dose of the ADC is a dose of about 1 to about 5 mg/kg of the subject's body weight. In some embodiments of the various methods provided herein, including those methods requiring a first and a second dose, the first dose of the ADC is a dose of about 1 to about 2.5 mg/kg of the subject's body weight. In some embodiments of the various methods provided herein, including those methods requiring a first and a second dose, the first dose of the ADC is a dose of about 1 to about 1.25 mg/kg of the subject's body weight. In some embodiments of the various methods provided herein, including those methods requiring a first and a second dose, the first dose of the ADC is a dose of about 0.25 mg/kg of the subject's body weight. In some embodiments of the various methods provided herein, including those methods requiring a first and a second dose, the first dose of the ADC is a dose of about 0.5 mg/kg of the subject's body weight. In some embodiments, the first dose of the ADC is a dose of about 0.75 mg/kg of the subject's body weight. In some embodiments, the first dose of the ADC is a dose of about 1.0 mg/kg of the subject's body weight. In some embodiments, the first dose of the ADC is a dose of about 1.25 mg/kg of the subject's body weight. In some embodiments, the first dose of the ADC is a dose of about 1.5 mg/kg of the subject's body weight. In some embodiments, the first dose of the ADC is a dose of about 1.75 mg/kg of the subject's body weight. In some embodiments, the first dose of the ADC is a dose of about 2.0 mg/kg of the subject's body weight. In some embodiments, the first dose of the ADC is a dose of about 2.25 mg/kg of the subject's body weight. In some embodiments, the first dose of the ADC is a dose of or about 2.5 mg/kg of the subject's body weight.

In certain embodiments of the various methods provided herein, including those methods requiring a first and a second dose, the first dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the first dose of the ADC is a dose of 1 to 10 mg/kg of the subject's body weight. In some embodiments of the various methods provided herein, including those methods requiring a first and a second dose, the first dose of the ADC is a dose of 1 to 5 mg/kg of the subject's body weight. In some embodiments of the various methods provided herein, including those methods requiring a first and a second dose, the first dose of the ADC is a dose of 1 to 2.5 mg/kg of the subject's body weight. In some embodiments of the various methods provided herein, including those methods requiring a first and a second dose, the first dose of the ADC is a dose of 1 to 1.25 mg/kg of the subject's body weight. In some embodiments of the various methods provided herein, including those methods requiring a first and a second dose, the first dose of the ADC is a dose of 0.25 mg/kg of the subject's body weight. In some embodiments of the various methods provided herein, including those methods requiring a first and a second dose, the first dose of the ADC is a dose of 0.5 mg/kg of the subject's body weight. In some embodiments, the first dose of the ADC is a dose of 0.75 mg/kg of the subject's body weight. In some embodiments, the first dose of the ADC is a dose of 1.0 mg/kg of the subject's body weight. In some embodiments, the first dose of the ADC is a dose of 1.25 mg/kg of the subject's body weight. In some embodiments, the first dose of the ADC is a dose of 1.5 mg/kg of the subject's body weight. In some embodiments, the first dose of the ADC is a dose of 1.75 mg/kg of the subject's body weight. In some embodiments, the first dose of the ADC is a dose of 2.0 mg/kg of the subject's body weight. In some embodiments, the first dose of the ADC is a dose of 2.25 mg/kg of the subject's body weight. In some embodiments, the first dose of the ADC is a dose of 2.5 mg/kg of the subject's body weight.

In some embodiments of the various methods provided herein, including those methods requiring a first and a second dose, the second dose of the ADC is lower than the first dose by about 0.1 mg/kg to about 2 mg/kg of the subject's body weight. In some embodiments of the various methods provided herein, including those methods requiring a first and a second dose, the second dose of the ADC is lower than the first dose by about 0.1 mg/kg of the subject's body weight. In some embodiments of the various methods provided herein, including those methods requiring a first and a second dose, the second dose of the ADC is lower than the first dose by about 0.2 mg/kg of the subject's body weight. In some embodiments of the various methods provided herein, including those methods requiring a first and a second dose, the second dose of the ADC is lower than the first dose by about 0.25 mg/kg of the subject's body weight. In some embodiments of the various methods provided herein, including those methods requiring a first and a second dose, the second dose of the ADC is lower than the first dose by about 0.3 mg/kg of the subject's body weight. In some embodiments of the various methods provided herein, including those methods requiring a first and a second dose, the second dose of the ADC is lower than the first dose by about 0.4 mg/kg of the subject's body weight. In some embodiments of the various methods provided herein, including those methods requiring a first and a second dose, the second dose of the ADC is lower than the first dose by about 0.5 mg/kg of the subject's body weight. In some embodiments of the various methods provided herein, including those methods requiring a first and a second dose, the second dose of the ADC is lower than the first dose by about 0.6 mg/kg of the subject's body weight. In some embodiments of the various methods provided herein, including those methods requiring a first and a second dose, the second dose of the ADC is lower than the first dose by about 0.7 mg/kg of the subject's body weight. In some embodiments of the various methods provided herein, including those methods requiring a first and a second dose, the second dose of the ADC is lower than the first dose by about 0.75 mg/kg of the subject's body weight. In some embodiments of the various methods provided herein, including those methods requiring a first and a second dose, the second dose of the ADC is lower than the first dose by about 0.8 mg/kg of the subject's body weight. In some embodiments of the various methods provided herein, including those methods requiring a first and a second dose, the second dose of the ADC is lower than the first dose by about 0.9 mg/kg of the subject's body weight. In some embodiments of the various methods provided herein, including those methods requiring a first and a second dose, the second dose of the ADC is lower than the first dose by about 1 mg/kg of the subject's body weight. In some embodiments of the various methods provided herein, including those methods requiring a first and a second dose, the second dose of the ADC is lower than the first dose by about 1.1 mg/kg of the subject's body weight. In some embodiments of the various methods provided herein, including those methods requiring a first and a second dose, the second dose of the ADC is lower than the first dose by about 1.2 mg/kg of the subject's body weight. In some embodiments of the various methods provided herein, including those methods requiring a first and a second dose, the second dose of the ADC is lower than the first dose by about 1.25 mg/kg of the subject's body weight. In some embodiments of the various methods provided herein, including those methods requiring a first and a second dose, the second dose of the ADC is lower than the first dose by about 1.3 mg/kg of the subject's body weight. In some embodiments of the various methods provided herein, including those methods requiring a first and a second dose, the second dose of the ADC is lower than the first dose by about 1.4 mg/kg of the subject's body weight. In some embodiments of the various methods provided herein, including those methods requiring a first and a second dose, the second dose of the ADC is lower than the first dose by about 1.5 mg/kg of the subject's body weight. In some embodiments of the various methods provided herein, including those methods requiring a first and a second dose, the second dose of the ADC is lower than the first dose by about 1.6 mg/kg of the subject's body weight. In some embodiments of the various methods provided herein, including those methods requiring a first and a second dose, the second dose of the ADC is lower than the first dose by about 1.7 mg/kg of the subject's body weight. In some embodiments of the various methods provided herein, including those methods requiring a first and a second dose, the second dose of the ADC is lower than the first dose by about 1.75 mg/kg of the subject's body weight. In some embodiments of the various methods provided herein, including those methods requiring a first and a second dose, the second dose of the ADC is lower than the first dose by about 1.8 mg/kg of the subject's body weight. In some embodiments of the various methods provided herein, including those methods requiring a first and a second dose, the second dose of the ADC is lower than the first dose by about 1.9 mg/kg of the subject's body weight. In some embodiments of the various methods provided herein, including those methods requiring a first and a second dose, the second dose of the ADC is lower than the first dose by about 2 mg/kg of the subject's body weight.

In certain embodiments of the various methods provided herein, including those methods requiring a first and a second dose, the second dose of the ADC is lower than the first dose by 0.1 mg/kg to 2 mg/kg of the subject's body weight. In some embodiments of the various methods provided herein, including those methods requiring a first and a second dose, the second dose of the ADC is lower than the first dose by 0.1 mg/kg of the subject's body weight. In some embodiments of the various methods provided herein, including those methods requiring a first and a second dose, the second dose of the ADC is lower than the first dose by 0.2 mg/kg of the subject's body weight. In some embodiments of the various methods provided herein, including those methods requiring a first and a second dose, the second dose of the ADC is lower than the first dose by 0.25 mg/kg of the subject's body weight. In some embodiments of the various methods provided herein, including those methods requiring a first and a second dose, the second dose of the ADC is lower than the first dose by 0.3 mg/kg of the subject's body weight. In some embodiments of the various methods provided herein, including those methods requiring a first and a second dose, the second dose of the ADC is lower than the first dose by 0.4 mg/kg of the subject's body weight. In some embodiments of the various methods provided herein, including those methods requiring a first and a second dose, the second dose of the ADC is lower than the first dose by 0.5 mg/kg of the subject's body weight. In some embodiments of the various methods provided herein, including those methods requiring a first and a second dose, the second dose of the ADC is lower than the first dose by 0.6 mg/kg of the subject's body weight. In some embodiments of the various methods provided herein, including those methods requiring a first and a second dose, the second dose of the ADC is lower than the first dose by 0.7 mg/kg of the subject's body weight. In some embodiments of the various methods provided herein, including those methods requiring a first and a second dose, the second dose of the ADC is lower than the first dose by 0.75 mg/kg of the subject's body weight. In some embodiments of the various methods provided herein, including those methods requiring a first and a second dose, the second dose of the ADC is lower than the first dose by 0.8 mg/kg of the subject's body weight. In some embodiments of the various methods provided herein, including those methods requiring a first and a second dose, the second dose of the ADC is lower than the first dose by 0.9 mg/kg of the subject's body weight. In some embodiments of the various methods provided herein, including those methods requiring a first and a second dose, the second dose of the ADC is lower than the first dose by 1 mg/kg of the subject's body weight. In some embodiments of the various methods provided herein, including those methods requiring a first and a second dose, the second dose of the ADC is lower than the first dose by 1.1 mg/kg of the subject's body weight. In some embodiments of the various methods provided herein, including those methods requiring a first and a second dose, the second dose of the ADC is lower than the first dose by 1.2 mg/kg of the subject's body weight. In some embodiments of the various methods provided herein, including those methods requiring a first and a second dose, the second dose of the ADC is lower than the first dose by 1.25 mg/kg of the subject's body weight. In some embodiments of the various methods provided herein, including those methods requiring a first and a second dose, the second dose of the ADC is lower than the first dose by 1.3 mg/kg of the subject's body weight. In some embodiments of the various methods provided herein, including those methods requiring a first and a second dose, the second dose of the ADC is lower than the first dose by 1.4 mg/kg of the subject's body weight. In some embodiments of the various methods provided herein, including those methods requiring a first and a second dose, the second dose of the ADC is lower than the first dose by 1.5 mg/kg of the subject's body weight. In some embodiments of the various methods provided herein, including those methods requiring a first and a second dose, the second dose of the ADC is lower than the first dose by 1.6 mg/kg of the subject's body weight. In some embodiments of the various methods provided herein, including those methods requiring a first and a second dose, the second dose of the ADC is lower than the first dose by 1.7 mg/kg of the subject's body weight. In some embodiments of the various methods provided herein, including those methods requiring a first and a second dose, the second dose of the ADC is lower than the first dose by 1.75 mg/kg of the subject's body weight. In some embodiments of the various methods provided herein, including those methods requiring a first and a second dose, the second dose of the ADC is lower than the first dose by 1.8 mg/kg of the subject's body weight. In some embodiments of the various methods provided herein, including those methods requiring a first and a second dose, the second dose of the ADC is lower than the first dose by 1.9 mg/kg of the subject's body weight. In some embodiments of the various methods provided herein, including those methods requiring a first and a second dose, the second dose of the ADC is lower than the first dose by 2 mg/kg of the subject's body weight.

In some embodiments of the various methods provided herein, including those methods requiring a first and a second dose, the second dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the second dose of the ADC is a dose of about 1 to about 10 mg/kg of the subject's body weight. In some embodiments of the various methods provided herein, including those methods requiring a first and a second dose, the second dose of the ADC is a dose of about 1 to about 5 mg/kg of the subject's body weight. In some embodiments of the various methods provided herein, including those methods requiring a first and a second dose, the second dose of the ADC is a dose of about 1 to about 2.5 mg/kg of the subject's body weight. In some embodiments of the various methods provided herein, including those methods requiring a first and a second dose, the second dose of the ADC is a dose of about 1 to about 1.25 mg/kg of the subject's body weight. In some embodiments of the various methods provided herein, including those methods requiring a first and a second dose, the second dose of the ADC is a dose of about 0.25 mg/kg of the subject's body weight. In some embodiments of the various methods provided herein, including those methods requiring a first and a second dose, the second dose of the ADC is a dose of about 0.5 mg/kg of the subject's body weight. In some embodiments of the various methods provided herein, including those methods requiring a first and a second dose, the second dose of the ADC is a dose of about 0.75 mg/kg of the subject's body weight. In some embodiments of the various methods provided herein, including those methods requiring a first and a second dose, the second dose of the ADC is a dose of about 1.0 mg/kg of the subject's body weight. In some embodiments of the various methods provided herein, including those methods requiring a first and a second dose, the second dose of the ADC is a dose of about 1.25 mg/kg of the subject's body weight. In some embodiments of the various methods provided herein, including those methods requiring a first and a second dose, the second dose of the ADC is a dose of about 1.5 mg/kg of the subject's body weight. In some embodiments of the various methods provided herein, including those methods requiring a first and a second dose, the second dose of the ADC is a dose of about 1.75 mg/kg of the subject's body weight. In some embodiments of the various methods provided herein, including those methods requiring a first and a second dose, the second dose of the ADC is a dose of about 2.0 mg/kg of the subject's body weight. In some embodiments of the various methods provided herein, including those methods requiring a first and a second dose, the second dose of the ADC is a dose of about 2.25 mg/kg of the subject's body weight. In some embodiments of the various methods provided herein, including those methods requiring a first and a second dose, the second dose of the ADC is a dose of about 2.5 mg/kg of the subject's body weight.

In certain embodiments of the various methods provided herein, including those methods requiring a first and a second dose, the second dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the second dose of the ADC is a dose of 1 to 10 mg/kg of the subject's body weight. In some embodiments of the various methods provided herein, including those methods requiring a first and a second dose, the second dose of the ADC is a dose of 1 to 5 mg/kg of the subject's body weight. In some embodiments of the various methods provided herein, including those methods requiring a first and a second dose, the second dose of the ADC is a dose of 1 to 2.5 mg/kg of the subject's body weight. In some embodiments of the various methods provided herein, including those methods requiring a first and a second dose, the second dose of the ADC is a dose of 1 to 1.25 mg/kg of the subject's body weight. In some embodiments of the various methods provided herein, including those methods requiring a first and a second dose, the second dose of the ADC is a dose of 0.25 mg/kg of the subject's body weight. In some embodiments of the various methods provided herein, including those methods requiring a first and a second dose, the second dose of the ADC is a dose of 0.5 mg/kg of the subject's body weight. In some embodiments of the various methods provided herein, including those methods requiring a first and a second dose, the second dose of the ADC is a dose of 0.75 mg/kg of the subject's body weight. In some embodiments of the various methods provided herein, including those methods requiring a first and a second dose, the second dose of the ADC is a dose of 1.0 mg/kg of the subject's body weight. In some embodiments of the various methods provided herein, including those methods requiring a first and a second dose, the second dose of the ADC is a dose of 1.25 mg/kg of the subject's body weight. In some embodiments of the various methods provided herein, including those methods requiring a first and a second dose, the second dose of the ADC is a dose of 1.5 mg/kg of the subject's body weight. In some embodiments of the various methods provided herein, including those methods requiring a first and a second dose, the second dose of the ADC is a dose of 1.75 mg/kg of the subject's body weight. In some embodiments of the various methods provided herein, including those methods requiring a first and a second dose, the second dose of the ADC is a dose of 2.0 mg/kg of the subject's body weight. In some embodiments of the various methods provided herein, including those methods requiring a first and a second dose, the second dose of the ADC is a dose of 2.25 mg/kg of the subject's body weight. In some embodiments of the various methods provided herein, including those methods requiring a first and a second dose, the second dose of the ADC is a dose of 2.5 mg/kg of the subject's body weight.

In certain embodiments of the various methods provided herein, including those methods requiring a first and a second dose, the second dose of the ADC is identical to the first dose of the ADC.

In some embodiments of the methods provided herein, the ADC is administered by an intravenous (IV) injection or infusion. In one embodiment, the first dose of the ADC is administered by an IV injection. In another embodiment, the first dose of the ADC is administered by an IV infusion. In yet another embodiment, the second dose of the ADC is administered by an IV injection. In yet another embodiment, the second dose of the ADC is administered by an IV injection infusion. In one embodiment, the first dose of the ADC is administered by an IV injection and the second dose of the ADC is administered by an IV injection. In another embodiment, the first dose of the ADC is administered by an IV infusion and the second dose of the ADC is administered by an IV injection. In yet another embodiment, the second dose of the ADC is administered by an IV injection and the second dose of the ADC is administered by an IV injection infusion. In yet another embodiment, the second dose of the ADC is administered by an IV injection infusion and the second dose of the ADC is administered by an IV injection infusion. In some embodiments, the ADC of the methods for which the various dosages are described in this paragraph is enfortumab vedotin (EV).

In certain embodiments of the methods provided herein, the ADC is administered by an IV injection or infusion three times every four-week cycle. In some embodiments of the methods provided herein, the first dose of the ADC is administered by an IV injection or infusion three times every four-week cycle. In some embodiments of the methods provided herein, the second dose of the ADC is administered by an IV injection or infusion three times every four-week cycle. In some embodiments of the methods provided herein, the first dose of the ADC is administered by an IV injection or infusion three times every four-week cycle and the second dose of the ADC is administered by an IV injection or infusion three times every four-week cycle. In some embodiments, the ADC of the methods for which the various dosages are described in this paragraph is enfortumab vedotin (EV).

In some embodiments of the methods provided herein, the ADC is administered by an IV injection or infusion on Days 1, 8 and 15 of every four-week cycle. In some embodiments, the first dose of ADC is administered by an IV injection or infusion on Days 1, 8 and 15 of every four-week cycle. In some embodiments, the second dose of ADC is administered by an IV injection or infusion on Days 1, 8 and 15 of every four-week cycle. In some embodiments, the first dose of ADC is administered by an IV injection or infusion on Days 1, 8 and 15 of every four-week cycle and the second dose of ADC is administered by an IV injection or infusion on Days 1, 8 and 15 of every four-week cycle. In some embodiments, the ADC of the methods for which the various dosages are described in this paragraph is enfortumab vedotin (EV).

In certain embodiments of the methods provided herein, the ADC is administered by an IV injection or infusion over about 30 minutes three times every four-week cycle. In some embodiments, the first dose of the ADC is administered by an IV injection or infusion over about 30 minutes three times every four-week cycle. In some embodiments, the second dose of the ADC is administered by an IV injection or infusion over about 30 minutes three times every four-week cycle. In some embodiments, the first dose of the ADC is administered by an IV injection or infusion over about 30 minutes three times every four-week cycle and the second dose of the ADC is administered by an IV injection or infusion over about 30 minutes three times every four-week cycle. In some embodiments, the ADC of the methods for which the various dosages are described in this paragraph is enfortumab vedotin (EV).

In some embodiments of the methods provided herein, the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle. In some embodiments of the methods provided herein, the second dose of the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle and the second dose of the ADC is administered by an IV injection or infusion over about 30 minutes on Days 1, 8 and 15 of every four-week cycle. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 1 mg/kg, 1.25 mg/kg, or about 1.5 mg/kg of the subject's body weight by an intravenous (IV) injection or infusion over about 30 minutes three times every 28-day cycle. In some embodiments, the antibody drug conjugate formulated in the pharmaceutical composition is administered by an intravenous (IV) injection or infusion over about 30 minutes on Days 1, 8 and 15 of every 28-day cycle. In some embodiments, the method further comprises administering an immune checkpoint inhibitor by an intravenous (IV) injection or infusion one or more times in each four-week cycle. In some embodiments of the methods provided herein, the ADC is administered three times within a 28 day cycle. In some embodiments of the methods provided herein, the ADC is administered on Days 1, 8 and 15 of a 28 day cycle. In some embodiments, the ADC of the methods for which the various dosages are described in this paragraph is enfortumab vedotin (EV).

In one specific embodiment of the methods provided herein, the ADC has the following structure:

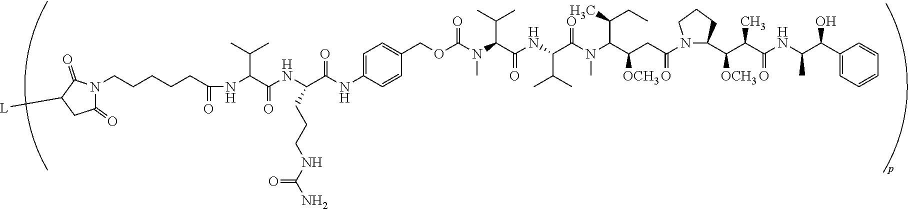

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on Days 1, 8 and 15 of every four-week cycle.

### 5.8 Methods for Determining the Biomarkers

The disclosure provides that the expression of any of the markers provided herein can be determined by various methods known in the field. In some embodiments, the expression of the markers can be determined by the amount or relative amount of mRNA transcribed from the marker genes. In one embodiment, the expression of the marker genes can be determined by the amount or relative amount of the protein products encoded by the marker genes. In another embodiment, the expression of the marker genes can be determined by the level of biological or chemical response induced by the protein products encoded by the marker genes. Additionally, in certain embodiments, the expression of the marker genes can be determined by the expression of one or more genes that correlates with the expression of the marker genes.

As described above, levels or amounts of gene transcripts (e.g. mRNA) of the marker genes can be used as a proxy for the expression levels of markers genes. Numerous different PCR or qPCR protocols are known in the art including those exemplified herein. In some embodiments, the various PCR or qPCR methods are applied or adapted for determining the mRNA level of the various marker genes. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005): D'haene et al., Methods 50:262-270 (2010)), which is incorporated by reference herein in its entirety. Examples of PCR assays can also be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the mRNA levels of the marker genes as follows. Briefly, mean Ct (cycle threshold) values (or referred to herein interchangeably as Cq (quantification cycle)) of replicate qPCR reactions for the marker genes and one or more housekeeping genes are determined. Mean Ct values for the marker genes can be then normalized to the Ct values of the housekeeping genes using the following exemplary formula: marker-gene-ΔCt=(mean Ct of marker gene-mean Ct of housekeeping gene A). The relative marker-gene-ΔCt can then be used to determine relative level of marker gene mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art can also be used for the quantification of RNA transcripts of the marker genes in a sample as the proxy for the expression of the marker genes, including northern blotting and in situ hybridization (Parker & Barnes, Methods in Molecular Biology 106:247-283 (1999)); RNAse protection assays (Hod, Biotechniques 13:852-854 (1992)), microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. ISH is a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization. BIOS Scientific Publishers Ltd. Oxford (2000)). RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6);el325. doi: 10.7759/cureus.1325 (2017); Weier et al., Expert Rev. Mol. Diagn. 2(2):109-119 (2002)). Fluorescent RNA ISH utilizes fluorescent dyes and fluorescent microscopes for RNA labeling and detection, respectively. Fluorescent RNA ISH can provides for multiplexing of four to five target sequences.

Alternatively, RNA transcripts of the marker genes in a sample as the proxy for the expression of the marker genes can be determined by sequencing techniques. Representative methods for sequencing-based gene expression analysis include Serial Analysis of Gene Expression (SAGE), and gene expression analysis by massively parallel signature sequencing (MPSS).

In some embodiments, expression of the marker genes can be determined by the relative abundance of the RNA transcripts (including for example mRNA) of the marker genes in a pool of total transcribed RNA. Such relative abundance of the RNA transcripts of the marker genes can be determined by next generation sequencing, which is known as RNA-seq. In one example of the RNA-seq procedure, RNAs from different sources (blood, tissue, cells) are purified, optionally enriched (e.g. with oligo (dT) primers), converted to cDNA, and fragmented. Millions or even billions of short sequence reads are generated from the randomly fragmented cDNA library. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/ma.074922.120, all of which are incorporated herein in their entirety by reference. The expression level of each mRNA transcript of the marker genes is determined by the total number of mapped fragments upon normalization, which is directly proportional to its abundance level. A few normalization schemes are known and used to facilitate the use of the abundance of the RNA transcripts as the parameter for determining gene expression, including RPKM (Reads Per Kilobase Million), FPKM (Fragments Per Kilobase Million), and/or TPM (Transcripts Per Kilobase Million). Briefly, RPKM can be calculated as follows: count up the total reads in a sample and divide that number by 1,000,000—which is the “per million” scaling factor; divide the read counts by the “per million” scaling factor, which normalizes for sequencing depth, giving the reads per million (RPM); and divide the RPM values by the length of the gene, in kilobases, which gives RPKM. FPKM is closely related to RPKM except with fragment replacing read. RPKM was made for single-end RNA-seq, where every read corresponded to a single fragment that was sequenced. FPKM was made for paired-end RNA-seq, in which two reads can correspond to a single fragment, or, if one read in the pair did not map, one read can correspond to a single fragment. TPM is very similar to RPKM and FPKM and is calculated as follows: divide the read counts by the length of each gene in kilobases, which gives the reads per kilobase (RPK); count up all the RPK values in a sample and divide this number by 1,000,000, which gives the “per million” scaling factor; divide the RPK values by the “per million” scaling factor, which gives TPM. See Zhao et al. BMC genomics 16: 97 (2015); Zhao et al. Scientific Reports 8: 4781 (2018), Shanrong Zhao et al., RNA, published in advance Apr. 13, 2020, doi: 10.1261/ma.074922.120, all of which are incorporated herein in their entirety by reference.

In one embodiment, the expression of the marker genes is determined by RNA-seq, for example by TPM, RPKM, and/or FPKM. In some embodiments, the expression of the marker genes is determined by TPM. In some embodiments, the expression of the marker genes is determined by RPKM. In some embodiments, the expression of the marker genes is determined by FPKM.

As described earlier, the expression of the marker genes can be determined in a sample from a subject. In some embodiments, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a tissue sample. In some embodiments, the tissue sample is tissue fractions isolated or extracted from a mammal, in particular a human. In some embodiments, the tissue sample is a population of cells isolated or extracted from a mammal, in particular a human. In some embodiments, the tissue sample is a sample obtained from a biopsy. In certain embodiments, the samples can be obtained from a variety of organs of a subject, including a human subject. In some embodiments, the samples are obtained from organs of a subject having a cancer. In some embodiments, the samples are obtained from organs having a cancer in a subject having a cancer. In other embodiments, the samples, for example reference samples, are obtained from normal organs from the patient or from a second human subject.

In certain embodiments of the methods provided herein, the tissue includes a tissue from bladder, ureter, breast, lung, colon, rectum, ovary, Fallopian tube, esophagus, cervix, uterine endometrium, skin, larynx, bone marrow, salivary gland, kidney, prostate, brain, spinal cord, placenta, adrenal, pancreas, parathyroid, hypophysis, testis, thyroid, spleen, tonsil, thymus, heart, stomach, small intestine, liver, skeletal muscle, peripheral nerve, mesothelium, or eye.

In further embodiments of the methods provided herein, the expression of the various marker genes can be detected by a variety of immunoassays known in the art, including an immunohistochemistry (IHC) assay, an immunoblotting assay, a FACS assay, and an ELISA.

The expression of the various marker genes can be detected by antibodies against the protein products encoded by the marker genes in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein products encoded by the marker genes, can be used to detect expression of the marker genes in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of the protein products encoded by the marker genes in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3,3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system. HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of the marker genes is detected with IHC using a signal amplification system. In some embodiments, the specimen is then counterstained to identify cellular and subcellular elements.

In some embodiments, the expression level of the protein products encoded by the marker genes can also be detected with antibodies against the protein products encoded by the marker genes using an immunoblotting assay. In some embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein products encoded by the marker genes, can be used to detect expression of the marker genes. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of the protein products encoded by the marker genes can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein products encoded by the marker genes, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the expression level of the target protein. Therefore, the expression level of the protein products encoded by the marker genes can be detected using antibodies against such protein products. Non-fluorescent cytoplasmic proteins can also be observed by staining permeabalized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of the protein products encoded by the marker genes can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assay format. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the ETA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase,6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present disclosure.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques. Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including κ-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof, CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

The methods provided in this Section (Section 5.8) can be used with various cancer models known in the art. In one embodiment, mouse xenograft cancer models are used. Briefly, T-24 and UM-UC-3 cells are purchased from ATCC and cultured using the recommended media conditions. The T-24 hNectin-4 (human nectin-4) and the UM-UC-3 Nectin-4 cells are generated by transducing parental cells with lentivirus containing the human Nectin-4 using the pRCDCMEP-CMV-hNectin-4 EF1-Puro construct and selected using puromycin. The T-24 Nectin-4 (clone 1A9) cells are implanted into nude mice and passaged via trocar, allowed to reach approximately 200 mm³ tumor volume, and subsequently treated with a single intraperitoneal (IP) dose of enfortumab vedotin (3 mg/kg) or non-binding ADC (3 mg/kg) with 7 animals per treatment group. Follow-up ICD studies with this model involve collecting tumors 5 days post treatment for downstream analysis by RNA-seq, flow, immunohistochemistry (IHC), and Luminex. Tumors are fixed in formalin and prepared as FFPE tissue blocks. Blocks are cut at 4 μm and immunohistochemistry is performed using F4/80, CD11c. The immunohistochemically stained slides sections are scanned with a Leica AT2 digital whole slide scanner, and the images are analyzed with Visiopharm software by use of custom-made algorithms for Nectin 4, CD11c and F4/80 staining. The algorithms are optimized on the basis of staining intensity and background staining. Percent positive staining is calculated for Nectin 4 and positive cells per mm² is calculated for F480 and CD11c.

Sections of tumor are lysed in Cell Lysis Buffer 2 (R&D Systems®, Catalog #895347). The cytokines and chemokines from the tumor samples are measured using the MILLIPLEX MAP mouse cytokine/chemokine magnetic bead panel (Millipore) and read on the LUMINEX MAGPIX system.

For the RNA-seq analysis RNA from flash frozen tumors is isolated using the TRIZOL Plus RNA Purification Kit (Life Technologies) according to the manufacturer's protocol yielding high quality RNA (average RNA integrity number >8). RNA selection method is using Poly(A) selection and the mRNA Library Prep Kit from Illumina and read on the Hi-Seq 2×150 bp, single index (Illumina). The sequence reads are mapped to the human and mouse transcriptome and total reads per million were determined.

The disclosure is generally provided using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

Particular embodiments of this disclosure are described herein, including the best mode known to the inventors for carrying out the disclosure. Upon reading the foregoing description, variations of the disclosed embodiments can become apparent to individuals working in the art, and it is expected that those skilled artisans can employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference in their entireties as if each individual publication or patent application were specifically and individually indicated to be incorporated by reference.

A number of embodiments of the disclosure have been described. Nevertheless, it will be understood that various modifications can be made without departing from the spirit and scope of the disclosure.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1—A Single-Arm, Open-Label, Multicenter Study of Enfortumab Vedotin (ASG-22CE) for Treatment of Patients with Locally Advanced or Metastatic Urothelial Cancer Who Previously Received Immune Checkpoint Inhibitor (CPI) Therapy

### 6.1.1 Drug Used in the Clinical Study

Enfortumab vedotin is a Nectin-4 targeted monoclonal antibody (AGS-22C3) covalently linked to the microtubule-disrupting agent monomethyl auristatin E (MMAE). Enfortumab vedotin consists of three functional subunits:
A fully human IgG1K antibody (AGS-22C3);
The microtubule-disrupting agent MMAE;
A protease-cleavable maleimidocaproyl-valine-citrulline (vc) linker that covalently attaches MMAE to AGS-22C3.

Enfortumab vedotin binds the V domain of Nectin-4 (Challita-Eid et al., Cancer Res (2016); 76(10): 3003-13.). In the presumed mechanism of action, the drug binds Nectin-4 protein on the cell surface and is internalized, causing proteolytic cleavage of the vc linker and intracellular release of MMAE. Free MMAE subsequently disrupts tubulin polymerization and leads to mitotic arrest.

### 6.1.2 Summary of the Study

6.1.2.1 Synopsis

(i) Name of Study Drug

Enfortumab Vedotin (ASG-22CE)

(ii) Phase of Development

Phase 2

(iii) Title of Study

A single-arm, open-label, multicenter study of enfortumab vedotin (ASG-22CE) for treatment of patients with locally advanced or metastatic urothelial cancer who previously received immune checkpoint inhibitor (CPI) therapy

(iv) Study Objective(s)

Primary
To determine the antitumor activity of single-agent enfortumab vedotin as measured by confirmed objective response rate (ORR) in patients with locally advanced or metastatic urothelial cancer who have previously received systemic therapy with a CPI and either previously received platinum-containing chemotherapy or are platinum-naïve and cisplatin-ineligible

Secondary
To assess duration of response (DOR)
To assess disease control rate (DCR)
To assess progression-free survival (PFS)
To assess overall survival (OS)
To assess the safety and tolerability of enfortumab vedotin
To assess the pharmacokinetics (PK) of enfortumab vedotin
To assess the incidence of antitherapeutic antibodies (ATA)

Additional
To explore potential correlations between biomarkers and clinical outcomes
To evaluate the treatment effect of enfortumab vedotin on quality of life (QoL)

(v) Study Population

The population to be studied includes patients with locally advanced or metastatic urothelial cancer who previously received therapy with CPI, with measurable disease according to Response Evaluation Criteria in Solid Tumors (RECIST) Version 1.1. Patients who received CPI therapy in the neoadjuvant/adjuvant setting and had recurrent or progressive disease (PD) either during therapy or within 3 months of therapy completion are eligible.

Patients must also be either: 1) Platinum-treated (Cohort 1): Patients who received prior treatment with platinum-containing chemotherapy defined as those who received platinum in the adjuvant/neoadjuvant setting and had recurrent or progressive disease within 12 months of completion OR received treatment with platinum in the locally advanced (defined as unresectable with curative intent) or metastatic setting; OR 2) Platinum-naïve and cisplatin ineligible (Cohort 2): Patients who have not received prior platinum-containing or other chemotherapy in the locally advanced or metastatic setting and are ineligible for treatment with cisplatin at time of enrollment. Patients who received platinum in the adjuvant/neoadjuvant setting and did not progress within 12 months of completion will be considered platinum-naïve.

Patients must have PD during or following their most recent therapy. Eligible patients must be ≥18 years of age, and legally an adult according to local regulation. Patients must have an Eastern Cooperative Oncology Group (ECOG) performance status score of ≤1 for Cohort 1, or ECOG≤2 for Cohort 2. Patients must have an anticipated life expectancy of ≥3 months as assessed by the investigator. Patients must have adequate baseline hematologic, hepatic, and renal function.

Patients must not have ongoing sensory or motor neuropathy (Grade 2 or higher), or active central nervous system metastases. Patients must not have been previously enrolled in an enfortumab vedotin study or previously treated with other monomethyl auristatin E (MMAE)-based antibody-drug conjugates (ADCs). There are no limits for prior lines of therapy, including taxanes. Patients must not have a history of another malignancy within 3 years, or any evidence of residual disease from a previously diagnosed malignancy.

Patients are also excluded if they are currently receiving systemic antimicrobial treatment for active infection or high dose steroids. Patients with uncontrolled diabetes are excluded. Uncontrolled diabetes is defined as hemoglobin A1C (HbA1c)≥8% or HbA1c 7-<8% with associated diabetes symptoms (polyuria or polydipsia) that are not otherwise explained. Patients must not have uncontrolled tumor-related bone pain or impending spinal cord compression. Patients requiring pain medication must be on a stable regimen at the time of enrollment (a minimum of 2 weeks).

(vi) Number of Planned Patients

Approximately 200 patients will be enrolled in this study, including approximately 100 or more platinum-treated patients (Cohort 1), and up to approximately 100 platinum-naïve and cisplatin-ineligible patients (Cohort 2).

(vii) Study Design

This is a single-arm, open-label, multicenter trial designed to assess the efficacy and safety of enfortumab vedotin as a single agent in locally advanced or metastatic urothelial cancer patients who have previously received systemic therapy with a CPI. For the purpose of this study a CPI is defined as a programmed cell death protein 1 (PD-1) inhibitor or programmed death-ligand 1 (PD-L1) inhibitor (including, but not limited to: atezolizumab, pembrolizumab, durvalumab, avelumab, and nivolumab). Patients must either have received prior treatment with platinum-containing chemotherapy (Cohort 1) or received no prior treatment with platinum-containing or other chemotherapy and are ineligible for treatment with cisplatin at time of enrollment (Cohort 2).

Enfortumab vedotin at a dose of 1.25 mg/kg will be administered as an intravenous (IV) infusion over approximately 30 minutes on Days 1, 8, and 15 of each 28-day cycle. Patients will continue to receive study treatment until disease progression, unacceptable toxicity, investigator decision, consent withdrawal, start of a subsequent anticancer therapy, pregnancy, or study termination by the sponsor. After discontinuation of study treatment, patients will be followed every 8 weeks (1 week) for response assessments, ECOG performance status, and physical exams. After 1 year of on study, the frequency of follow-up exams including response assessments will be reduced to every 12 weeks (+1 week). Patients that have progressed or begun subsequent anticancer therapy will be contacted every 8 weeks (1 week) up to 1 year on study, and every 12 weeks (+1 week) thereafter to obtain information on subsequent anticancer therapy, and survival status until death, study closure, or withdrawal of consent, whichever occurs first. The study will be closed 5 years after enrollment of the last patient, or when no patients remain in long-term follow-up, whichever occurs first. Additionally, the sponsor may terminate the study at any time.

On a periodic basis, an independent data monitoring committee (IDMC) will monitor the safety of patients participating in this trial. The IDMC will be responsible for evaluating the results of safety analyses and will make recommendations to the sponsor. An ongoing real-time review of patient safety and serious adverse events (SAEs) will also be conducted by the sponsor's Drug Safety Department.

(viii) Test Product, Dose, and Mode of Administration

Enfortumab vedotin 1.25 mg/kg will be administered as an IV infusion over approximately 30 minutes on Day 1, 8, and 15 of each 28-day cycle

(ix) Duration of Treatment

Patients may continue on study treatment until disease progression, unacceptable toxicity, investigator decision, consent withdrawal, start of a subsequent anticancer therapy, pregnancy, or study termination by the sponsor.

(x) Efficacy Assessments

Measures of anticancer activity will be assessed by computed tomography (CT) scans with contrast approximately every 8 weeks (±1 week). After 1 year on study, response assessments will be reduced to every 12 weeks (±1 week). The schedule of response assessments should not be adjusted for dose delays/interruptions or other reasons for changes in the timing of a patient's study activities; timepoints for response assessments should be calculated from Cycle 1 Day 1 during treatment. For patients who cannot receive CT scans with contrast, other protocol-specified imaging methods may be used. Patients must be evaluated using the same imaging method throughout the study for efficacy assessments. CT scans with contrast at the minimum will include the chest, abdomen, and pelvis. Other regions should be scanned if the patient has known or suspected disease in that region. Responses (complete response (CR) or partial response (PR)) will be confirmed with repeat scans 4 weeks (+1 week window) after first documentation of response. The determination of antitumor activity will be based on confirmed objective response assessments as defined by RECIST Version 1.1.

Response and progression will be assessed by an independent review facility (IRF). The investigator will make treatment decisions based on site assessments of scans by RECIST.

Patients who discontinue study treatment for reasons other than objective disease progression by RECIST will continue to receive scans 8 weeks (±1 week) after the previous response assessment scan and every 8 weeks (±1 week) following the previous scan thereafter. After 1 year on study the frequency of response assessments will be reduced to every 12 weeks (1 week). The tumor assessments will continue until the patient has radiologically-confirmed progression per the investigator, initiates a new anticancer therapy, dies, withdraws consent, or the study closes, whichever comes first.

(xi) Pharmacokinetic and ATA Assessments

Blood samples for PK and ATA will be collected throughout the study. Qualified or validated assays will be used to measure the concentrations of enfortumab vedotin ADC, total antibody (TAb), and MMAE in serum or plasma and assess ATA.

(xii) Biomarker Assessments

Samples for exploratory biomarkers will be collected at protocol-specified timepoints. Biomarker assessments will not be used for patient selection. Biomarker assessments in tumor tissue may include, but will not be limited to, tumor expression of Nectin-4 protein, messenger ribonucleic acid (mRNA) expression, markers of disease subtype, and markers of the immune microenvironment in tumor. Assessments in blood samples may include, but will not be limited to, markers of immune function, including abundance and phenotype of immune cell subsets, circulating tumor DNA (ctDNA), and abundance of cytokines. Methods of analysis may include immunohistochemistry (IHC), next generation sequencing, polymerase chain reaction (PCR), mutation and gene expression profiling, T-cell receptor beta chain sequencing, flow cytometry, and immunoassays.

(xiii) Safety Assessments

Safety assessments will be based on the information collected through the safety surveillance process and will include the data from recorded adverse events (AEs) including SAEs, recording of concomitant medication, physical examination findings, cardiac monitoring, and laboratory tests.

(xiv) Quality of Life Assessments

Patient reported outcomes (PRO) assessments will be used to obtain QoL information at protocol-specified timepoints. The following validated tools will be used; European Organisation for Research and Treatment of Cancer (EORTC) Quality of Life Questionnaire (QLQ-C30) and EuroQol 5-dimensions (EQ-5D).

(xv) Statistical Methods

(a) Analysis for Primary Endpoint

The primary endpoint of this study is the confirmed ORR per IRF. The ORR is defined as the proportion of patients with confirmed CR or PR according to RECIST Version 1.1. The ORR per IRF and its exact 2-sided 95% confidence interval (CI) using the Clopper-Pearson method (Clopper et al., Biometrika (1934); 26(4): 404-413) will be calculated.

There are 2 cohorts of CPI-treated patients in the study: Cohort 1) platinum-treated patients as defined in the study population above, and Cohort 2) platinum-naïve and cisplatin-ineligible patients also defined in the study population above. The primary endpoint will be analyzed separately for each cohort and may be analyzed for all patients, combining both Cohorts 1 and 2.

The primary analysis on Cohort 1 (platinum-treated patients) will be conducted when enrollment is completed in Cohort 1, and all patients in the cohort have been followed for at least 6 months, or have discontinued from study, or had 30 days safety follow-up after PD, whichever comes first. Analysis for Cohort 2 will occur at 4 timepoints: 1) at the time of analysis of Cohort 1, 2) when approximately 50 patients in Cohort 2 have had the opportunity to be followed for approximately 8 months from the first dose of enfortumab vedotin, 3) when approximately 70 patients in Cohort 2 have had the opportunity to be followed for approximately 8 months from the first dose of enfortumab vedotin, and 4) when all patients in Cohort 2 have had the opportunity to be followed for approximately 8 months from the first dose of enfortumab vedotin. All patients may also be analyzed at the time of analysis of Cohort 1 and/or Cohort 2.

The primary analysis of efficacy endpoints will be based on the full analysis set, including all patients who enrolled and received any amount of enfortumab vedotin. At the time of the interim analyses for Cohort 2, additional analyses of efficacy endpoints will be performed based on the efficacy evaluable set, including all patients in the full analysis set who started treatment with enfortumab vedotin at least 8 months before the analysis data cutoff.

(b) Sample Size

The study is designed to estimate the confirmed ORR in patients receiving enfortumab vedotin and to detect an improvement in the ORR compared with a historical response rate of 10%. Approximately 200 patients will be enrolled in this study to ensure collection of sufficient efficacy and safety data, including approximately 100 or more platinum-treated patients (Cohort 1), and up to approximately 100 platinum-naïve and cisplatin-ineligible patients (Cohort 2). Using the estimate of approximately 100 patients Cohort 1, the study will have 98% power to detect a 15% increase in ORR from 10% to 25% and 81% power to detect a 10% increase in ORR from 10% to 20%, at one-sided significant level of 0.025, based on exact methods using EAST®, Version 6.0, by Cytel Inc.

The confirmed ORR and 95% exact C1 in Cohort 2 will be summarized at 4 timepoints: 1) at the time of analysis of Cohort 1, 2) when approximately 50 patients in Cohort 2 have had the opportunity to be followed for approximately 8 months from the first dose of enfortumab vedotin, 3) when approximately 70 patients in Cohort 2 have had the opportunity to be followed for approximately 8 months from the first dose of enfortumab vedotin, and 4) when all patients treated in Cohort 2 have had the opportunity to be followed for approximately 8 months from the first dose of enfortumab vedotin.

For illustration purposes, Table 6 below is the summary of expected 95% CIs for Cohort 2 at various analysis timepoints, assuming a 30% observed ORR:

TABLE 6

| Number | Expected 95% |
|---|---|
| of | Confidence |
| Patients | Interval |
| N = 20 | 12%-54% |
| N = 50 | 18%-45% |
| N = 70 | 20%-42% |
| N = 100 | 21%-40% |

### 6.1.3 Objectives

Primary Objective

The primary Objective is to determine the antitumor activity of single-agent enfortumab vedotin as measured by confirmed ORR in patients with locally advanced or metastatic urothelial cancer who have previously received systemic therapy with a CPI and either previously received platinum-containing chemotherapy or are platinum-naïve and cisplatin-ineligible.

Secondary Objectives include:
To assess DOR,
To assess disease control rate (DCR),
To assess PFS,
To assess OS,
To assess the safety and tolerability of enfortumab vedotin,
To assess the pharmacokinetics (PK) of enfortumab vedotin, and
To assess the incidence of antitherapeutic antibodies (ATA).

Additional Objectives include:
To explore potential correlations between biomarkers and clinical outcomes, and
To evaluate the treatment effect of enfortumab vedotin on quality of life (QoL)

Endpoints

Primary Endpoint

The primary efficacy endpoint of this study is ORR (confirmed CR or PR per Response Evaluation Criteria in Solid Tumors (RECIST) Version 1.1) as determined by blinded independent central review (BICR) by an independent review facility (IRF).

Secondary Endpoints
DOR (confirmed CR or PR) per IRF
DCR₁₆ (disease control rate (CR, PR or SD) at 16 weeks) per IRF
PFS per IRF
ORR per investigator assessment
DOR per investigator assessment
DCR16 per investigator assessment
PFS per investigator assessment
OS
Type, incidence, severity, seriousness, and relatedness of AEs
Laboratory abnormalities
Selected plasma or serum PK parameters of enfortumab vedotin, MMAE, and total antibody (TAb)
Incidence of ATA to enfortumab vedotin

Additional Endpoints
Biomarkers of biological and clinical activity, including Nectin-4 expression
Patient reported outcomes (PRO) per the European Organisation for Research and Treatment of Cancer (EORTC) Quality of Life Questionnaire (QLQ-C30)
PRO per EuroQol 5-dimensions (EQ-5D), including health utility values, and visual analog scale

### 6.1.4 Study Plan

6.1.4.1 Summary of Study Design

This is a single-arm, open-label, multicenter trial designed to assess the efficacy and safety of enfortumab vedotin as a single agent in locally advanced or metastatic urothelial cancer patients who have previously received systemic therapy with a CPI. Patients must also either have received prior treatment with platinum-containing chemotherapy (Cohort 1) or received no prior platinum-containing or other chemotherapy and are ineligible for treatment with cisplatin at time of enrollment. (Cohort 2). Patients must have progressive disease (PD) during or following their most recent therapy. There are no limits for prior lines of therapy, including taxanes.

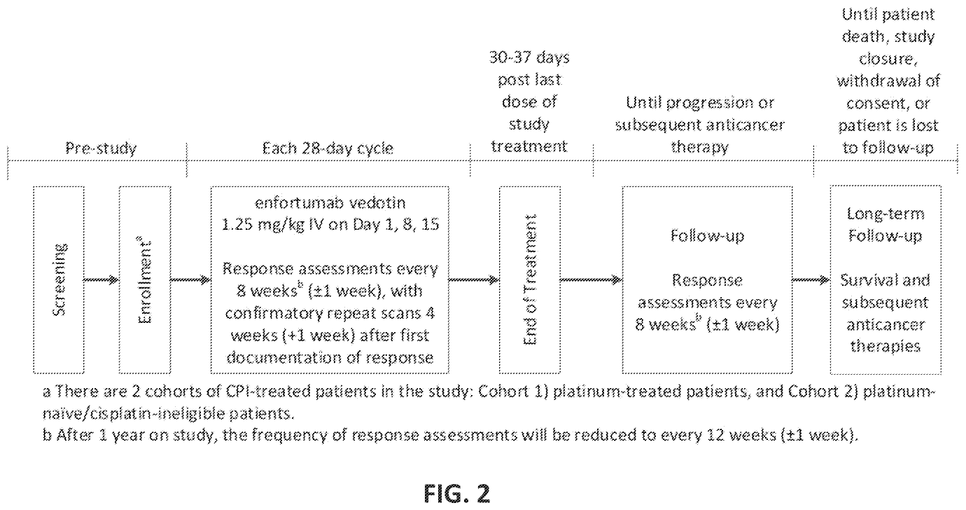

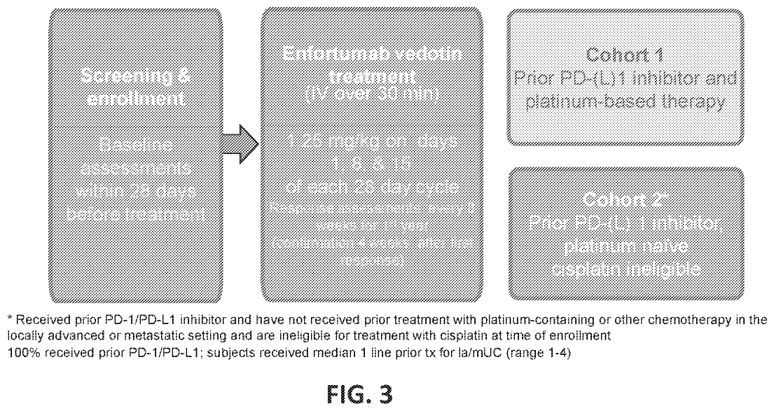

Enfortumab vedotin at a dose of 1.25 mg/kg will be administered as an intravenous (IV) infusion over approximately 30 minutes on Days 1, 8, and 15 of each 28-day cycle. Measures of anticancer activity will be assessed by computed tomography (CT scans with contrast, unless contraindicated) every 8 weeks (±1 week) timed from Cycle 1 Day 1 during treatment. After 1 year on study the frequency of response assessments will be reduced to every 12 weeks (1 week). Patients will continue to receive study treatment until disease progression, unacceptable toxicity, investigator decision, consent withdrawal, start of subsequent anticancer therapy, pregnancy, or study termination by the sponsor. After discontinuation of study treatment, patients will be followed every 8 weeks (1 week) for response assessments, Eastern Cooperative Oncology Group (ECOG) performance status, and physical exams. After 1 year on study the frequency of follow up visits including response assessments will be reduced to every 12 weeks (±1 week). Patients that have radiologically-confirmed disease progression (per RECIST 1.1 as determined by the investigator) or have begun subsequent anticancer therapy will be contacted every 8 weeks (±1 week) up to 1 year on study, and every 12 weeks (±1 week) thereafter to obtain information on subsequent anticancer therapy, and survival status until death, study closure, withdrawal of consent, or patient is lost to follow-up, whichever occurs first (see FIG. 2). The study will be closed 5 years after enrollment of the last patient, or when no patients remain in long-term follow-up, whichever occurs first. Additionally, the sponsor may terminate the study at any time.

On a periodic basis, an IDMC will monitor the safety of patients participating in this trial. The IDMC will be responsible for evaluating the results of safety analyses and will make recommendations to the sponsor. The IDMC may also request efficacy data, if needed, to evaluate risk/benefit before making recommendations. The IDMC will make recommendations to either continue the study unchanged, to modify the study, or to discontinue the study. If corneal AEs are observed in <15% of the first 60 enrolled patients (from Cohorts 1 and/or 2) and if the events are generally low grade or asymptomatic, the IDMC may make a recommendation to cease Cycle 2 Day 22 and/or Cycle 6 Day 22 slit lamp exams for the remaining patients if warranted based on review of the cumulative ocular safety data. The IDMC will communicate the recommendations to the sponsor. The final decision to act on the IDMC recommendations will be made by the sponsor. The IDMC recommended to cease Cycle 2 Day 22 and Cycle 6 Day 22 slit lamp exams in July 2018 and August 2019, respectively.

An ongoing real-time review of patient safety and SAEs will also be conducted by the sponsor's Drug Safety Department.

6.1.4.2 Discussion and Rationale for Study Design

This study will enroll patients who have previously received CPI therapy. Based on prior treatment patterns, taxanes or vinflunine are anticipated to be the next line of therapy to address post-CPI relapsed disease, but response rates are expected to be only 9%-13% (Bellmunt et al., N Engl J Med (2017); 376(11); 1015-26; Bellmunt et al., J Clin Oncol (2009); 27(27): 4454-61; Choueiri et al., Clin Onco (2012):1 30(5): 507-12.; McCaffrey et al., Curr Opin Urol (1997): 26(6): 556-63.). Data presented from a recent study of patients treated with docetaxel after platinum and CPI therapy showed a 10.5% ORR (n=2/19) (Drakaki et al., J Clin Oncol (2018): 36(6_suppl): Abstract 434). Recently, 10% has been used as the historical response rate expected for both platinum pre-treated second-line and cisplatin ineligible first-line patients (Balar et al., Lancet (2017): 389(10064): 67-76; Rosenberg et al., Lancet (2016): 387(10031): 1909-20.). Given that patients enrolled in the current study have a poor prognosis and represent an area of unmet medical need, the lower bound of the exact 95% confidence interval (CI) for ORR that excludes a historical response rate of 10% is considered to be a meaningful improvement over currently available therapies with the proposed enrollment of approximately 100 patients.

The primary endpoint of this study, confirmed ORR, is a direct measure of antitumor activity and is an acceptable surrogate endpoint (FDA Guidance for Industry “Clinical Trial Endpoints for the Approval of Cancer Drugs and Biologics”). To further assess the significance of ORR in this study, the durability of response will be evaluated as a secondary endpoint. Furthermore, standardized RECIST (Version 1.1, see Table 28) criteria (Eisenhauer et al., Eur J Cancer (2009); 45(2): 228-47) will be employed by investigators and the IRF to evaluate responses. Responses will be confirmed per IRF with repeat scans 4 weeks (±1 week) after initial documentation of response.

ORR may be evaluated in a single-arm study when it is defined as the sum of PR plus CR (FDA Guidance for Industry “Clinical Trial Endpoints for the Approval of Cancer Drugs and Biologics”). Moreover, to ensure consistent unbiased application of the RECIST criteria in this open-label study, all imaging assessments performed to confirm disease status at study entry and to assess responses during the study will be submitted to an independent third-party imaging core laboratory.

(i) Method of Assigning Patients to Treatment Groups

This is a single-arm study in which all patients will receive 1.25 mg/kg enfortumab vedotin administered as an IV infusion over approximately 30 minutes on Day 1, 8, and 15 of each 28-day treatment cycle.

(ii) Blinding

This is an open-label, single-arm study.

### 6.1.5 Study Population

All relevant medical and non-medical conditions should be taken into consideration when deciding whether this protocol is suitable for a particular patient. Patients must meet all of the enrollment criteria to be eligible for this study. Eligibility criteria may not be waived by the investigator and are subject to review in the event of a good clinical practice audit and/or health regulatory authority inspection.

Based upon the prevalence of high Nectin-4 expression in this population of metastatic urothelial cancer patients as previously established, this phase 2 study does not include an explicit eligibility requirement for a minimum level of Nectin-4 expression. However. Nectin-4 expression is assessed with tumor tissue collected at screening.

6.1.5.1 Inclusion Criteria

Patients must have histologically documented urothelial (previously known as transitional cell) carcinoma (i.e., cancer of the bladder, renal pelvis, ureter, or urethra). Patients with squamous differentiation or mixed cell types are eligible. Patients with resectable locally advanced disease are ineligible.

Patient must have received prior treatment with a CPI in the locally advanced or metastatic urothelial cancer setting. Patients who received CPI therapy in the neoadjuvant/adjuvant setting and had recurrent or progressive disease either during therapy or within 3 months of therapy completion are eligible. A CPI is defined as a PD-1 inhibitor or PD-L1 inhibitor (including, but not limited to: atezolizumab, pembrolizumab, durvalumab, avelumab, and nivolumab).

Patients must be one of the following:
Platinum-treated (Cohort 1): Patients who received prior treatment with platinum-containing chemotherapy defined as those who received platinum in the adjuvant/neoadjuvant setting and had recurrent or progressive disease within 12 months of completion OR received treatment with platinum in the locally advanced (defined as unresectable with curative intent) or metastatic setting; or
Platinum-naïve and cisplatin ineligible (Cohort 2): Patients who have not received prior treatment with platinum-containing or other chemotherapy in the locally advanced or metastatic setting and are ineligible for treatment with cisplatin at time of enrollment due to one of the following: ECOG performance status score of 2; impaired renal function (defined as creatinine clearance (CrCl)≥30 and <60 mL/min), or a ≥Grade 2 hearing loss. Patients who received platinum in the adjuvant/neoadjuvant setting and did not progress within 12 months of completion will be considered platinum-naïve.

Patients must have had progression or recurrence of urothelial cancer during or following receipt of most recent therapy.

Tumor tissue samples must be available for submission to the sponsor prior to study treatment.

Legally an adult according to local regulation at the time of signing informed consent, and minimum age of 18 years.

Patients must have measurable disease according to RECIST (Version 1.1) (Eisenhauer et al., Eur J Cancer(2009); 45(2): 22847.). Lesions in a prior radiation field must have progressed subsequent to radiotherapy to be considered measurable.

An ECOG performance status score of ≤1 for Cohort 1, or ≤2 for Cohort 2.

The following baseline laboratory data, assessed locally (no transfusions are permitted within 2 weeks prior to screening):
absolute neutrophil count ≥1.0×10⁹/L
platelet count ≥100×10⁹/L
hemoglobin ≥9 g/dL
serum bilirubin ≤1.5×upper limit of normal (ULN) or ≤3×ULN for patients with Gilbert's disease
CrCl ≥30 mL/min as measured by 24-hour urine collection or estimated by the Cockcroft-Gault criteria
alanine aminotransferase (ALT) and aspartate aminotransferase (AST)≤3×ULN

6.1.5.2 Exclusion Criteria

Ongoing sensory or motor neuropathy Grade ≥2.

Active central nervous system (CNS) metastases. Patients with treated CNS metastases are permitted on study if all the following are true.
CNS metastases have been clinically stable for at least 6 weeks prior to screening and baseline scans show no evidence of new or enlarged metastasis
If requiring steroid treatment for CNS metastases, the patient is on a stable dose
≤20 mg/day of prednisone or equivalent for at least 2 weeks
Patient does not have leptomeningeal disease

Ongoing clinically significant toxicity (Grade 2 or higher) associated with prior treatment (including systemic therapy, radiotherapy or surgery). Patients with ≤Grade 2 hypothyroidism or panhypopituitarism related to treatment with PD-1 and PD-L1 inhibitors may be enrolled. Patients on hormone replacement therapy may be enrolled if on a stable dose. Patients with ≥Grade 3 immunotherapy-related hypothyroidism or panhypopituitarism are excluded. Patients with immunotherapy related myocarditis, colitis, uveitis, or pneumonitis are excluded. Patients with other immunotherapy related adverse events requiring high doses of steroids (>20 mg/day of prednisone or equivalent) are excluded.

Prior enrollment in an enfortumab vedotin study or prior treatment with other MMAE-based ADCs.

History of another malignancy within 3 years before the first dose of study drug, or any evidence of residual disease from a previously diagnosed malignancy. Patients with nonmelanoma skin cancer, localized prostate cancer treated with curative intent with no evidence of progression, low-risk or very low-risk (per standard guidelines) localized prostate cancer under active surveillance/watchful waiting without intent to treat, or carcinoma in situ of any type (if complete resection was performed) are allowed.

Currently receiving systemic antimicrobial treatment for active infection (viral, bacterial, or fungal) at the time of first dose of enfortumab vedotin. Routine antimicrobial prophylaxis is permitted.

Patients with a positive hepatitis B surface antigen and/or antihepatitis B core antibody. Patients with a negative polymerase chain reaction (PCR) assay are permitted with appropriate antiviral prophylaxis.

Active hepatitis C infection or known human immunodeficiency virus (HIV) infection. Patients who have been treated for hepatitis C infection are permitted if they have documented sustained virologic response of ≥12 weeks.

Documented history of a cerebral vascular event (stroke or transient ischemic attack), unstable angina, myocardial infarction, or cardiac symptoms (including congestive heart failure) consistent with New York Heart Association Class III-IV (see 0) within 6 months prior to the first dose of enfortumab vedotin.

Radiotherapy or major surgery within 2 weeks prior to first dose of study drug.

Chemotherapy, biologics, investigational agents, and/or antitumor treatment with immunotherapy that is not completed 2 weeks prior to first dose of study drug.

Known hypersensitivity to enfortumab vedotin or to any excipient contained in the drug formulation of enfortumab vedotin (including histidine, trehalose dihydrate, and polysorbate 20).

Patients with active keratitis or corneal ulcerations. Patients with superficial punctate keratitis are allowed if the disorder is being adequately treated in the opinion of the investigator.

Other underlying medical condition that, in the opinion of the investigator, would impair the ability of the patient to receive or tolerate the planned treatment and follow-up.

Patients with uncontrolled diabetes. Uncontrolled diabetes is defined as hemoglobin A1c (HbA1c)≥8% or HbA1c 7—<8% with associated diabetes symptoms (polyuria or polydipsia) that are not otherwise explained.

Uncontrolled tumor-related bone pain or impending spinal cord compression. Patients requiring pain medication must be on a stable regimen at the time of enrollment (a minimum of 2 weeks).

### 6.1.6 Treatments

6.1.6.1 Treatments Administered

Patients in this study will receive enfortumab vedotin 1.25 mg/kg administered as an IV infusion over approximately 30 minutes on Days 1, 8, and 15 of every 28-day cycle.

6.1.6.2 Investigational Study Drug

(i) Description

Enfortumab vedotin is generated by conjugation of a chemical intermediate that contains both the MMAE and linker subunits, to cysteine residues of the antibody. The resulting ADC contains an average of 3.8 drug molecules per antibody. The enfortumab vedotin drug product is a sterile, preservative free, white to off-white lyophilized powder to be reconstituted for IV administration. Enfortumab vedotin is supplied in 30 mg single-dose vials.

(ii) Dose and Administration

Enfortumab vedotin at a dose of 1.25 mg/kg will be administered as an IV infusion over approximately 30 minutes on Days 1, 8, and 15 of every 28-day cycle. In the absence of infusion-related reactions (IRRs), the infusion rate for all patients should be calculated in order to achieve an approximate 30-minute infusion period. Enfortumab vedotin must not be administered as an IV push or bolus. Enfortumab vedotin should not be mixed with other medications. At least 1 week must elapse between doses of enfortumab vedotin.

Weight-based dosing is calculated using the patient's actual body weight. An exception to weight-based dosing is made for patients weighing greater than 100 kg; doses will be based on 100 kg for these individuals. The maximum dose permitted on this study is 125 mg.

Doses must be adjusted for patients who experience a ≥10% change in weight from baseline, or previous cycle. Patient weight must be measured during all relevant assessment windows as described in Table 26. Other dose adjustments for changes in body weight <10% are permitted per institutional standard.

The injection site should be monitored closely for redness, swelling, pain, and infection during and at any time after administration. Patients should be advised to report redness or discomfort promptly at the time of administration or after infusion. Institutional guidelines will be followed for the administration of chemotherapy agents and precautions taken to prevent extravasation per institutional standards and as described in “Chemotherapy and Biotherapy Guidelines and Recommendations for Practice” (Polovich et al., Oncology Nursing Society (2014): 155-70) and “Management of Chemotherapy Extravasation: ESMO-EONS Clinical Practice Guidelines” (Perez Fidalgo et al., Eur J Oncol Nurs (2012); 16(5); 528-34.).

The patient should be observed during administration of enfortumab vedotin and for at least 60 minutes following the infusion during the first 3 cycles. All supportive measures consistent with optimal subject care should be given throughout the study according to institutional standards.

(iii) Dose Modification

Intrapatient dose reduction to 1 mg/kg (dose level −1), and to 0.75 mg/kg (dose level −2) will be allowed depending on the type and severity of toxicity. Patients requiring a dose reduction may be re-escalated by 1 dose level (e.g., patients reduced to 0.75 mg/kg may only be re-escalated to 1 mg/kg) provided the toxicity does not require study drug discontinuation and has returned to baseline or ≤Grade 1. If the toxicity recurs, re-escalation will not be permitted. Patients with ≥Grade 2 corneal AEs will not be permitted to dose re-escalate.

Enfortumab vedotin should not be administered to patients with known CrCl <30 mL/min. Dose modification recommendations for enfortumab vedotin-associated toxicity are presented in Table 7 and Table 8.

If toxicities occur on Day 1 of any cycle and require the enfortumab vedotin dose to be held, then the start of the cycle may be delayed. If toxicities occur on Days 8 or 15 of any cycle and require the dose to be held >2 days, then the dose(s) must be eliminated, rather than delayed. If a patient only receives enfortumab vedotin on Day 1 and needs to skip Days 8 and 15, the patient could resume the next cycle as early as Day 22 (new Day 1) if the toxicity has resolved by that time.

Intrapatient dose reduction or delay for other enfortumab vedotin-associated toxicity is permitted at the discretion of the site investigator. Dose delays may last up to 8 weeks (2 cycles). Dose delays for patients who are responding to treatment may be extended beyond 8 weeks, if the patient's toxicity does not otherwise require permanent discontinuation. Patients may not receive other investigational drugs, radiotherapy (except palliative radiotherapy as described in 6.1.6.3(ii)), or systemic antineoplastic therapy during dose delays. If there is a dose delay, the schedule for response assessments will not be adjusted and will continue to be timed from Cycle 1 Day 1 during treatment.

TABLE 7
Recommended dose modifications for enfortumab vedotin-associated
hematologic toxicity

| Grade 1 | Grade 2 | Grade 3 | Grade 4 |
|---|---|---|---|
| Continue at same | Continue at same | Withhold dose until | Withhold dose until |
| dose level. | dose level. | toxicity is ≤ Grade 1 | toxicity is ≤ Grade 1 |
| | For Grade 2 | or has returned to | or has returned to |
| | thrombocytopenia, | baseline, then | baseline, then reduce |
| | withhold dose until | resume treatment at | dose by 1 dose level |
| | toxicity is ≤ Grade 1 | the same dose level | and resume |
| | or has returned to | or consider dose | treatment, or |
| | baseline, then resume | reduction by 1 dose | discontinue at the |
| | treatment at the same | level. Transfusions | discretion of the |
| | dose level. | or growth factors | investigator. |
| | | may be used as | Transfusions or |
| | | indicated per | growth factors may |
| | | institutional | be used as indicated |
| | | guidelines. | per institutional |
| | | | guidelines. |
| | | | For anemia, |
| | | | treatment |
| | | | discontinuation |
| | | | should be strongly |
| | | | considered. |

TABLE 8
Recommended dose modifications for enfortumab vedotin-associated
nonhematologic toxicity

| Grade 1 | Grade 2 | Grade 3 | Grade 4 |
|---|---|---|---|
| Continue at same | Continue at same | Withhold dose until | For Grade 4 AEs, |
| dose level. | dose level, except in | toxicity is ≤ Grade 1 or | discontinue |
| If ocular symptoms | the event of Grade 2 | has returned to | treatment.ª Grade 4 |
| and/or changes in | neuropathy or | baseline, then resume | vomiting and/or |
| vision are | corneal AEs. | treatment at the same | diarrhea that |
| identified, the | For Grade 2 | dose level or consider | improves to ≤ |
| patient should be | neuropathy or | dose reduction by 1 | Grade 2 within 72 |
| evaluated by a | corneal AEs, | dose level·ª | hours with |
| qualified | withhold dose until | For Grade 3 | supportive |
| optometrist or | toxicity is ≤ Grade | neuropathy or corneal | management does |
| ophthalmologist. | 1 or has returned to | AEs, discontinue | not require |
| | baseline, then | treatment. | discontinuation. |
| | resume treatment at | For Grade 3 | |
| | the same dose level. | hyperglycemia/elevated | |
| | For the second | blood glucose withhold | |
| | occurrence of Grade | enfortumab vedotin | |
| | 2 neuropathy or | treatment. Resume | |
| | corneal AEs, | treatment once | |
| | withhold dose until | hyperglycemia/elevated | |
| | toxicity is ≤ Grade | blood glucose has | |
| | 1, then reduce the | improved to ≤ Grade 2 | |
| | dose by 1 dose level | and patient is clinically | |
| | and resume | and metabolically | |
| | treatment. | stable. | |
| | If ocular symptoms | If ocular symptoms | |
| | and/or changes in | and/or changes in | |
| | vision are | vision are identified, | |
| | identified, the | the patient should be | |
| | patient should be | evaluated by a | |
| | evaluated by a | Grade 3 | |
| | qualified | qualified optometrist or | |
| | optometrist or | ophthalmologist. | |
| | ophthalmologist. | | |

^{a}Grade 3/4 electrolyte imbalances/laboratory abnormalities, except hyperglycemia, that are not associated with clinical sequelae and are corrected with supplementation/appropriate management within 72 hours of their onset do not require discontinuation (e.g., Grade 4 hyponatremia). Grade 3 rash that is not limiting self-care activities of daily living or associated with infection requiring systemic antibiotics does not require treatment interruption, provided symptoms are not severe and can be managed with supportive treatment.

See Section 6.1.6.4(i) for recommended management of infusion reactions. See Section 6.1.6.4(ii) for recommended management of hyperglycemia. See Section 6.1.6.4(iii) for recommended management of rash.

(a) Treatment Discontinuation Recommendations Related to Liver Safety

In the absence of an explanation for increased liver function tests (LFTs), such as viral hepatitis, preexisting or acute liver disease, or exposure to other agents associated with liver injury, the patient may be discontinued from the study treatment. The investigator may determine that it is not in the patient's best interest to continue study treatment.

Discontinuation of treatment should be considered if:
ALT or AST>8×ULN
ALT or AST>5×ULN for more than 2 weeks
ALT or AST>3×ULN and total bilirubin>2×ULN or international normalized ratio (INR)>1.5 (if INR testing is applicable/evaluated)
ALT or AST>3×ULN with the appearance of symptoms suggestive of liver injury (e.g., right upper quadrant pain or tenderness) and/or eosinophilia (>5%)

These treatment discontinuation recommendations are based on the FDA Guidance for Industry (Drug-Induced Liver Injury: Premarketing Clinical Evaluation. July 2009). The recommendations are a basic guide to the investigator based on accumulated clinical experience with drugs in development, and are not specific to clinical experience with enfortumab vedotin.

See 0 for recommended liver safety monitoring and assessment criteria in patients with Grade 2 or greater elevations in ALT, AST, or bilirubin.

6.1.6.3 Concomitant Therapy

All concomitant medications and blood products will be recorded from Day 1 (predose) through the safety reporting period (30 days after the last study treatment). Any concomitant medication given for a study protocol-related adverse event should be recorded from the time of informed consent.

(i) Required Concomitant Therapy

There are no required concomitant therapies.

(ii) Allowed Concomitant Therapy

Concomitant chronic prednisone (or equivalent) may be used at a dose of ≤20 mg/day. Higher doses of prednisone (or equivalent) are permitted for limited duration to treat acute conditions that arise during the study as medically indicated. The use of anti-emetics are permitted. Premedications for IRRs per Section 6.1.6.4(i) are permitted.

Therapy to manage enfortumab vedotin-associated toxicity as recommended in Section 6.1.6.2(iii) are permitted, including growth factors, and transfusions.

Patients who are receiving strong CYP3A4 inhibitors or P-glycoprotein (P-gp) inhibitors concomitantly with enfortumab vedotin should be closely monitored for adverse reactions.

Routine prophylaxis with vaccines is permitted; it is recommended that vaccines used do not contain live micro-organisms.

Palliative radiotherapy on a non-target bone lesion that is not progressing is allowed after 3 cycles of treatment; must be administered after the initial response assessment and repeat scans described in Section 6.1.7.2. This will not be considered a subsequent anticancer therapy, but must not interfere with the assessment of tumor target lesions. Treatment with enfortumab vedotin should be interrupted during palliative radiotherapy.

Patients with a positive hepatitis B surface antigen and/or antihepatitis B core antibody and a negative PCR assay at baseline should receive appropriate antiviral prophylaxis or regular surveillance monitoring as per local or institutional guidelines.

(iii) Prohibited Concomitant Therapy

Patients may not receive other investigational drugs, radiotherapy (except palliative radiotherapy as described in Section 5.3.2), or systemic anti-neoplastic therapy during the treatment period. Patients who receive prohibited concomitant therapy must be discontinued from the study.

6.1.6.4 Management of Adverse Reactions

(i) Management of Infusion Reactions

An IRR may occur during the infusion of study treatment. The infusion should be administered at a site properly equipped and staffed to manage anaphylaxis should it occur. All supportive measures consistent with optimal patient care should be given throughout the study according to institutional standards. Supportive measures may include administering medications for IRRs.

Patients who have experienced an IRR may be premedicated for subsequent infusions. Premedication may include pain medication (e.g., acetaminophen or equivalent), an antihistamine (e.g., diphenhydramine hydrochloride), and a corticosteroid administered 30-60 minutes prior to each infusion or according to institutional standards. Should a patient experience IRRs in the setting of premedication, continued treatment with enfortumab vedotin must be discussed with the medical monitor prior to the next planned dose.

If anaphylaxis occurs, study treatment administration should be immediately and permanently discontinued.

(ii) Management of Hyperglycemia

Investigators should monitor blood glucose levels and are advised to perform additional assessments if any symptoms of hyperglycemia are observed, including a thorough evaluation for infection. In addition, if steroids are used to treat any other condition, blood glucose levels may require additional monitoring. If elevated blood glucose levels are observed, patients should be treated according to local standard of care and referral to endocrinology may be considered.

Patients, especially those with a history of or ongoing diabetes mellitus or hyperglycemia, should be advised to immediately notify their physician if their glucose level becomes difficult to control or if they experience symptoms suggestive of hyperglycemia such as frequent urination, increased thirst, blurred vision, fatigue, and headache.

Patients who enter the study with an elevated HbA1c (≥6.5%) at baseline should be referred to an appropriate provider during Cycle 1 for glucose management. Blood glucose should be checked prior to each dosing and dose should be withheld for blood glucose >250 mg/dL (Grade 3 or higher), regardless of relatedness to enfortumab vedotin. Dosing may continue once the patient's blood glucose has improved to ≤250 mg/dL (≤Grade 2) and the patient is clinically and metabolically stable. Patients with blood glucose >500 mg/dL (Grade 4) considered unrelated to enfortumab vedotin may continue dosing once the patient's blood glucose has improved to ≤250 mg/dL (≤Grade 2) and the patient is clinically and metabolically stable. Blood glucose >500 mg/dL (Grade 4) considered related to enfortumab vedotin requires treatment discontinuation. If a patient experiences new onset diabetes mellitus, evaluate patients with a metabolic panel, urine ketones, glycosylated hemoglobin, and C-peptide to assess for new onset type 1 diabetes in the setting of prior CPI.

(iii) Management of Rash

In the phase 1 study (Study ASG-22CE-13-2), rash and similar dermatologic AEs were common among patients treated with enfortumab vedotin, and were seen more frequently at the highest dose. Although the exact etiology of dermatologic toxicities associated with enfortumab vedotin is unclear at this time, due to the expression of Nectin-4 in the skin, rash may be an on-target toxicity. The most common dermatological AEs reported in ASG-22CE-13-2 were drug eruption, rash, skin exfoliation, skin pigmentation disorder, and rash maculo-papular. Most occurred during Cycle 1, and some were associated with pruritus. Almost all were mild, with the exception of two events of rash reported as Grade 3 in the 1 mg/kg dose group. None required discontinuation of study drug and one event of Grade 1 papular rash at 1.25 mg/kg required a dose reduction. Mild rash related to enfortumab vedotin should be treated using local supportive care as needed. Topical corticosteroids have been used along with antihistamines for pruritus as needed. Grade 3 rash that is not limiting self-care activities of daily living or associated with infection requiring systemic antibiotics does not require treatment interruption, provided symptoms are not severe and can be managed with supportive treatment.

6.1.6.5 Treatment Compliance

Study drug administration will be performed by study site staff and documented in source documents and the CRF.

### 6.1.7 Study Assessments

6.1.7.1 Screening/Baseline Assessments

Only patients who meet all inclusion and exclusion criteria specified in Section 6.1.5 will be enrolled in this study. Enrollment status and date will be recorded in CRF.

Patient medical history includes a thorough review of significant past medical history, current conditions, tobacco history, any treatment for prior malignancies and response to prior treatment, and any concomitant medications.

A complete eye examination, brain scan, bone scan, CT scan with contrast for baseline response efficacy assessment, INR/PT/PTT, serology for hepatitis B and C, urinalysis with reflexive microscopic analysis, HbA1c, and pregnancy test (either urine or serum, for females of childbearing potential) are required for all patients at screening.

6.1.7.2 Response/Efficacy Assessments

Measures of anticancer activity will be assessed by CT scans with contrast approximately every 8 weeks (±1 week). After 1 year on study, response assessments will be reduced to every 12 weeks (±1 week). The schedule of response assessments should not be adjusted for dose delays/interruptions or other reasons for changes in the timing of a patient's study activities; timepoints for response assessments should be calculated from Cycle 1 Day 1 during treatment. For patients who cannot receive CT scans with contrast, other acceptable imaging methods are detailed in 6.1.13. For brain scans, MRI with gadolinium is the preferred method of assessment; however, other imaging methods are detailed in 6.1.13 if contrast is contraindicated. Patients must be evaluated using the same imaging method throughout the study for efficacy assessments. CT scans with contrast at the minimum will include the chest, abdomen, and pelvis. Other regions should be scanned if the patient has known or suspected disease in that region. Brain and/or bone scans should also be repeated at response assessment timepoints if bone metastases were identified at baseline, or if metastasis is known or suspected. Responses (CR or PR) will be confirmed with repeat scans 4 weeks (+1 week window) after first documentation of response. The schedule for response assessments should not be adjusted after the confirmatory scan (e.g., CR at Week 8, confirmatory scans at Week 12, next assessment due at Week 16). Tumor imaging should also be performed whenever disease progression is suspected.

Patients who discontinue study treatment for reasons other than objective disease progression by RECIST Version 1.1 (see) will continue to receive CT scans with contrast weeks (±1 week) after the previous response assessment scan and every 8 weeks (±1 week) following the previous scan thereafter. After 1 year on study the frequency of response assessments will be reduced to every 12 weeks (±1 week). The tumor assessments will continue until the patient has radiologically-confirmed disease progression per RECIST as determined by the investigator, initiates a new anticancer therapy, dies or withdraws consent, or the study closes, whichever comes first. The determination of antitumor activity will be based on confirmed objective response assessments as defined by RECIST Version 1.1 (see Table 28) (Eisenhauer et al., Eur J Cancer (2009):45(2): 228-47.). Patients who do not have at least 2 (initial response and confirmation scan) post-baseline response assessments will be counted as non-responders. The investigator will make treatment decisions based on site assessments of scans by RECIST. Clinical response of CR, PR, SD, or PD will be determined at each assessment. Response and progression will also be assessed by an IRF.

Survival status will be updated every 8 weeks (1 week) after EOT (or 8 weeks from previous protocol visit, whichever is later) until death, study closure, or withdrawal of consent, whichever occurs first. After 1 year on study the frequency of survival status updates will be reduced to every 12 weeks (±1 week).

Patients' clinical data must be available for CRF source verification. Tumor images will be submitted to a central imaging lab.

6.1.7.3 Pharmacokinetic and ATA Assessments

Blood samples for PK and ATA will be collected throughout the study per the sample collection schedule provided in Table 9. Validated or qualified assays will be used to measure the concentrations of enfortumab vedotin ADC, TAb, and MMAE in serum or plasma. PK samples will be collected and archived for possible analysis of other enfortumab vedotin-related species, such as circulating metabolites of MMAE. A qualified assay will be used to determine the levels of ATA in serum.

Refer to the Central Laboratory Manual for information on collection, processing, storage, and shipment of sample.

6.1.7.4 Biomarker Studies

Samples for exploratory biomarkers will be collected at protocol-specified timepoints (see Table 9 and Table 26). Biomarker assessments will not be used for patient selection.

Methods of analysis may include IHC, next generation sequencing, PCR, mutation and gene expression profiling; T-cell receptor beta chain sequencing, flow cytometry and immunoassays.

(i) Biomarkers in Blood

The primary effects of enfortumab vedotin on urothelial carcinoma tumor cells may lead to changes in the activation state of local, tumor-associated and peripheral immune cells. Biomarker assessments in blood samples may include but may not be limited to: markers of immune function, including abundance and phenotype of immune cell subsets, circulating tumor DNA (ctDNA), and abundance of cytokines. These may provide insight into treatment-related changes in activation state of peripheral immune system associated with enfortumab vedotin-induced tumor cell death.

TABLE 9
Pharmacokinetic, ATA, and biomarker blood sample collection timepoints

| | | | | | Blood | | | | | | |
|---|---|---|---|---|---|---|---|---|---|---|---|
| | | | | | | | Biomarkers | | | | |
| | | | | | | | Plasma | | | PBMC | |
| | Study | | | | | | | Research | Research | Immuno- | |
| | Day | Time | Window | Relative Time | PK | ATA | Cytokines | 1 | 2 | phenotypin | Research |
| Cycles | Day 1 | Pre-dose | within 24 hr | START of infusion | X | X | X | X | X | X | X |
| 1 and 2 | | End of | within 15 min | END of infusion | X | | | | | | |
| | | infusion | | | | | | | | | |
| | Day 3 | 48 hr | ±24 hr | END of Day 1 infusion | X | | X^{a} | | X^{a} | X^{a} | X^{a} |
| | Day 8 | Pre-dose | within 24 hr | START of infusion | X | | X^{a} | X^{a} | X^{a} | X^{a} | X^{a} |
| | | End of | within 15 min | END of infusion | X | | | | | | |
| | | infusion | | | | | | | | | |
| | Day 15 | Pre-dose | within 24 hr | START of infusion | X | | X^{a} | | X^{a} | X^{a} | X^{a} |
| | | End of | within 15 min | END of infusion | X | | | | | | |
| | | infusion | | | | | | | | | |
| | Day 17 | 48 hr | −24 hr/+48 hr | END of Day 15 | X | | | | | | |
| | | | | infusion | | | | | | | |
| | Day 22 | 168 hr | ±48 hr | END of Day 15 | X | | | | | | |
| | | | | infusion | | | | | | | |
| Subsequent | Day 1 | Pre-dose | within 24 hr | START of infusion | X^{b} | X^{b} | X^{c} | X^{c} | X^{c} | X^{c} | X^{c} |
| dosing | | | | | | | | | | | |
| cycles | | | | | | | | | | | |

| | | | | | | | |
|---|---|---|---|---|---|---|---|
| End of Treatment (within 30-37 days of last dose) | X | X | X | X | X | X | X |

^{a}Cycle 1 only
^{b}Cycles 3 and 4 and every even-numbered cycle thereafter
^{c}Cycles 3 and 4 only

(ii) Biomarkers in Pre-Treatment Tumor Tissue

To better understand relationships between pre-treatment urothelial carcinoma biological characteristics, and patient outcome, submission of a tumor block or freshly sectioned, unstained charged slides (at least 10 slides are mandatory unless prior approval is obtained from the sponsor) of pretreatment tumor tissue are required. Either archival tissue or pre-treatment fresh tumor tissue (obtained from a fresh biopsy) is acceptable. See the Laboratory Manual for details.

Biomarker assessments in tumor tissue may include, but not be limited to:
Tumor expression of Nectin-4 protein
Messenger ribonucleic acid (mRNA) expression
Markers of disease subtype (e.g., The Cancer Genome Atlas (TCGA) subtypes)
Tumor mutational burden
Markers of the tumor immune microenvironment

6.1.7.5 Biospecimen Repository

In the US only, for patients who provide additional consent, remaining de-identified unused blood and/or tissue will be retained by the sponsors and used for future research, including but not limited to the evaluation of targets for novel therapeutic agents, the biology of ADC sensitivity and resistance mechanisms, and the identification of biomarkers for ADCs. Blood and tissue samples donated for future research will be retained for a period of up to 25 years. If additional consent is not provided, any remaining biological samples will be destroyed following study completion and when the samples are no longer required to be maintained for potential regulatory submissions.

6.1.7.6 Quality of Life

Two validated tools will be used: the QLQ-C30, and the EQ-5D.

If possible, PRO assessments should be completed before any other procedures at the study visits noted in the schedule of events (Table 26).

(i) EORTC Core Quality of Life Questionnaire, QLQ-C30

The QLQ-C30 was developed to measure aspects of QoL pertinent to patients with a broad range of cancers who are participating in clinical trials (Aaronson et al., J Natl Cancer Inst (1993); 85(5): 365-76, Sneeuw et al., J Clin Epidemiol (1998); 51(7): 617-31.). The current version of the core instrument (QLQ-C30, Version 3) is a 30-item questionnaire consisting of the following:
5 functional domains (physical, role, cognitive, emotional, social):
3 symptom scales (fatigue, pain, nausea & vomiting):
Single items for symptoms (shortness of breath, loss of appetite, sleep disturbance, constipation, diarrhea) and financial impact of the disease; and
2 global items (health, overall QoL).

(ii) EuroQol-5 Dimensions

The EQ-5D is a standardized instrument developed by the EuroQol Group for use as a generic, preference-based measure of health outcomes. It is applicable to a wide range of health conditions and treatments and provides a simple descriptive profile and a single index value for health status. The EQ-5D is a 5-item self-reported measure of functioning and well-being, which assesses 5 dimensions of health, including mobility, self-care, usual activities, pain/discomfort, and anxiety/depression. Each dimension comprises 3 levels (no problems, some/moderate problems, extreme problems). A unique EQ-5D health state is defined by combining 1 level from each of the 5 dimensions. This questionnaire also records the respondent's self-rated health status on a vertical graduated (0 to 100) visual analogue scale. Responses to the 5 items will also be converted to a weighted health state index (utility score) based on values derived from general population samples. The EQ-5D is recommended for use in cost-effectiveness analyses commonly employed in health technology assessments by the Washington Panel on Cost Effectiveness in Health and Medicine (Gold et al., Med Care 34(1996): (12 Suppl): DS197-9.).

6.1.7.7 Safety Assessments

The assessment of safety during the course of this study will consist of the surveillance and recording of AEs including SAEs, recording of concomitant medication, and measurements of protocol-specified physical examination findings, cardiac monitoring, and laboratory tests.

Safety will be monitored over the course of the study by an IDMC as described in Section 6.1.8.3(x).
(i) Adverse Events
(a) About Adverse Event

Adverse Event

According to the International Council for Harmonisation (ICH) E2A guideline Definitions and Standards for Expedited Reporting, and 21 Code of Federal Regulations (CFR) 312.32, Investigational New Drug (IND) Safety Reporting, an AE is any untoward medical occurrence in a patient or clinical investigational subject administered a medicinal product and which does not necessarily have a causal relationship with this treatment.

The following information should be considered when determining whether or not to record a test result, medical condition, or other incident on the Adverse Events and Pre-existing Conditions CRF:
From the time of informed consent through the day prior to study Day 1, only study protocol-related AEs should be recorded. A protocol-related AE is defined as an untoward medical event occurring as a result of a protocol mandated procedure.
All medical conditions present or ongoing predose on study Day 1 should be recorded.
All AEs (regardless of relationship to study drug) should be recorded from study Day 1 (pre-dose) through the end of the safety reporting period (see Section 6.1.7.7(i)(c)). Complications that occur in association with any procedure (e.g., biopsy) should be recorded as AEs whether or not the procedure was protocol mandated.
Changes in medical conditions and AEs, including changes in severity, frequency, or character, during the safety reporting period should be recorded.
In general, an abnormal laboratory value should not be recorded as an AE unless it is associated with clinical signs or symptoms, requires an intervention, results in a SAE, or results in study termination or interruption/discontinuation of study treatment. When recording an AE resulting from a laboratory abnormality, the resulting medical condition rather than the abnormality itself should be recorded (e.g., record “anemia” rather than “low hemoglobin”).

Serious Adverse Events

An AE should be classified as an SAE if it meets one of the following criteria:
Fatal: AE resulted in death
Life threatening: The AEs placed the patient at immediate risk of death. This classification does not apply to an AE that hypothetically might cause death if it were more severe.
Hospitalization: The AE resulted in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Pre-planned hospitalizations for therapeutic, diagnostic, or surgical procedures of the underlying cancer or study target disease that did not worsen during the clinical trial need not be captured as SAEs.
Disabling/incapacitating: An AE that resulted in a persistent or significant incapacity or substantial disruption of the patient's ability to conduct normal life functions.
Congenital anomaly or birth defect: An adverse outcome in a child or fetus of a patient exposed to the molecule or study treatment regimen before conception or during pregnancy.
Medically significant: The AE did not meet any of the above criteria, but could have jeopardized the patient and might have required medical or surgical intervention to prevent one of the outcomes listed above or involves suspected transmission via a medicinal product of an infectious agent.

Adverse Event Severity

AE severity should be graded using the National Cancer Institute's Common Terminology Criteria for Adverse Events (NCI CTCAE), Version 4.03. These criteria are provided in the study manual.

AE severity and seriousness are assessed independently. ‘Severity’ characterizes the intensity of an AE. ‘Serious’ is a regulatory definition and serves as a guide to the sponsor for defining regulatory reporting obligations (see definition for SAEs, above).

Relationship of the Adverse Event to Study Treatment

The relationship of each AE to enfortumab vedotin should be evaluated by the investigator using the following criteria:
Related: There is evidence to suggest a causal relationship between the drug and the AE, such as:
   A single occurrence of an event that is uncommon and known to be strongly associated with drug exposure (e.g., angioedema, hepatic injury, Stevens-Johnson Syndrome)
   One or more occurrences of an event that is not commonly associated with drug exposure, but is otherwise uncommon in the population exposed to the drug (e.g., tendon rupture)
Unrelated: Another cause of the AE is more plausible (e.g., due to underlying disease or occurs commonly in the study population), or a temporal sequence cannot be established with the onset of the AE and administration of the study treatment, or a causal relationship is considered biologically implausible

(b) Procedures for Eliciting and Recording Adverse Events

Investigator and study personnel will report all AEs and SAEs whether elicited during patient questioning, discovered during physical examination, laboratory testing and/or other means by recording them on the CRF and/or SAE form, as appropriate.

Eliciting Adverse Events

An open-ended or non-directed method of questioning should be used at each study visit to elicit the reporting of AEs.

Recording Adverse Events

The following information should be recorded on the Adverse Events and Pre-existing Conditions CRF:
Description including onset and resolution dates
Whether it met SAE criteria
Severity
Relationship to study treatment or other causality
Outcome

Diagnosis vs Signs or Symptoms

In general, the use of a unifying diagnosis is preferred to the listing out of individual symptoms. Grouping of symptoms into a diagnosis should only be done if each component sign and/or symptom is a medically confirmed component of a diagnosis as evidenced by standard medical textbooks. If any aspect of a sign or symptom does not fit into a classic pattern of the diagnosis, report the individual symptom as a separate adverse event.

Important exceptions for this study are adverse reactions associated with the infusion of study drug. For IRRs, record the NCI CTCAE term of ‘infusion related reaction’ with an overall level of severity (per NCI CTCAE). In addition, record each sign or symptom of the reaction as an individual AE. If multiple signs or symptoms occur with a given infusion-related event, each sign or symptom should be recorded separately with its level of severity.

Recording Serious Adverse Events

For SAEs, record the event(s) on both the CRF and an SAE form.

The following should be considered when recording SAEs:
Death is an outcome of an event. The event that resulted in the death should be recorded and reported on both an SAE form and CRF.
For hospitalizations, surgical, or diagnostic procedures, the illness leading to the surgical or diagnostic procedure should be recorded as the SAE, not the procedure itself. The procedure should be captured in the narrative as part of the action taken in response to the illness.

Progression of the Underlying Cancer

Do not report radiographic signs of disease progression (e.g., “tumor progression” or “metastases”) as an AE (this data is captured in the efficacy assessment). Report the symptoms and signs of disease progression (e.g., “fatigue”, “dyspnea”) as AEs; do not report disease progression as the AE term.

For disease progression with a fatal outcome, report the immediate cause of death as the event term. If the immediate cause of death cannot be determined and it is thought by the investigator to be related to disease progression, report the AE using the term ‘disease progression’ with a fatal outcome.

Pregnancy

Notification to Drug Safety: Complete a Pregnancy Report Form for all pregnancies that occur from the time of first study drug dose until 6 months after the last dose of study drug(s) including any pregnancies that occur in the partner of a male study patient. Only report pregnancies that occur in a male patient's partner if the estimated date of conception is after the male patient's first study drug dose. Email or fax to the sponsor's Drug Safety Department within 48 hours of becoming aware of a pregnancy. All pregnancies will be monitored for the full duration; all perinatal and neonatal outcomes should be reported. Infants should be followed for a minimum of 8 weeks.

Collection of data on the CRF: All pregnancies (as described above) that occur within 30 days of the last dose of study drug(s) will also be recorded on the Adverse Events and Pre-Existing Conditions CRF.

Abortion, whether accidental, therapeutic, or spontaneous, should be reported as an SAE. Congenital anomalies or birth defects, as defined by the ‘serious’ criterion above (see definitions Section 6.1.7.7(i)(a)) should be reported as SAEs.

Corneal Adverse Events

Corneal ulcer or keratitis AEs≥Grade 2 should be graded within their respective categories. Grade 1 corneal ulcer or keratitis AEs should be graded per “Eye disorders—Other, specify” criteria. Other corneal AEs should be recorded and graded per “Eye disorders—Other, specify” criteria.

Adverse Events of Possible Hepatic Origin

If an AE is accompanied by increases in LFT values (e.g., AST, ALT, bilirubin, etc.) or is suspected to be due to hepatic dysfunction, see 0 for detailed information on recommended monitoring and assessment of liver abnormalities. See Section 6.1.6.2(iii)(a) for treatment discontinuation recommendations related to hepatic safety.

Patients with AEs of hepatic origin accompanied by LFT abnormalities should be carefully monitored.

(c) Reporting Periods for Adverse Events and Serious Adverse Events

The safety reporting period for all AEs and SAEs is from study Day 1 (predose) through 30 days after the last study treatment. However, all study protocol-related AEs are to be recorded from the time of informed consent. All SAEs that occur after the safety reporting period and are considered study treatment-related in the opinion of the investigator should also be reported to the sponsor.

SAEs will be followed until significant changes return to baseline, the event stabilizes (recovering/resolving) or is no longer considered clinically significant by the investigator, or the patient dies or withdraws consent. All non-serious AEs will be followed through the safety reporting period. Certain non-serious AEs of clinical interest may be followed (including collection of relevant concomitant medications) until resolution, return to baseline, study closure, or the events become chronic to the extent that they are adequately characterized.

(d) Serious Adverse Events Require Immediate Reporting

Within 24 hours of observing or learning of an SAE, investigators are to report the event to the sponsor, regardless of the relationship of the event to the study treatment regimen.

For initial SAE reports, available case details are to be recorded on an SAE form. At a minimum, the following should be included:
Patient number
Date of event onset
Description of the event
Study treatment, if known

The completed SAE form and SAE Fax Cover Sheet are to be emailed or faxed to the study sponsor's Drug Safety Department or designee within 24 hours (see email or fax number(s) specified on the SAE report form).

Relevant follow-up information is to be submitted to the study sponsor as soon as it becomes available.

(ii) Clinical Laboratory Tests

Samples will be drawn for central and local labs.

Local laboratory testing will include institutional standard tests for study eligibility, evaluating safety, and making clinical decisions. Local laboratory testing should be performed on all dosing days. All local laboratory results must be reviewed prior to study drug administration in order to determine whether to proceed with dosing or whether dose modification is required.

The following laboratory assessments will be performed by the central lab to evaluate safety at scheduled timepoints (see Table 26) during the course of the study:
The serum chemistry panel is to include the following tests: albumin, alkaline phosphatase, ALT, AST, bicarbonate, blood urea nitrogen, calcium, creatinine, chloride, glucose, lactate dehydrogenase (LDH), phosphorus, potassium, sodium, total bilirubin, amylase, lipase, and uric acid.
The CBC with differential is to include the following tests: white blood cell count with five-part differential (neutrophils, lymphocytes, monocytes, eosinophils, and basophils), platelet count, hemoglobin, and hematocrit.

The following laboratory assessment(s) will be performed by local laboratories at scheduled timepoints (see Table 26) during the course of the study:
CrCl at baseline using the Cockcroft-Gault criteria or 24-hour urine collection
Standard urinalysis (with reflexive microscopy)
INR/PT/PTT
A serum or urine beta human chorionic gonadotropin (β-hCG) pregnancy test for females of childbearing potential
HbA1c
Serology for hepatitis B surface antigen and antihepatitis B core antibody
Serology for antihepatitis C antibody. If positive, follow up with PCR testing

(iii) Physical Examination Including Weight

Physical examinations should include assessments of the following body parts/systems: abdomen, extremities, head, heart, lungs, neck, and neurological. Height will be collected at the Baseline visit. Weight will be collected at specified timepoints (see Table 26), but does not need to be collected at visits following EOT.

(iv) Vital Signs

Vital sign measurements will be performed to include heart rate (bpm), diastolic and systolic blood pressure (mmHg), and temperature. Vital sign values will be recorded, and any diagnosis associated with clinically significant abnormal vital signs will be recorded as an adverse event or pre-existing condition.

(v) ECOG Performance Status

ECOG performance status (Table 27) will be evaluated at protocol-specified timepoints.

(vi) Cardiac Monitoring

ECGs will be conducted at baseline and at the EOT visit. Additional ECGs should be conducted if clinically indicated. Routine 12-lead ECGs will be performed after the patient has been in a supine position for at least 5 minutes. The ECG assessments should be performed prior to obtaining the PK and biomarker samples if possible.

(vii) Eye Examination

Patients will have a complete eye examination at baseline performed by a qualified ophthalmologist or optometrist, including but not limited to: uncorrected, corrected and best corrected visual acuity, slit lamp, tonometry examination, and dilated fundus examination. EOT slit lamp examinations are required for all patients who experience corneal adverse events during the study. EOT slit lamp examinations must be performed ≥4 weeks from last dose. Additional eye examinations are to be conducted as clinically indicated.

6.1.7.8 Post-Treatment Assessments

(i) Follow-Up Assessments

Patients who discontinue study treatment will continue to receive physical exams (no weight collection is required), ECOG assessment, and response assessments every 8 weeks (±1 week) after the previous response assessment scan and every 8 weeks (±1 week) following the previous visit thereafter until radiologically-confirmed disease progression (per RECIST as determined by the investigator), initiation of a new anticancer therapy, patient death, study closure, or withdrawal of consent, whichever comes first. After 1 year on study the frequency of follow-up visits will be reduced to every 12 weeks (1 week).

(ii) Long-Term Follow-Up Assessments

After radiologically-confirmed progression (per RECIST as determined by the investigator) or initiation of a new anticancer therapy, patients will be contacted every 8 weeks (1 week) after EOT (or 8 weeks from previous protocol visit, whichever is later) to obtain information on subsequent anticancer therapy and survival status. Long-term follow-up will continue until patient death, study closure, withdrawal of consent, or patient is lost to follow-up, whichever occurs first. After 1 year on study the frequency of long-term follow-up visits will be reduced to every 12 weeks (1 week).

6.1.7.9 Appropriateness of Measurements

The safety measures that will be used in this trial are considered standard procedures for evaluating the potential adverse effects of study medications.

The determination of antitumor activity will be based on confirmed objective response assessments as defined by RECIST Version 1.1 (see Table 28) (Eisenhauer et al., Eur J Cancer (2009); 45(2): 228-47.) and treatment decisions by the Investigator will be based on these assessments. These criteria are considered standard in oncological practice for this type of neoplasm, and the intervals of evaluation in this protocol are appropriate for disease management.

Immunogenicity is commonly assessed for biologics; therefore, standard tests will be performed to detect the possible presence of specific antibodies to enfortumab vedotin.

Pharmacokinetic assessments are also common in clinical studies to help characterize dose-exposure-response relationships.

Exploratory biomarker measurements in peripheral blood samples enable correlation with PK assessments and are common in clinical studies. Assessments conducted on pretreatment tumor tissue are similarly common. Both peripheral blood and tumor biomarker samples will be assessed using commonly employed, standard tests.

### 6.1.8 Data Analysis Methods

6.1.8.1 Determination of Sample Size

The study is designed to estimate the confirmed ORR in patients receiving enfortumab vedotin and to detect an improvement in the ORR compared with a historical 10% response rate. The rationale for the historical response rate is provided in Section 6.1.4.2.

Approximately 200 patients will be enrolled in this study to ensure collection of sufficient efficacy and safety data, including approximately 100 or more platinum-treated patients as defined in the eligibility criteria in Section 6.1.5.1 (Cohort 1), and up to approximately 100 platinum-naïve and cisplatin-ineligible patients also defined in the eligibility criteria in Section 6.1.5.1 (Cohort 2). Using the estimate of approximately 100 patients in Cohort 1, the study will have 98% power to detect a 15% increase in ORR from 10% to 25% and 81% power to detect a 10% increase in ORR from 10% to 20%, at one-sided significance level of 0.025, based on exact methods using EAST®, Version 6.0, by Cytel Inc.

The confirmed ORR and 95% exact C1 in Cohort 2 will be summarized at 4 timepoints: 1) at the time of analysis of Cohort 1, 2) when approximately 50 patients in Cohort 2 have had the opportunity to be followed for approximately 8 months from the first dose of enfortumab vedotin, 3) when approximately 70 patients in Cohort 2 have had the opportunity to be followed for approximately 8 months from the first dose of enfortumab vedotin, and 4) when all patients in Cohort 2 have had the opportunity to be followed for approximately 8 months from the first dose of enfortumab vedotin.

For illustration purposes, Table 10 below is the summary of expected 95% CIs for Cohort 2 at various analysis timepoints, assuming a 30% observed ORR.

TABLE 10

| Number | Expected 95% |
|---|---|
| of | Confidence |
| Patients | Interval |
| N = 20 | 12%-54% |
| N = 50 | 18%-45% |
| N = 70 | 20%-42% |
| N = 100 | 21%-40% |

Slit lamp examinations will be conducted on at least the first 60 enrolled patients (from Cohorts 1 and/or 2) on Cycle 2 Day 22 (±1 week) and Cycle 6 Day 22 (±1 week). If corneal AEs are observed in <15% of the first 60 enrolled patients and if the events are generally low grade or asymptomatic, the IDMC may make a recommendation to cease Cycle 2 Day 22 and/or Cycle 6 Day 22 slit lamp exams for the remaining patients if warranted based on review of the cumulative ocular safety data. Based on the results from the Phase 1 study (Study ASG-22CE-13-2), as of the data cut-off date of 14 Nov. 2016, there were 3 patients who reported experiencing corneal adverse events out of 33 patients on 1.25 kg/mg of enfortumab vedotin. Assuming an event rate of 9% (3/33), with a sample size of 60, the probability of observing >3 events (5%) is 80.0% and the probability of observing <9 events (15%) is 91.2%. If the event rate is higher, the probability of observing <9 events is decreased. For example, if the event rate is 20%, the probability of observing <9 events is decreased to 12.7%. Based on the analysis described above and the review of cumulative ocular safety data, the IDMC recommended that the Cycle 2 Day 22 and Cycle 6 Day 22 slit lamp exams be discontinued in July 2018 and August 2019, respectively.

Sample size calculations were performed using EAST®, Version 6.0, by Cytel Inc.

6.1.8.2 Study Endpoint Definitions

(i) Objective Response Rate

The primary endpoint of this study is the confirmed ORR per IRF. The ORR is defined as the proportion of patients with confirmed CR or PR according to RECIST Version 1.1 (see Table 28) (Eisenhauer et al., Eur J Cancer (2009); 45(2): 228-47.). Patients who do not have at least 2 (initial response and confirmation scan) post-baseline response assessments as described in Section 6.1.7.2 of the protocol will be counted as non-responders.

In addition, ORR per investigator will be analyzed as a secondary endpoint.

(ii) Duration of Response

The DOR is defined as the time from first documentation of objective response (CR or PR that is subsequently confirmed) to the first documentation of PD (per RECIST Version 1.1) or to death due to any cause, whichever comes first.

DOR data will be censored as described below:
Patients who do not have PD and are still on study at the time of an analysis will be censored at the date of the last disease assessment documenting absence of PD;
Patients who have started an antitumor treatment other than the study treatment (with the exception of palliative radiotherapy as described in Section 6.1.6.3(ii)) prior to documentation of PD will be censored at the date of the last disease assessment prior to start of new therapy;
Patients who are removed from the study prior to documentation of PD will be censored at the date of the last disease assessment documenting absence of PD.

DOR will only be calculated for the patients achieving a confirmed CR or PR.

(iii) Disease Control Rate at Week 16

DCR16 per IRF is defined as the proportion of patients with CR, PR, or SD at Week 16 visit, based on IRF assessment. Responses do not need to be confirmed to be scored as responders for the purpose of determining DCR16. Patients whose disease response cannot be assessed as CR, PR, or SD at Week 16 or later will be scored as non-responders for calculating the DCR. An exception is patients who have a CR, PR, or SD subsequent to Week 16, but who miss Week 16; these patients will be counted as having disease control at Week 16.

In addition, DCR16 per investigator will be also summarized.

(iv) Progression-Free Survival

PFS is defined as the time from start of study treatment to first documentation of objective tumor progression (PD per RECIST Version 1.1), or to death due to any cause, whichever comes first.

The same censoring rules outlined in Section 6.1.8.2 for DOR will be applied to PFS. Patients lacking an evaluation of tumor response after their first dose will have their event time censored at Day 1.

(v) Overall Survival

OS is defined as the time from start of study treatment to date of death due to any cause. In the absence of death, OS will be censored at the last date the patient is known to be alive.

6.1.8.3 Statistical and Analytical Plans

The statistical and analytical plans presented below summarize the more complete plans to be detailed in the statistical analysis plan (SAP). A change to the data analysis methods described in the protocol will require a protocol amendment only if it alters a principal feature of the protocol. The SAP will be finalized prior to database lock. Any changes to the methods described in the final SAP will be described and justified in the clinical study report.

(i) General Considerations

In general, descriptive statistics will be presented that include the number of observations, mean, median, standard deviation, minimum and maximum for continuous variables, and the number and percentages (of non-missing) per category for categorical variables.

Unless otherwise specified, CIs will be calculated at 2-sided 95% level.

The 2-sided 95% exact CI using Clopper-Pearson method (Clopper et al., Biometrika (1934); 26(4): 404-413) will be calculated for the response rates where applicable (e.g., ORR).

For time-to-event endpoints, the median survival time will be estimated using the Kaplan-Meier method; the associated 95% C1 will be calculated based on the complementary log-log transformation (Collett et al., Chapman & Hall (1994): 237-251).

Unless otherwise specified, summaries and analyses will be provided by cohort (i.e., platinum-treated and platinum-naïve and cisplatin-ineligible). Summaries and analyses may also be provided overall.

Data Transformations and Derivations

Time variables based on two dates, e.g., Start Date and End Date, will be calculated as (End Date−Start Date+1) (in days) unless otherwise specified in the planned analysis section.

Unless otherwise specified, baseline values used in all analyses will be the most recent non-missing measurement prior to the first dose of study drug.

Analysis Sets

The full analysis set will include all patients who are enrolled and receive any amount of enfortumab vedotin in the study. A patient is considered enrolled if he/she has met all criteria for participation in the study and has the sponsor's approval as documented in the eCRF. The full analysis set will be used as the primary dataset for efficacy analysis. Patient demographics and baseline disease characteristics will be summarized based on the full analysis set.

The safety analysis set will include all patients who receive any amount of enfortumab vedotin. The safety analysis set will be used for all safety analyses.

The efficacy evaluable set will include all patients in the full analysis set who started treatment with enfortumab vedotin at least 8 months before the analysis data cutoff. The efficacy evaluable set will be used for the additional analyses of efficacy endpoints at the time of the interim analyses of Cohort 2 to allow adequate follow up for a stable estimate of ORR and DOR

The PK analysis set will include all patients who received enfortumab vedotin and from whom at least one blood sample was collected and assayed for enfortumab vedotin, MMAE, or TAb concentration. Corresponding records of the time of dosing and sample collection must also be available for all enfortumab vedotin, MMAE, and TAb concentration data. The PK analysis set will be used for PK analyses.

Additional analysis sets of patients may be defined in the SAP.

Examination of Subgroups

As exploratory analyses, subgroup analyses may be conducted for selected endpoints. Subgroups may include but are not limited to the following:
Number of Bellmunt risk factors (ECOG performance status >0; hemoglobin level
<10 g/dL; presence of liver metastases) (Bellmunt et al., Clin Oncol (2010); 28(11); 1850-5)
Number of prior systemic therapies
Best response to prior CPI therapy
Liver metastasis at baseline

Timing of Analyses

The primary analysis on Cohort 1 (platinum-treated patients) will be conducted when enrollment is completed in Cohort 1, and all patients in the cohort have been followed for at least 6 months, or have discontinued from study, or had 30 days safety follow-up after PD, whichever comes first. Analysis for Cohort 2 will occur at 4 timepoints: 1) at the time of analysis of Cohort 1, 2) when approximately 50 patients in Cohort 2 have had the opportunity to be followed for approximately 8 months from the first dose of enfortumab vedotin, 3) when approximately 70 patients in Cohort 2 have had the opportunity to be followed for approximately 8 months from the first dose of enfortumab vedotin, and 4) when all patients in Cohort 2 have had the opportunity to be followed for approximately 8 months from the first dose of enfortumab vedotin. All patients may also be analyzed at the time of analysis of Cohort 1 and/or Cohort 2.

Additional cutoff dates may be defined and corresponding database locks may occur to allow for more precise estimates of time-to-event endpoints.

(ii) Patient Disposition

An accounting of study patients by disposition will be tabulated and the number of patients in each analysis set will be summarized. Patients who discontinue study treatment and patients who withdraw from the study will be summarized with reason for discontinuation or withdrawal using the full analysis set.

(iii) Patient Characteristics

Demographics and other baseline characteristics will be summarized using the full analysis set. Details will be provided in the SAP.

(iv) Treatment Administration

Treatment administration will be summarized for safety analysis set. Summary statistics for duration of therapy (weeks) and the number of cycles per patient will be presented, as well as the number and percentage of patients who were treated at each cycle and completed each cycle. Details will be provided in the SAP.

(v) Efficacy Analyses

The primary analysis of efficacy endpoints will be based on the full analysis set. At the time of the interim analysis for Cohort 2, the efficacy evaluable set will be used for the additional analyses of efficacy endpoints to allow adequate follow up for a stable estimate of ORR and DOR.

(a) Primary Efficacy Analyses

The primary endpoint of this study is the confirmed ORR per IRF. The ORR is defined as the proportion of patients with confirmed CR or PR according to RECIST Version 1.1 (see Table 28) (Eisenhauer et al., Eur J Cancer (2009); 45(2): 228-47.). Patients who do not have at least 2 (initial response and confirmation scan) post-baseline response assessments as described in Section 6.1.7.2 of the protocol will be counted as non-responders.

The ORR per IRF and its exact 2-sided 95% C1 using the Clopper-Pearson method (Clopper et al., Biometrika (1934): 26(4): 404-413) will be calculated.

There are 2 cohorts of CPI-treated patients in the study: Cohort 1) platinum-treated patients, and Cohort 2) platinum-naïve and cisplatin-ineligible patients. The primary endpoint will be analyzed separately for each cohort and may be analyzed for all patients, combining both Cohorts 1 and 2.

(b) Secondary Efficacy Analyses

The analyses on secondary endpoints, confirmed ORR per investigator, DCR16 per IRF, and DCR₁₆ per investigator assessment will be summarized, and their exact 2-sided 95% CIs using the Clopper-Pearson method (Clopper et al., Biometrika (1934); 26(4): 404-413) will be calculated.

Secondary endpoints, such as DOR per IRF. DOR per investigator, PFS per IRF. PFS per investigator, and OS, are time-to-event endpoints, and they will be analyzed using Kaplan-Meier methodology and Kaplan-Meier plots will be provided. Details on the censoring algorithm will be provided in the SAP.

(vi) Pharmacokinetic and ATA Analyses

Plasma enfortumab vedotin ADC, TAb, and MMAE concentrations will be summarized with descriptive statistics at each PK sampling timepoint using the PK analysis set. These data may be combined with data from previous studies for population PK and PK/pharmacodynamics analyses. The relationship between enfortumab vedotin PK and pharmacodynamics endpoints, safety, or efficacy may be explored.

The incidence of ATA will be summarized by visit and overall using the safety analysis set.

(vii) Quality of Life Analyses

The PRO based on EQ-5D, and QLQ-C30 will be summarized over time with descriptive statistics by visit, using the full analysis set.

(viii) Biomarker Analyses

Relationships of biomarker parameters (e.g., pre-treatment values, absolute and relative changes from pre-treatment) to efficacy, safety, and pharmacokinetic parameters will be explored. Relationships and associated data that are determined to be of interest will be summarized. Details of these analyses will be described separately.

(ix) Safety Analyses

The safety analysis set will be used to summarize all safety endpoints.

(a) Extent of Exposure

Duration of treatment, number of cycles, total dose and dose intensity will be summarized. Dose modifications will also be summarized. Details will be provided in the SAP.

(b) Adverse Events

An overview of AEs will provide a tabulation of the incidence of all AEs, treatment-emergent AEs, treatment-related AEs, Grade 3 and higher AEs. SAEs, treatment-related SAEs, deaths, and AEs leading to study treatment discontinuation. Adverse events will be defined as treatment emergent if they are newly occurring or worsened following study treatment.

AEs will be classified by system organ class and preferred tem using the Medical Dictionary for Regulatory Activities (MedDRA) and graded using NCI CTCAE, Version 4.03.

The incidence of AEs will be summarized by system organ class, preferred term, severity and relationship to study drug. In the event of multiple occurrences of the same AE in one patient, the AE will be counted once as the occurrence for the maximum grade. AEs leading to premature discontinuation of study drug will be summarized and listed in the same manner.

All AEs will be listed.

(c) Deaths and Serious Adverse Events

SAEs will be listed and summarized in the same manner as all AEs. Events with a fatal outcome will be listed.

(d) Clinical Laboratory Results

Summary statistics for lab values and for change from baseline will be tabulated as appropriate by scheduled visit. Laboratory values will be listed with grade per NCI CTCAE v4.03 and flagged when values are outside the normal reference range.

(e) Other Safety Analyses

ECOG Status

ECOG status will be summarized for each visit. Shifts from baseline to the best and worst postbaseline score may be tabulated.

ECG

ECG status (normal, abnormal clinically significant, or abnormal not clinically significant) may be summarized for each scheduled and unscheduled ECG, and shifts from baseline may be tabulated.

(x) Interim Analyses

An IDMC will periodically monitor the trial for safety. The IDMC will review expedited SAEs as they are received. Further details will be provided in the IDMC Charter.

In addition, an ongoing real-time review of SAEs will be conducted by the sponsor's Pharmacovigilance.

Additionally, available data for Cohort 2 will be summarized at the time of the primary analysis of Cohort 1 (e.g., the estimated ORR and its 2-sided 95% exact Clopper-Pearson CI), after approximately 50 patients in Cohort 2 have had the opportunity to be followed for approximately 8 months from the first dose of enfortumab vedotin, and after approximately 70 patients in Cohort 2 have had the opportunity to be followed for approximately 8 months from the first dose of enfortumab vedotin. All patients may also be analyzed at the time of analysis of Cohort 1 and/or Cohort 2.

### 6.1.9 Study Results

Cohort 2 completed enrollment February 2020 with 91 subjects enrolled and 89 treated. The results for cohort 2 patients who underwent the treatment methods as provided in this Section (Section 6) are described below. The results from cohort 1 patients (which completed enrollment July 2018 with 128 subjects enrolled and 125 treated) are included as comparison for various analyses below as indicated.

Regarding disposition, as shown in Table 78, progression was the most common reason for discontinuation, with 16 subjects remained on treatment.

TABLE 11

| | Cohort 2 | |
|---|---|---|
| | (N = 91) | |
| | n (%) | |
| Number of subjects received any | 89 | (97.8) |
| amount of enfortumab vedotin | | |
| Number of subjects on treatment | 16/89 | (18.0) |
| Number of subjects off treatment | 73/89 | (82.0) |

Reason for treatment discontinuation

| | | |
|---|---|---|
| Progressive disease | 45/89 | (50.6) |
| Adverse event | 21/89 | (23.6) |
| Physician decision | 1/89 | (1.1) |
| Subject decision | 6/89 | (6.7) |
| Number of subjects off study | 49 | (53.8) |

Reason for study discontinuation

| Withdrawal by subject | 3 | (3.3) |
|---|---|---|
| Lost to follow-up | 0 | |
| Death | 44 | (48.4) |
| Other | 2 | (2.2) |

Regarding demographics, as shown in Table 12, majority of the participants were males with a median age 75 years old. About one third of the participants were from Europe or Asia, and most of them with an ECOG performance status of 1 to 2, 70% of the participants had moderate or severe renal insufficiency. It is worth noting that renal insufficiency was the most common reason for cisplatin ineligibility see 6.1.14.

TABLE 12

Cohort 2
(N = 89)

Age (yr)

| | |
|---|---|
| Median | 75.0 |
| Min, Max | 49, 90 |

Sex, n (%)

| | |
|---|---|
| Male | 66 (74.2) |
| Female | 23 (25.8) |

Race, n (%)

| | |
|---|---|
| Asian | 20 (22.5) |
| White | 62 (69.7) |
| Not Reportable | 7 (7.9) |

Geographic region, n (%)

| | |
|---|---|
| North America | 57 (64.0) |
| Europe | 14 (15.7) |
| Asia | 18 (20.2) |

ECOG performance status, n (%)

| | |
|---|---|
| 0 | 37 (41.6) |
| 1 | 41 (46.1) |
| 2 | 11 (12.4) |

Body mass index (kg/m²)

| | |
|---|---|
| Median | 25.3 |
| Min, Max | 18, 39 |
| >=30 kg/m² | 13 (14.6) |

Renal function based on creatinine clearance, n (%)

| | |
|---|---|
| Normal: >=90 mL/min | 5 (5.6) |
| Mild decrease: >=60 and <90 mL/min | 22 (24.7) |
| Moderate decrease: >=30 and <60 mL/min | 60 (67.4) |
| Severe decrease: >=15 and <30 mL/min | 2 (2.2) |

Regarding the baseline disease characteristics, as shown in Table 13, when comparing with cohort 1, cohort 2 had shorter time from diagnosis with metastatic disease. Furthermore, primary tumor in upper tract and lymph node only disease were more common; 79% of cohort 2 had visceral metastases including 24% with liver metastases.

TABLE 13

| | |
|---|---|
| | Cohort 2 |
| Disease Characteristics | (N = 89) |

Current extent of disease, n (%)

| | |
|---|---|
| Locally advanced | 1 (1.1) |
| Metastatic | 88 (98.9) |
| Time from diagnosis of metastatic disease | 9.1 |
| to enrollment (mo.), median | |
| Range | 1, 44 |

Primary tumor location, n (%)

| | |
|---|---|
| Upper tract | 38 (42.7) |
| Bladder/other | 51 (57.3) |

Metastasis sites at baseline^{a}, n (%)

| | |
|---|---|
| Lymph nodes only | 18 (20.2) |
| Visceral disease | 70 (78.7) |
| Bone | 22 (24.7) |
| Liver | 21 (23.6) |
| Lung | 41 (46.1) |

Number of Bellmunt risk factors, n (%)

| | |
|---|---|
| 0 | 26 (29.2) |
| 1 | 40 (44.9) |
| 2 | 18 (20.2) |
| 3 | 5 (5.6) |

Regarding prior therapies, as shown in Table 14, almost all of the participates received PD-1 or PD-L1 (abbreviated as “PD-(L)1” or “PD-1/PD-L1”) inhibitor in first line therapy, and majority of them discontinued PD-(L) with less or equal to 3 months prior to the enrollment. The proportion who responded to prior PD-(L)1 was consistent with published data, 40% had prior nephrectomy.

TABLE 14

| | Cohort 2 |
|---|---|
| | (N = 89) |
| Number of prior systemic therapies in | 1 (1, 4) |
| metastatic setting^{a,} median (range) | |

First line therapy received, n (%)

| Platinum-based | 1 (1.1) |
|---|---|
| PD(L)1 monotherapy | 63 (70.8) |
| PD(L)1 + platinum | 10 |
| PD(L)1 + other agent | 24 (27.0) |
| Other | 1 (1.1) |
| Time from completion/discontinuation of most recent | |
| PD(L)1 containing therapy to 1st study dose (months) | |
| <= 3 Months | 71 (79.8) |
| >3 Months | 18 (20.2) |

Best Response to PD(L)1 containing therapy, n (%)

| | |
|---|---|
| Complete Response | 2 (2.2) |
| Partial Response | 20 (22.5) |
| Stable Disease | 30 (33.7) |
| Progressive Disease | 37 (41.6) |
| Surgery | |
| Prior nephrectomy, n (%) | 35 (39.3) |

^{a}Includes prior systemic therapies in the locally advanced or metastatic setting, or PD(L)1 containing therapy in the neoadjuvant/adjuvant setting and the subject progressed within 3 months of therapy completion, or platinum-based therapy in the neoadjuvant/adjuvant setting and the subject progressed within 12 months of therapy completion.

According to the blinded independent central review, ORR was 51.7% with CR as 20.2%, as shown in Table 15.

TABLE 15

| | Cohort 2 |
|---|---|
| | (N = 89) |
| Best Overall Responseª, n (%) | |
| Complete Response (CR) | 18 (20.2) |
| Partial Response (PR) | 28 (31.5) |
| Stable Disease (SD) | 27 (30.3) |
| Progressive Disease (PD) | 8 (9.0) |
| Not Evaluable (NE)C | 8 (9.0) |
| ORR (CR or PR), n (%) | 46 (51.7) |
| 95% CI^{b} for ORR | (40.8, 62.4) |

^{a}Best overall response according to RECIST v1.1. CR or PR were confirmed with repeat scans more than or equal to 28 days after initial response.
^{b}Computed using the Clopper-Pearson method (Clopper et al., Biometrika (1934); 26(4): 404-413)
^{c}Includes 5 subjects who did not have response assessment post-baseline, 2 subjects whose post-baseline assessment did not meet the minimum interval requirement for stable disease, and 1 subject whose response cannot be assessed due to incomplete anatomy.

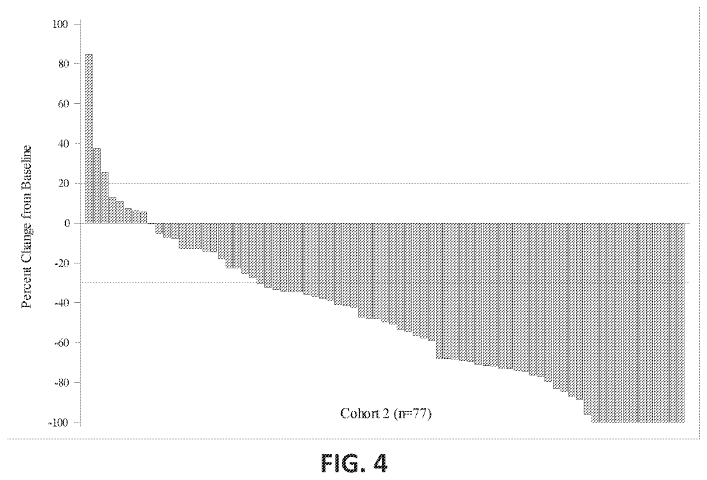

Significant tumor reduction was observed, with 88% of the participants had reduction in tumor measurements by blinded independent central review (see FIG. 4).

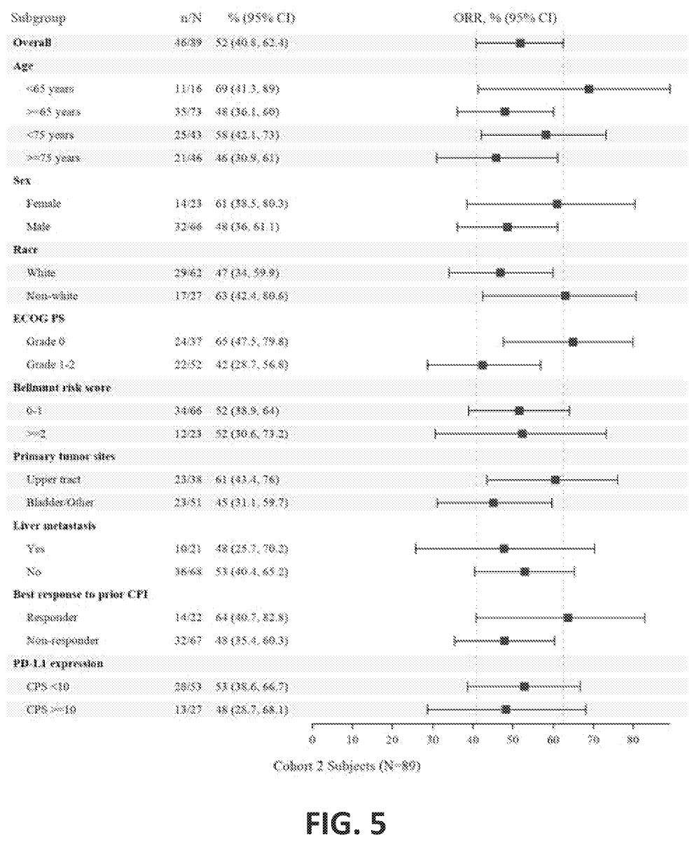

In the ORR subgroup analysis shown in FIG. 5, response rates in all subgroups were similar to that of the overall population. In addition. ORR point estimates more than 40% for all subgroups.

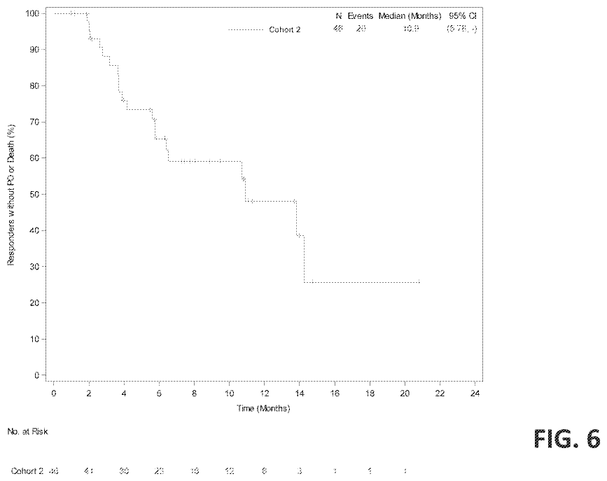

According to the blinded independent central review, median DOR was 10.9 months (95% CI 5.78, -) (see FIG. 6).

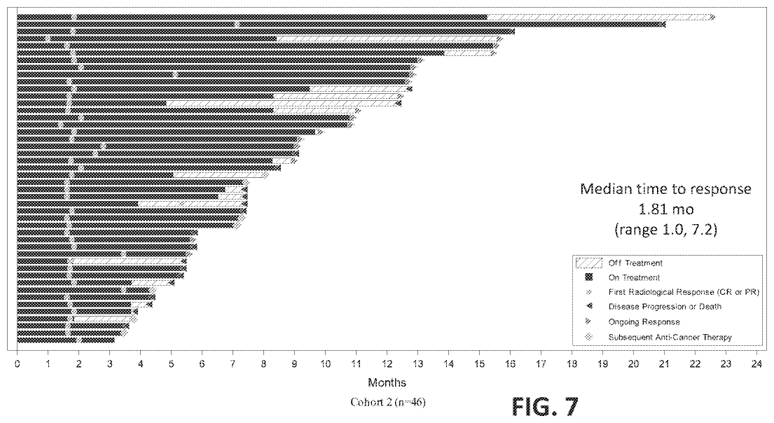

According to the blinded independent central review, most responses were observed early, and 18 out of 46 (39%) with ongoing response (see FIG. 7).

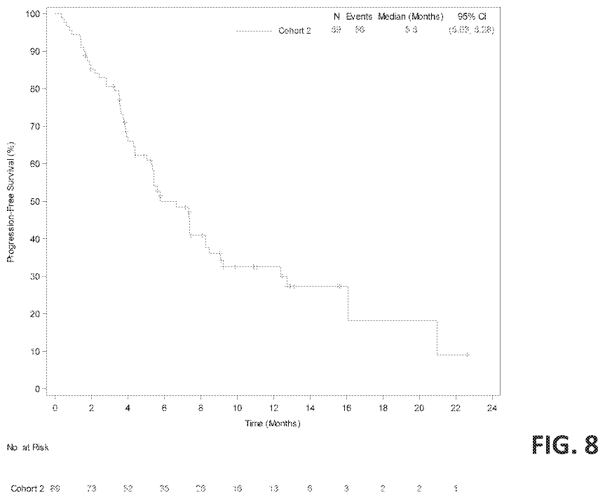

According to the blinded independent central review, median PFS was 5.8 months (95% CI 5.03, 8.28) (see FIG. 8).

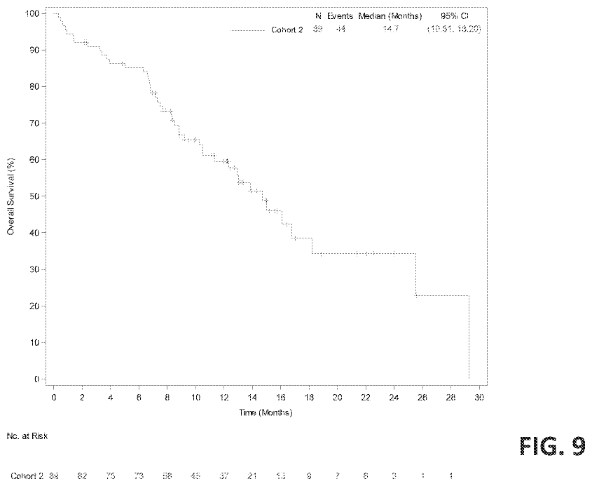

According to the blinded independent central review at this stage of the study, median OS was 14.7 months (95% CI 10.51, 18.20) with a median follow-up of 13.4 months and a range from 0.3 to 29.3 (see FIG. 9).

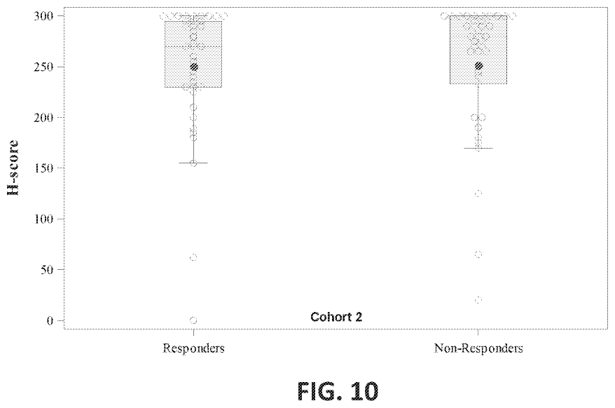

Nectin-4 broadly expressed in bladder cancer. According to the blinded independent central review, Nectin-4 distribution was similar between responders and non-responders (see both FIG. 10 and Table 16)

TABLE 16

| | N | MEAN | STD | MIN | MAX | MEDIAN | Q1 | Q3 |
|---|---|---|---|---|---|---|---|---|
| Responders | 41 | 250.2 | 64.5 | 0 | 300 | 270 | 230 | 295 |
| Non- | 39 | 251.4 | 67 | 20 | 300 | 280 | 233 | 300 |
| Responders | | | | | | | | |

Using duration of treatment as a parameter for summary of exposure, as shown in Table 17, median treatment duration was 6 months, 14% of participants were on treatment for more than or equal to 1 year with maximum of 2 years. The relative dose intensity was 79%.

TABLE 17

Cohort 2
(N = 89)

Duration of Treatment ª (months)

| | |
|---|---|
| Median | 5.98 |
| Min, Max | 0.3, 24.6 |

Number of cycles ^{b} per subject

| | |
|---|---|
| Median | 6.0 |
| Min, Max | 1, 24 |
| 1 | 13 (14.6) |
| 2-3 | 14 (15.7) |
| 4-5 | 14 (15.7) |
| 6-11 | 36 (40.4) |
| ≥12 | 12 (13.5) |
| Relative Dose Intensity, % | 79.0 |

^{a}Duration of treatment is the time from the first dose of study drug to the earliest of the following: Day 28 of the last treatment cycle, date of death, start of subsequent anti- cancer therapy, or analysis data cutoff date if the subject is still on treatment at the time of the analysis.
^{b}Cycle with any amount (>0) of enfortumab vedotin received.

Regarding the overall summary of treatment-emergent adverse events, the safety profile was consistent and manageable, as shown in Table 18.

TABLE 18

| | Cohort 1 | Cohort 2 | Total |
|---|---|---|---|
| | (N = 125) | (N = 89) | (N = 214) |
| | n (%) | (n (%) | n (%) |
| Any TEAEs | 125 (100.0) | 89 (100.0) | 214 (100.0) |
| Treatment-related TEAEs | 117 (93.6) | 86 (96.6) | 203 (94.9) |
| Any grade 3-5 TEAEs | 92 (73.6) | 62 (69.7) | 155 (72.4) |
| Treatment-related grade | 70 (56.9) | 49 (55.1) | 119 (55.6) |
| 3-5 TEAEs | | | |
| Any serious TEAEs | 58 (46.4) | 35 (39.3) | 94 (43.9) |
| Treatment-related serious | 24 (19.2) | 15 (16.9) | 39 (18.2) |
| TEAEs | | | |
| Any TEAEs leading to | 21 (16.8) | 18 (20.2) | 38 (18.2) |
| treatment discontinuation | | | |
| Treatment-related TEAEs | 15 (12.0) | 14 (15.7) | 29 (13.6) |
| leading to treatment | | | |
| discontinuation | | | |
| Any TEAEs leading to | 7 (5.6) | 8 (9.0) | 15 (7.0) |
| death | | | |
| Treatment-related | 0 | 3 (3.4) | 3 (1.4) |
| TEAEs leading to death* | | | |

*1 additional treatment related death in cohort 2 due to pneumonitis, and one additional treatment related death in cohort 1 due to interstitial lung disease

Most common treatment-related TEAEs (all grades) in cohort 2 were similar to Cohort 1, which include alopecia, fatigue, and peripheral sensory neuropathy. Relative to cohort 1, cohort 2 showed less fatigue, less decreased appetite, less nausea, and less diarrhea, while numerically more peripheral sensory neuropathy and rash maculo-papular, as shown in Table 19.

TABLE 19

| | Cohort 1 | Cohort 2 | Total |
|---|---|---|---|
| | (N = 125) | (N = 89) | (N = 214) |
| Preferred Term | n (%) | n (%) | n (%) |
| Overall | 117 (93.6) | 86 (96.6) | 203 (94.9) |
| Alopecia | 62 (49.6) | 45 (50.6) | 107 (50.0) |
| Fatigue | 62 (49.6) | 30 (33.7) | 92 (43.0) |
| Peripheral sensory neuropathy | 50 (40.0) | 42 (47.2) | 92 (43.0) |
| Decreased appetite | 55 (44.0) | 29 (32.6) | 84 (39.3) |
| Dysgeusia | 47 (37.6) | 24 (27.0) | 71 (33.2) |
| Nausea | 50 (40.0) | 20 (22.5) | 70 (32.7) |
| Diarrhoea | 41 (32.8) | 20 (22.5) | 61 (28.5) |
| Pruritus | 32 (25.6) | 27 (30.3) | 59 (27.6) |
| Rash maculo-papular | 28 (22.4) | 27 (30.3) | 55 (25.7) |
| Weight decreased | 29 (23.2) | 23 (25.8) | 52 (24.3) |
| Anaemia | 27 (21.6) | 22 (24.7) | 49 (22.9) |
| Dry skin | 30 (24.0) | 16 (18.0) | 46 (21.5) |
| Dry eye | 25 (20.0) | 11 (12.4) | 36 (16.8) |
| Lacrimation increased | 21 (16.8) | 9 (10.1) | 30 (14.0) |
| Vomiting | 19 (15.2) | 7 (7.9) | 26 (12.1) |
| Oedema peripheral | 15 (12.0) | 10 (11.2) | 25 (11.7) |
| Aspartate aminotransferase | 17 (13.6) | 7 (7.9) | 24 (11.2) |
| increased | | | |
| Neutropenia | 13 (10.4) | 11 (12.4) | 24 (11.2) |

Regarding the treatment-related TEAEs with more than or equal to Grade 3 occurring in 5 or more subjects, neutropenia, anemia, fatigue were the most common treatment-related TEAEs with more than or equal to Grade 3 in both cohorts and occurred at a similar frequency. Relative to cohort 1, cohort 2 had numerically less febrile neutropenia, and numerically more acute kidney injury, more rash maculo-papular, and more decreased appetite, as shown in Table 20.

TABLE 20

| | Cohort 1 | Cohort 2 | Total |
|---|---|---|---|
| | (N = 125) | (N = 89) | (N = 214) |
| Preferred Term | n (%) | n (%) | n (%) |
| Overall | 70 (56.0) | 49 (55.1) | 119 (55.6) |
| Neutropenia | 10 (8.0) | 8 (9.0) | 18 (8.4) |
| Anaemia | 9 (7.2) | 5 (5.6) | 14 (6.5) |
| Fatigue | 8 (6.4) | 6 (6.7) | 14 (6.5) |
| Rash maculo-papular | 5 (4.0) | 7 (7.9) | 12 (5.6) |
| Hyperglycaemia | 5 (4.0) | 5 (5.6) | 10 (4.7) |
| Lipase increased | 5 (4.0) | 5 (5.6) | 10 (4.7) |
| Diarrhoea | 3 (2.4) | 5 (5.6) | 8 (3.7) |
| Amylase increased | 3 (2.4) | 3 (3.4) | 6 (2.8) |
| Aspartate aminotransferase | 4 (3.2) | 2 (2.2) | 6 (2.8) |
| increased | | | |
| Decreased appetite | 1 (0.8) | 5 (5.6) | 6 (2.8) |
| Febrile neutropenia | 5 (4.0) | 1 (1.1) | 6 (2.8) |
| Hyponatraemia | 3 (2.4) | 3 (3.4) | 6 (2.8) |
| Neutrophil count decreased | 3 (2.4) | 3 (3.4) | 16 (2.8) |
| Rash erythematous | 4 (3.2) | 2 (2.2) | 6 (2.8) |
| Acute kidney injury | 1 (0.8) | 4 (4.5) | 5 (2.3) |
| Peripheral sensory neuropathy | 2 (1.6) | 3 (3.4) | 5 (2.3) |
| Pruritus | 2 (1.6) | 3 (3.4) | 5 (2.3) |

Among SAEs (unrelated and related) occurring in 5 or more subjects, acute kidney injury was the most common SAE in cohort 2 (10.1%), while urinary tract infection and cellulitis were the most common SAE in in cohort 1 (4.8% each). Among the related SAEs occurring in 5 or more subjects, acute kidney injury was the most common related SAE in cohort 2 (3.4%), while febrile neutropeniamost was the common in cohort 1 (4%), as shown in Table 21.

TABLE 21

| | Cohort 1 | | Cohort 2 | | Total | |
|---|---|---|---|---|---|---|
| | (N = 125) | | (N = 89) | | (N = 214) | |
| | n (%) | | n (%) | | n (%) | |
| Preferred Term | All | Related | All | Related | All | Related |
| Overall | 59 (47.2) | 24 (19.2) | 35 (39.3) | 15 (16.9) | 94 (43.9) | 39 (18.2) |
| Acute kidney | 4 (3.2) | 1 (0.8) | 9 (10.1) | 3 (3.4) | 13 (6.1) | 4 (1.9) |
| injury | | | | | | |
| Urinary tract | 6 (4.8) | — | 3 (3.4) | — | 9 (4.2) | — |
| infection | | | | | | |
| Sepsis | 4 (3.2) | — | 4 (4.5) | — | 8 (3.7) | — |
| Cellulitis | 6 (4.8) | 2 (1.6) | — | — | 6 (2.8) | 2 (0.9) |
| Diarrhoea | 3 (2.4) | 1 (0.8) | 3 (3.4) | 2 (2.2) | 6 (2.8) | 3 (1.4) |
| Febrile | 5 (4.0) | 5 (4.0) | 1 (1.1) | 1 (1.1) | 6 (2.8) | 6 (2.8) |
| neutropenia | | | | | | |
| Pneumonia | 3 (2.4) | — | 3 (3.4) | 1 (1.1) | 6 (2.8) | 1 (0.5) |
| Nausea | 3 (2.4) | 3 (2.4) | 2 (2.2) | 1 (1.1) | 5 (2.3) | 4 (1.9) |

It has been shown that peripheral sensory neuropathy was the most common treatment-related AE leading to dose discontinuation in both cohorts, as shown in Table 22.

TABLE 22

| | Cobort 1 | Cohort 2 | Total |
|---|---|---|---|
| | (N = 125) | (N = 89) | (N = 214) |
| Preferred Term | n (%) | n (%) | n (%) |
| Overall | 15 (12.0) | 14 (15.7) | 29 (13.6) |
| Peripheral sensory | 8 (6.4) | 4 (4.5) | 12 (5.6) |
| neuropathy | | | |
| Acute kidney injury | — | 2 (2.2) | 2 (0.9) |
| Lipase increased | — | 2 (2.2) | 2 (0.9) |
| Asthenia | 1 (0.8) | — | 1 (0.5) |
| Dermatitis bullous | — | 1 (1.1) | 1 (0.5) |
| Drug eruption | 1 (0.8) | — | 1 (0.5) |
| Enterocolitis | 1 (0.8) | — | 1 (0.5) |
| Fatigue | 1 (0.8) | — | 1 (0.5) |
| Hyperglycaemia | 1 (0.8) | — | 1 (0.5) |
| Interstitial lung disease | 1 (0.8) | — | 1 (0.5) |
| Metabolic acidosis | — | 1 (1.1) | 1 (0.5) |
| Multiple organ dysfunction | — | 1 (1.1) | 1 (0.5) |
| syndrome | | | |
| Muscular weakness | — | 1 (1.1) | 1 (0.5) |
| Peripheral motor neuropathy | — | 1 (1.1) | 1 (0.5) |
| Pneumonitis | — | 1 (1.1) | 1 (0.5) |
| Stevens-Johnson syndrome | 1 (0.8) | — | 1 (0.5) |

Adverse events of special interest rates were generally consistent with Cohort 1. Only rash numerically was higher, and ocular events were generally lower, including corneal events (IDMC discontinued ocular exams) (see Table 23).

TABLE 23

| | Cohort 1 | | Cohort 2 | | Total | |
|---|---|---|---|---|---|---|
| | N = 125 | | N = 89 | | N = 214 | |
| | (%) | | (%) | | (%) | |
| | All | | All | | All | |
| | Grade | ≥Gr 3 | Grade | ≥Gr 3 | Grade | ≥Gr 3 |
| | N | N | N | N | N | N |
| Event | (%) | (%) | (%) | (%) | (%) | (%) |
| Peripheral | 70 (56) | 5 (4) | 51 (58.4) | 7 (7.9) | 122 (57.0) | 12 (5.6) |
| Neuropathy | | | | | | |
| Rash | 67 (53.6) | 15 (12.0) | 59 (66.3) | 15 (16.9) | 126 (58.9) | 30 (14.0) |
| Hyperglycemia | 20 (16) | 10 (8) | 16 (18) | 8 (9) | 36 (16.8) | 18 (8.4) |
| Ocular Events | | | | | | |
| Dry Eye | 51 (40.8) | — | 27 (30.3) | — | 78 (36.4) | — |
| Disorders | | | | | | |
| Corneal | 18 (14.4) | — | 5 (5.6) | — | 23 (10.7) | — |
| Disorders | | | | | | |
| Blurred Vision | 21 (16.8) | — | 9 (10.1) | — | 30 (14.0) | — |
| IRR | 8 (6.4) | 1 (0.8) | 6 (6.7) | 1 (1.1) | 14 (6.5) | 2 (0.9) |

Table 24 shows TEAEs leading to death (excluding disease progression).

TABLE 24

| | Cohort 1 | Cohort 2 | Total |
|---|---|---|---|
| Preferred Term | (N = 125) | (N = 89) | (N = 214) |
| Overall | 4 (3.2) | 5 (5.6) | 9 (4.2) |
| Acute kidney injury | — | 2 (2.2) | 2 (0.9) |
| Sepsis | 1 (0.8) | 1 (1.1) | 2 (0.9) |
| Acute respiratory failure | 1 (0.8) | — | 1 (0.5) |
| Cardiac disorder | 1 (0.8) | — | 1 (0.5) |
| Metabolic acidosis | — | 1 (1.1) | 1 (0.5) |
| Multiple organ dysfunction | — | 1 (1.1) | 1 (0.5) |
| syndrome | | | |
| Pneumonia aspiration | 1 (0.8) | — | 1 (0.5) |

1 interstitial lung disease (cohort 1); 1 pneumonitis (cohort 2) both treatment related occurring >30 days after last dose.

Specific information regarding treatment-related deaths in Cohort 2 is shown in Table 25.

TABLE 25

| | | | | | Death | |
|---|---|---|---|---|---|---|
| | | Last Dose | | | <=30 | |
| | First | Date | Death Date | Primary Cause of | Days of | Disease |
| Subject | Dose Date | (Study Day) | (Study Day) | Death | Last Dose | Related |
| 10007- | 15 JUL. 2019 | 22 JUL. 2019 | 29 JUL. 2019 | Metabolic acidosis | Y | N |
| 0180 | | (8) | (15) | | | |
| 10013- | 19 AUG. 2019 | 26 AUG. 2019 | 15 SEP. 2019 | Acute kidney injury | Y | Y |
| 0190 | | (8) | (28) | | | |
| 10030- | 9 SEP. 2019 | 16 SEP. 2019 | 3 OCT. 2019 | Multiple organ | Y | N |
| 0197 | | (8) | (25) | dysfunction syndrome | | |
| 82004- | 11 JUL. 2019 | 11 NOV. 2019 | 29 JAN. 2020 | Pneumonitis | N | N |
| 0178 | | (124) | (203) | | | |

In summary, the safety profile was manageable in patients who had received prior PD-1/PD-L1 therapy and were cisplatin ineligible. The safety profile was manageable and tolerable, with longer duration of treatment supporting the tolerability of this regimen in this cisplatin ineligible population when compared to cohort 1. The profile was consistent with cohort 1 without new safety signals identified. Four deaths assessed as treatment-related by investigators occurred in patients who were 75 years old or older and who had significant comorbidities and poor prognostic factors, which is higher than the one death reported in cohort 1 (n=1). However, cohort 2 was a frail population that was older with increased moderate-severe renal impairment and higher ECOG scores.

Summary of Clinical Outcomes

Taken together, efficacy measures in cohort 2 exceeded historical benchmarks, which supports enfortumab vedotin as platinum-free option in locally advanced or metastatic urothelial cancer (la/mUC) patients who have been treated with a PD1 or PD-L1 therapy and are cisplatin-ineligible.

ORR exceeded that of available therapies for this population, illustrated by the findings that more than half of subjects responded with one fifth achieving complete response. Specifically, ORR was 51.7% (95% CI: 40.8, 62.4), and complete response rate was 20.2% per blinded independent central review. Median duration of response (DOR) was 10.9 months (95% CI 5.78, -). Similar efficacy was observed across all subgroups, progression-free survival and overall survival estimates were encouraging. Subjects received treatment for a median duration of 6 months with a maximum of 24 months. As of the data cutoff date on Sep. 8, 2020, median follow-up was 13.4 months.

The safety profile is manageable in the patient population represented in cohort 2. Most common treatment-related TEAEs in cohort 2 were similar to Cohort 1, which include alopecia, peripheral sensory neuropathy, and fatigue. 55.1% of subjects experienced a treatment-related TEAE greater than or equal to Grade 3, with the most common ones including neutropenia (9%), rash maculo-popular (7.9%), fatigue (6.7%). Discontinuation rate due to treatment-related AEs was 15.7%, among which peripheral neuropathy was the most common reason (4.5%). Four deaths were assessed as treatment-related by investigators occurred in patients who were older than or equal to 75 years old with significant comorbidities and poor prognostic factors. In cohort 1, most common treatment-related adverse events included fatigue, alopecia, decreased appetite, rash and peripheral neuropathy, which were generally mild to moderate. 54% of subjects experienced a Grade 3 or Grade 4 treatment-related TEAE, with the most common ones including neutropenia (8%), anemia (7%), and fatigue (6%). Discontinuation rate due to treatment-related AEs was 12%, and peripheral neuropathy was the most common reason (6%). One case of Stevens-Johnson Syndrome reported. One treatment-related death of interstitial lung disease was confounded by infection. Events in both cohorts and occurred at a similar frequency. Adverse events of interest were also consistent with those previously reported for enfortumab vedotin, and included rash, peripheral neuropathy, and hyperglycemia. Acute kidney injury was the most frequent SAE, occurring at a higher rate than in Cohort 1. It is believed that cisplatin-ineligible population was at higher risk for acute kidney injury due to underlying renal dysfunction. Therefore, the collective safety results are reflective of a manageable safety profile in patients who had received prior PD-1/PD-L1 therapy and were cisplatin ineligible. Notably, subjects in Cohort 2 were representative of a typical cisplatin-ineligible population who have progressed on/after PD-(L)1 inhibitors in 1 L.

Taken together, the results above demonstrate a positive benefit-risk profile in cisplatin-ineligible patients who received prior PD-1/PD-L1 therapy. First, enfortumab vedotin was highly effective, ORR and DOR estimates exceeded those for Cohort 1 and the EV301 clinical trial disclosed previously (see e.g. businesswire.com/news/home/20200918005101/en/, press release dated Sep. 18, 2020). Second, safety was manageable and tolerable in a predominantly elderly and frail population.

In conclusion, based on the robust efficacy and manageable safety profile in cisplatin-ineligible patients with advanced urothelial cancer, who received prior PD-1/PD-L1, the benefit risk profile of enfortumab vedotin is positive.

### 6.1.10 List of Abbreviations and Descriptions of Terms

ADC antibody-drug conjugate
AE adverse event
ALT alanine aminotransferase
AST aspartate aminotransferase
ATA antitherapeutic antibodies
BSC best supportive care
CBC complete blood count
CFR Code of Federal Regulations
CI confidence interval
CNS central nervous system
CPI checkpoint inhibitor
CR complete response
CrCl creatinine clearance
CRF case report form
CT computed tomography
ctDNA Circulating tumor DNA
DCR disease control rate
DCR16 disease control rate at 16 weeks
DLT dose-limiting toxicity
DOR duration of response
ECD extracellular domain
ECG electrocardiogram
ECOG Eastern Cooperative Oncology Group
eCRF electronic case report form
EORTC European Organization for the Research and Treatment of Cancer
EOT end of treatment
EQ-5D EuroQol 5-dimensions
FDA Food and Drug Administration
HbA1c hemoglobin A1c
ICH International Council for Harmonisation
IDMC independent data monitoring committee
IEC independent ethics committee
Ig immunoglobulin
IHC immunohistochemistry
IND investigational new drug
INR international normalized ratio
IRB institutional review board
IRF independent review facility
IRR infusion-related reaction
IV intravenous
LFT liver function test
MMAE monomethyl auristatin E
MRI magnetic resonance imaging
mRNA messenger ribonucleic acid
NCI CTCAE National Cancer Institute's Common Terminology Criteria for Adverse Events
ORR objective response rate
OS overall survival
PBMC peripheral blood mononuclear cell
PCR polymerase chain reaction
PD progressive disease
PD-1 programmed cell death protein 1
PD-L1 programmed death-ligand 1
PFS progression-free survival
PK pharmacokinetics
PP per-protocol
PR partial response
PRO patient reported outcomes
PT prothrombin time
PTT partial thromboplastin time
QLQ-C30 EORTC Quality of Life Questionnaire
QoL quality of life
RECIST Response Evaluation Criteria in Solid Tumors
SAE serious adverse event
SAP statistical analysis plan
SD stable disease
TAb total antibody
TEAE treatment-emergent adverse event
ULN upper limit of normal
vc valine-citrulline

TABLE 26
Schedule of Events

| | | Day | | | | | | | | | | | |
|---|---|---|---|---|---|---|---|---|---|---|---|---|---|
| | | | | | | | | | | | EOT | | |
| | | Screening/ | | | | | | | | | Within | Follow- | |
| | | Baseline | | Enrollment | | | | | | | 30-37 d | up | LTFU |
| | | D −28 | D −7 | Within 7 D | Every 28-day cycle | | | | | | of last | Every 8 | Every 8 |
| | | to 1 | to 1 | of 1st dose | D1 | D3^{A} | D8 | D15 | D17^{A} | D22 | dose^{B} | weeks | weeks |
| | | Visit window | | | | | | | | | | | |
| | | | | | | | | | −24 h/ | | | | |
| | | | | | ±2 d | ±24 h | +2 d | +2 d | +48 h | | | ±7 d | ±7 d |
| Screening/ | Inclusion/exclusion, | X | | Submit | | | | | | | | | |
| baseline | medical history | | | confirmation | | | | | | | | | |
| assessments | Informed consent | X | | of | | | | | | | | | |
| | Acquire and submit tumor | X | | elligibility | | | | | | | | | |
| | specimen^{C} | | | prior to | | | | | | | | | |
| | Complete eye examination^{D} | X | | treatment | | | | | | | | | |
| | Slit lamp eye examination^{D} | | | | | | | | | | X^{O} | | |
| | Brain scan | X | | | | | | | | X^{E} | X^{E} | X^{E} | |
| | Bone scan | X | | | | | | | | X^{E} | X^{E} | X^{E} | |
| | INR/PT/PTT | X | | | | | | | | | | | |
| | Hepatitis B and C screening | X | | | | | | | | | | | |
| | Urinalysis with microscopic | X | | | | | | | | | | | |
| | analysis | | | | | | | | | | | | |
| | HbA1c^{N} | X | | | | | | | | | | | |
| | Pregnancy test (females of | | X | | X | | | | | | X | | |
| | childbearing potential)^{F} | | | | | | | | | | | | |
| Safety | Physical exam | | X | | X^{G} | | | | | | X | X^{H} | |
| assessments | (including weight)^{P} | | | | | | | | | | | | |
| | Height | | X | | | | | | | | | | |
| | Vital signs | | X | | X^{G} | | X | X | | | X | | |
| | CBC with differential^{P} | | X | | X^{G} | | X | X | | | X | | |
| | Chemistry panel^{P} | | X | | X^{G} | | X | X | | | X | | |
| | CrCl | | X | | | | | | | | | | |
| | ECOG performance status^{P} | | X | | X^{G} | | | | | | X | X^{H} | |
| | ECG | | X | | | | | | | | X | | |
| | Concomitant medications | Related to study procedures | | | Collect from Day 1 predose through 30 days | | | | | | | | |
| | Adverse event collection | from time of informed | | | post last study treatment | | | | | | | | |
| | | consent | | | | | | | | | | | |
| PRO/QoL | QLQ-C30 and EQ-5D | | | | X | | | | | | | | |
| Treatment | Study drug administration^{I} | | | | X | | X^{T} | X | | | | | |
| PK/ATA/ | Blood sample collection | See PK, ATA and Biomarker Table (Table 9) for sample collection details | | | | | | | | | | | |
| biomarker | | | | | | | | | | | | | |
| Response | CT scan with contrast of | X | | | | | | | | X^{J,K} | X^{J,L} | X^{H,J} | |
| assessment | chest, abdomen, pelvis | | | | | | | | | | | | |
| | and any other | | | | | | | | | | | | |
| | region of known or | | | | | | | | | | | | |
| | suspected disease^{Q} | | | | | | | | | | | | |
| | Survival status | | | | | | | | | | | | X^{M} |

^{A}Cycles 1 and 2. See PK, ATA and Biomarker Table (Table 9) for sample collection details.
^{B}EOT evaluations must be obtained before the initiation of subsequent anticancer therapy, with the exception of slit lamp examinations. If EOT evaluations are completed before 30 days following the last study treatment, conduct a phone screen 30-37 days following the patient's last study treatment to ensure that no changes in AE profile have occurred.
^{C}Pretreatment tumor tissue (from primary or metastatic site) for biomarker studies must be available for submission to the sponsor prior to study treatment. A minimum of 10 freshly sectioned, unstained charged slides are required. Either archival tissue or pre-treatment fresh tumor tissue (obtained from a fresh biopsy) is acceptable.
^{D}Repeated as clinically indicated throughout the study.
^{E}Repeated at disease assessment timepoints if disease present at baseline, or as clinically indicated throughout the study.
^{F}Either serum or urine pregnancy test. Not required for Cycle 1 if baseline assessment performed within 7 days. Repeat every month (±1 week) for 6 months after EOT. G Not required for Cycle 1 if baseline assessments performed within 1 day.
^{G}Patients who discontinue study treatment for reasons other than objective disease progression by RECIST Version 1. I will continue to have physicale xams (no weight collection is required), ECOG, and response assessments 8 weeks (±1 week) after the previous response assessment scan and every 8 weeks (±1 week) following the previous visit thereafter. After 1 year on study the frequency of follow-up visits and response assessments will be reduced to every 12 weeks (±1 week). The tumor assessments will continue until the patient has radiologically-confirmed disease progression per RECIST as determined by the investigator, initiates a new anticancer therapy, patient death, study closure, or withdrawal of consent, whichever comes first.
^{H}At least 1 week must elapse between doses of enfortumab vedotin.
^{I}Responses will be confirmed with repeat scans at 4 weeks after first documentation of response (±1 week). Following confirmation scans, response assessments should continue with the previous scan schedule (i.e., the schedule should not be adjusted). Tumor imaging should also be performed whenever disease progression is suspected.
^{J}Response assessment will be performed every 8 weeks (±1 week). After 1 year on study, response assessments will be reduced to every 12 weeks (±1 week). The schedule of response assessments should not be adjusted for dose delays/interruptions or other reasons for changes in the timing of a patient's study activities; timepoints for response assessments should be calculated from Cycle 1 Day 1 during treatment.
^{K}Not required if conducted <4 weeks prior to EOT.
^{L}Contact patient for survival status and collection of subsequent anticancer treatment information every 8 weeks (±1 week) after EOT (or 8 weeks from previous protocol visit, whichever is later) until death, study closure, or withdrawal of consent, or patient is lost to follow-up, whichever occurs first. After 1 year on study the frequency of follow-up contacts will be reduced to every 12 weeks
^{M}(±1 week).
^{N}If HbA1c is elevated (26.5%), refer patient to appropriate provider during Cycle 1 for glucose management.
^{O}EOT slit lamp examination required for all patients who experience corneal adverse events during the study and must be performed >4 weeks from last dose.
^{P}May be collected or conducted up to 1 day prior to dosing. Local laboratory results must be reviewed prior to study drug administration in order to determine whether to proceed with dosing or whether dose modification is required.
^{Q}If contrast is contraindicated, see 6.1.13.

TABLE 27
Performance Status Scales Conversion

| Karnofsky | | Lansky | | ECOG | |
|---|---|---|---|---|---|
| Percent | Description | Percent | Description | Score | Description |
| 100 | Normal, no complaints, | 100 | Fully active, normal. | 0 | Normal activity, Fully |
| 90 | no evidence of disease. | 90 | Minor | | active, able to carry on |
| | Able to carry on | | restrictions in | | all pre-disease |
| | normal activity; | | physically | | performance without |
| | minor signs or | | strenuous | | restriction. |
| | symptoms of disease. | | activity. | | |
| 80 | Normal activity with | 80 | Active, but tires more | 1 | Symptoms, but |
| 70 | effort; some signs or | 70 | quickly. | | ambulatory. |
| | symptoms of disease. | | Both greater | | Restricted in physically |
| | Cares for self, unable to | | restriction of, and | | strenuous activity, but |
| | carry on normal activity | | less time spent in, | | ambulatory and able to |
| | or to do active work. | | play activity. | | carry out work of a |
| | | | | | light or sedentary |
| | | | | | nature (e.g., light |
| | | | | | housework, office |
| | | | | | work). |
| 60 | Requires occasional | 60 | Up and around, but | 2 | In bed <50% of the |
| 50 | assistance, but is able to | 50 | minimal active play; | | time. |
| | care for most of his/her | | keeps busy with | | Ambulatory and |
| | needs. | | quieter activities. | | capable of all self-care, |
| | Requires | | Gets dressed, but | | but unable to carry out |
| | considerable | | lies around much | | any work activities. Up |
| | assistance and | | of the day; no | | and about more than |
| | frequent medical | | active play; able | | 50% of waking hours. |
| | care. | | to participate in all | | |
| | | | quiet active play | | |
| | | | and activities. | | |
| 40 | Disabled, requires special | 40 | Mostly in bed, | 3 | In bed >50% of the |
| | care and assistance. | | participates in quiet | | time. |
| | | | activities. | | Capable of only |
| 30 | Severely disabled, | 30 | In bed, needs | | limited self-care, |
| | hospitalization | | assistance even | | confined to bed or |
| | indicated. | | for quiet play. | | chair more than 50% of |
| | Death not imminent. | | | | waking hours. |
| 20 | Very sick, hospitalization | 20 | Often sleeping, play | 4 | 100% bedridden |
| | indicated. | | entirely limited to | | Completely disabled. |
| | Death not imminent. | | very passive | | Cannot carry on any |
| | | | activities. | | self- care. Totally |
| 10 | Moribund, fatal | 10 | No play, does not | | confined to bed or |
| | processes progressing | | get out of bed. | | chair. |
| | rapidly. | | | | |
| 0 | Dead. | 0 | Dead | 5 | Dead |

### 6.1.11 New York Heart Association Classification

A Functional and Therapeutic Classification for Prescription of Physical Activity for Cardiac Patients
Class I: patients with no limitation of activities they suffer no symptoms from ordinary activities.
Class I: patients with slight, mild limitation of activity they are comfortable with rest or with mild exertion.
Class III: patients with marked limitation of activity; they are comfortable only at rest.
Class IV: patients who should be at complete rest, confined to bed or chair any physical activity brings on discomfort and symptoms occur at rest.

On-Line Source: heart.org/HEARTORG/Conditions/HeartFailure/AboutHeartFailure/Classes-of-Heart-Failure_UCM_306328_Article.jsp

TABLE 28
Recist Criteria Summary (Version 1.1)
Response Evaluation Criteria in Solid Tumors

| Term | Definition |
|---|---|
| Complete response (CR) | Disappearance of all target lesions. Any pathological lymph |
| | nodes must have reduction in short axis to <10 mm. |
| Partial response (PR) | A ≥30% decrease in the sum of diameters of target lesions, taking |
| | as reference the baseline sum diameters. |
| Progressive disease (PD) | At least a 20% increase in the sum of diameters of target lesions, |
| | taking as reference the smallest sum on study (this includes the |
| | baseline sum if that is the smallest on study). In addition to the |
| | relative increase of 20%, the sum must also demonstrate an |
| | absolute increase of at least 0.5 cm. The appearance of one or |
| | more new lesions is also considered progression. |
| Stable disease (SD) | Neither sufficient shrinkage to qualify for PR nor sufficient |
| | increase to qualify for PD, taking as reference the smallest sum |
| | diameters while on study. |
| Measurable lesion | Must be accurately measured in at least one dimension (longest |
| | diameter in the plane of measurement is to be recorded) with a |
| | minimum size of 10 mm by CT scan (CT slice thickness no |
| | greater than 5 mm). |
| | A lymph node must be ≥15 mm in short axis when assessed by |
| | CT scan to be considered measurable. |

From RECIST Version 1.1 (Eisenhauer et al., Eur J Cancer (2009); 45(2): 228-47) A response (CR or PR) will be considered confirmed if the following disease assessment (at least 4 weeks after the initial response) still shows response (CR or PR). In cases where the initial response is followed by SD, it will be considered as confirmed if the SD is later followed by PR or CR. For

### 6.1.12 Liver Safety Monitoring and Assessment

The following recommendations are from the Food and Drug Administration (FDA) Guidance for Industry titled “Drug-Induced Liver Injury: Premarketing Clinical Evaluation” issued July 2009.

Any patient with an increase of serum aminotransferases to >3×upper limit of normal (ULN) or bilirubin >2×ULN should undergo detailed testing for liver enzymes (including at least alanine aminotransferase (ALT), aspartate aminotransferase (AST), alkaline phosphatase (ALP), and total bilirubin (TBL)). To confirm the abnormality, testing should be repeated within 72 hours of notification of the test results.

Description of Liver Abnormalities:

Confirmed liver abnormalities will be characterized as Moderate and Severe:

Moderate:
ALT or AST>3×ULN OR Total Bilirubin >2×ULN

Severe:
ALT or AST>3×ULN AND Total Bilirubin >2×ULN (*See description of Hy's Law below)
ALT or AST>8×ULN
ALT or AST>5×ULN for more than 2 weeks
ALT or AST>3×ULN and International Normalized Ratio (INR)>1.5 (if INR testing is applicable/evaluated)
ALT or AST>3×ULN with the appearance of symptoms suggestive of liver injury (e.g., right upper quadrant pain or tenderness) and/or eosinophilia (>5%)

The investigator may determine that abnormal liver function results, other than as described above, may qualify as moderate or severe abnormalities and require additional monitoring and follow-up.

*Hy's Law: Drug-induced jaundice caused by hepatocellular injury, without a significant obstructive component, has a high rate of bad outcomes, from 10 to 50% mortality (or transplant). The 2 “requirements” for Hy's Law are: 1) Evidence that a drug can cause hepatocellular-type injury, generally shown by an increase in transaminase elevations higher 3×ULN (“2×ULN elevations are too common in treated and untreated patients to be discriminating”). 2) Cases of increased bilirubin (at least 2×ULN) with concurrent transaminase elevations at least 3×ULN and no evidence of intra- or extra-hepatic bilirubin obstruction (elevated ALP) or Gilbert's syndrome (Temple et al., Pharmacoepidemiol Drug Saf (2006); 15(4): 241-3.).

Follow-Up Procedures

Confirmed moderate and severe abnormalities in hepatic functions should be thoroughly characterized by obtaining appropriate expert consultations, detailed pertinent history, physical examination and laboratory tests. Patients with confirmed abnormal liver function testing should be followed as described below.

Confirmed moderately abnormal liver function tests (LFTs) should be repeated 2 to 3 times weekly then weekly or less if abnormalities stabilize or the study drug has been discontinued and the patient is asymptomatic.

Severe hepatic liver function abnormalities as defined above, in the absence of another etiology, may be considered an important medical event and may be reported as a SAE. The sponsor should be contacted and informed of all patients for whom severe hepatic liver function abnormalities possibly attributable to study drug are observed.

To further assess abnormal hepatic laboratory finding, it is recommended that the investigator:
Obtain a more detailed history of symptoms and prior or concurrent diseases. Illnesses and conditions such as hypotensive events, and decompensated cardiac disease that may lead to secondary liver abnormalities should be noted. Nonalcoholic steatohepatitis is seen in obese hyperlipoproteinemic and/or diabetic patients, and may be associated with fluctuating aminotransferase levels.
Obtain a history of concomitant drug use (including nonprescription medication, complementary and alternative medications), alcohol use, recreational drug use and special diets
Obtain a history of exposure to environmental chemical agents.
Based on the patient's history, other testing may be appropriate including:
   Acute viral hepatitis (A, B, C, D, E or other infectious agents),
   Ultrasound or other imaging to assess biliary tract disease.
   Other laboratory tests including INR, direct bilirubin.
Consider gastroenterology or hepatology consultations.

Conduct additional testing as determined by the investigator to further evaluate possible etiology. See Section 6.1.6.2(iii)(a) for treatment discontinuation recommendations related to hepatic safety.

### 6.1.13 Scanning and Contrast Guidelines

In decreasing order of preference of the following:

Brain Scan:
1. Brain MRI with gadolinium

If gadolinium is medically contraindicated:
2. Brain MRI without gadolinium
3. Brain CT with IV contrast
4. Brain CT without IV contrast

Chest-Abdomen-Pelvis Scans:
1. Chest-Abdomen-Pelvis CT with IV contrast

If iodine media is medically contraindicated:
2. Chest CT without IV contrast and Abdomen-Pelvis MRI with gadolinium
3. Chest-Abdomen-Pelvis CT without IV contrast (oral contrast is recommended)
4. Chest-Abdomen-Pelvis MRI with gadolinium

CT Oral Contrast
1. Radio opaque agents (e.g., iodine and barium based agents)
2. Radio-lucent agents (whole milk, VoLumen®, water)

### 6.1.14 Renal Insufficiency Was the Most Common Reason for Cisplatin Ineligibility

TABLE 29

| | n (%) |
|---|---|
| Creatinine clearance <60 ml/min | 59 (66.3) |
| ECOG PS of 2 | 6 (6.7) |
| Grade ≥2 hearing loss | 13 (14.6) |
| Creatinine clearance <60 mL/min and ECOG PS of 2 | 3 (3.4) |
| Creatinine clearance <60 mL/min and Grade ≥2 hearing loss | 7 (7.9) |
| ECOG PS of 2 and Grade ≥2 hearing loss | 1 (1.1) |

### 6.2 Example 2—Three-month Follow-up of the Single-Arm, Open-Label, Multicenter Study of Enfortumab Vedotin (ASG-22CE) for Treatment of Patients with Locally Advanced or Metastatic Urothelial Cancer Who Previously Received Immune Checkpoint Inhibitor (CPI) Therapy

6.2.1 Study Methods

As a three-month follow-up study of the study described in Section 6.1 (Example 1), the study methods for this Section 6.2 (Example 2) were as described in Section 6.1 (Example 1). Briefly, as shown and described in Section 6.1 (Example 1), cisplatin (cis)-ineligible, platinum-naïve patients with locally advanced or metastatic urothelial carcinoma (“la/mUC”) who progress on/after PD-1/L1 inhibitors have a poor prognosis and few treatment options. As described herein including in Section 6.1 (Example 1), enfortumab vedotin (EV) is an antibody-drug conjugate directed against Nectin-4, an immunoglobulin-like cell adhesion molecule highly expressed in UC. Section 6.1 (Example 1) describes a pivotal, single-arm, 2-cohort study of EV in la/mUC, in which Cohort 1 focuses on determining the efficacy of EV in adult patients with la/mUC who previously received PD-1/L1 inhibitors and platinum-containing chemotherapy in the neoadjuvant/adjuvant, la/mUC setting. As described in Section 6.1 (Example 1), Cohort 2 of the study focuses on cis-ineligible patients with prior PD-1/L1 inhibitors and no prior platinum for la/mUC. Results of the primary analysis for Cohort 2 are described in Section 6.1 (Example 1). This Section 6.2 (Example 2) describes the analyses with an additional 3 months follow-up of the study described in Section 6.1 (Example 1) to determine the efficacy of EV during the follow-up, including determining the duration of response (DOR). Accordingly, the data presented in this Section 6.2 (Example 2) presents updated data that reflects the additional 3 months follow up relative to the data presented in Section 6.1 (Example 1). As described in Section 6.1 (Example 1), patients in this open-label, multicenter, multinational study received 1.25 mg/kg EV on Days 1, 8, and 15 of each 28-day cycle. Primary endpoint was confirmed objective response rate (ORR) per RECIST 1.1 by blinded independent central review (BICR). Secondary endpoints were DOR, progression-free survival (PFS), overall survival (OS), and safety. At the time of this follow-up study, all responders had been followed for at least 6 months from the onset of response.

6.2.2 Study Results

The three-month follow-up results for Cohort 2 patients who underwent the treatment methods as provided in Section 6.1 are described below. Briefly, 91 patients were enrolled and 89 treated in Cohort 2. Patients were elderly (median age: 75 y [range: 49-90]) with comorbidities, including moderate/severe renal impairment. Patients were cis-ineligible at study entry due to CrCl<60 mL % min (66%), Grade ≥2 hearing loss (15%), or ECOG PS 2 (7%); an additional 12% met≥1 criterion. The primary tumor site was in upper tract in 43% and 79% had visceral metastases, including 24% with liver metastases. Median treatment duration was 6.0 mo (range: 0.3-24.6).

According to the blinded independent central review, confirmed ORR was 51% (95% CI 39.8-61.3), including 22% CR among treated patients, as shown in Table 30.

TABLE 30

| | Cohort 2 |
|---|---|
| | (N = 89) |
| Best Overall Response ª, n (%) | |
| Complete Response (CR) | 20 (22.5) |
| Partial Response (PR) | 25 (28.1) |
| Stable Disease (SD) | 27 (30.3) |
| Progressive Disease (PD) | 19 (10.1) |
| Not Evaluable (NE) ^{c} | 8 (9.0) |
| ORR (CR or PR), n (%) | 45 (50.6) |
| 95% CI ^{b} for ORR | (39.8, 61.3) |

^{a} Best overall response according to RECIST v1.1. CR or PR were confirmed with repeat scans more than or equal to 28 days after initial response.
^{b} Computed using the Clopper-Pearson method (Clopper et al., Biometrika (1934); 26(4): 404-413).
^{c} Includes 5 subjects who did not have response assessment post-baseline, 2 subjects whose post-baseline assessment did not meet the minimum interval requirement for stable disease, and 1 subject whose response cannot be assessed due to incomplete anatomy.

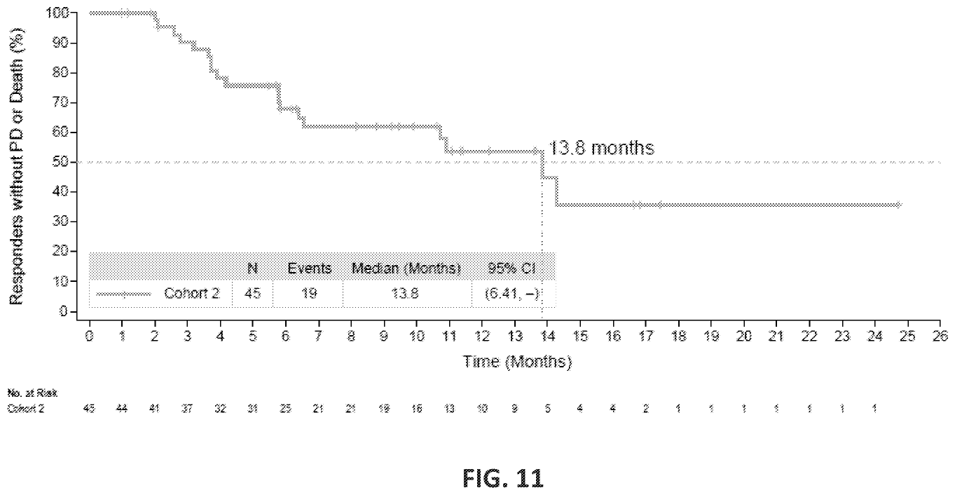

Median DOR at this stage of the study was 13.8 months (95% CI 6.41, -); as shown in Table 31 and FIG. 11.

TABLE 31

| | Cohort 2 |
|---|---|
| | (N = 89) |
| Number of responders (confirmed CR or PR) | 45 |
| Number of responders who progressed or died | 19/45 (42.2) |
| Duration of responseª (months) | |
| Median (95% CI*) | 13.8 (6.41, −) |
| 25^{th}, 75^{th} percentile | 5.8, − |
| Observed min, max | 0.99+. 24.71+ |
| % Responders without PD or death at | |
| 6 months (95% CI^{b}) | 67.9 (51.11, 79,95) |
| 12 months (95% CI^{b}) | 53.7 (35.51, 68.85) |
| 118 months (95% CI^{b}) | 35.8 (14.49, 57.91) |

^{a}As estimated using Kaplan-Meier method.
^{b}Calculated using the complementary log-log transformation method (Collett et al., Chapman & Hall (1994): 237-251).

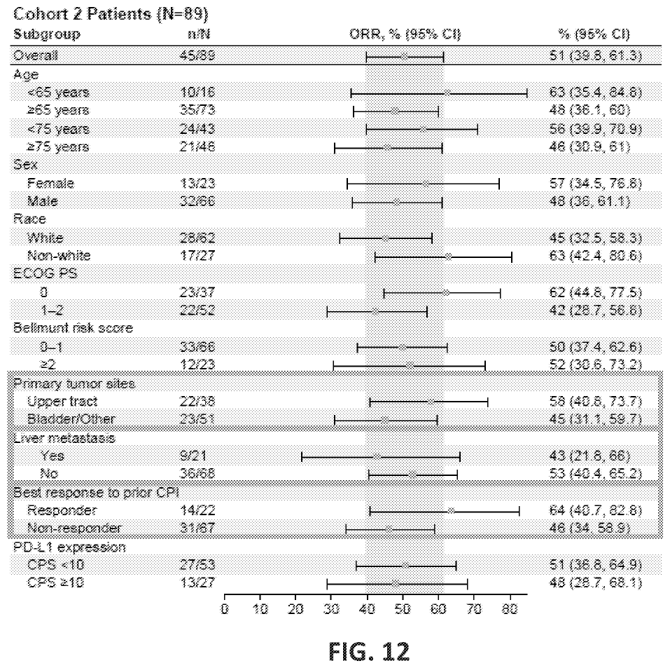

In the ORR subgroup analysis shown in FIG. 12, responses were observed across all subgroups including ORR point estimates of more than 40% for all subgroups. Responses were observed in patients with primary tumor sites in the upper tract (ORR=58%), patients with liver metastasis (ORR=43%), and patients who did not respond to prior PD-1/PD-L1 inhibitors (ORR=46%).

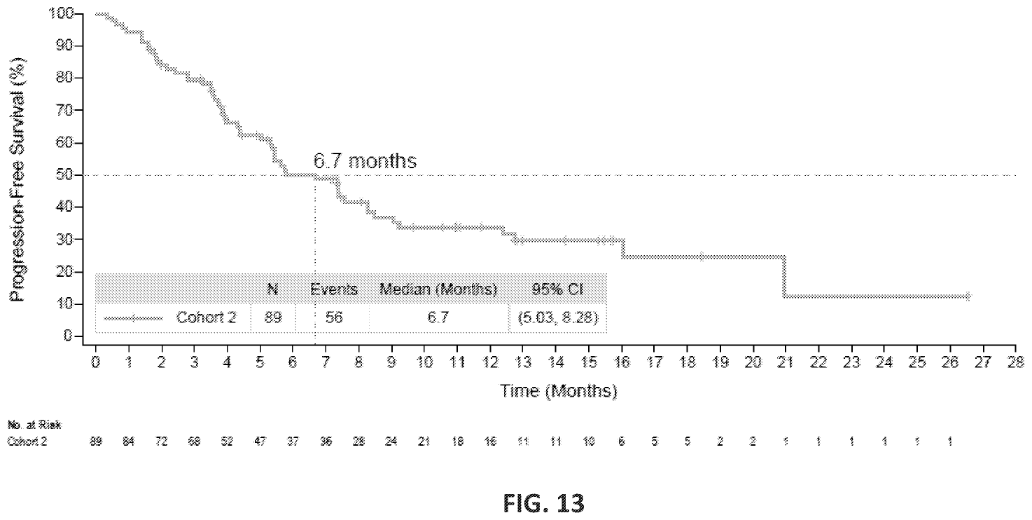

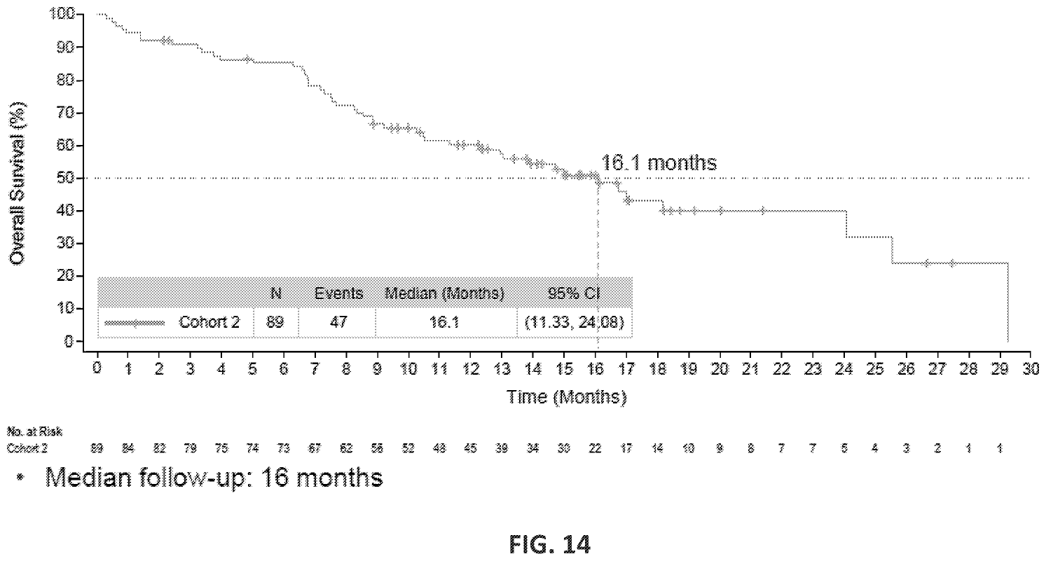

Median PFS was 6.7 months (95% CI 5.0(8.3) (see FIG. 13) and median OS was 16.1 months (95% CI 11.3, 24.1) (see FIG. 14). The median follow-up for overall survival is 16.0 months (95% CI 14.78, 18.20).

Most common treatment-related TEAEs (all-grade) were alopecia (51%), peripheral sensory neuropathy (49%), and fatigue (34%), as shown in Table 32.

TABLE 32

| TRAEs in ≥20% of patients | Patients (N = 89), n (%) | |
|---|---|---|
| (any Grade) or 25% (Grade 3) | Any Grade | ≥Grade 3 |
| Overall TRAEst | 86 (97) | 49 (55) |
| Alopecia | 45 (51) | |
| Peripheral sensory neuropathy | 44 (49) | 3 (3) |
| Fatigue | 30 (34) | 6 (7) |
| Decreased appetite | 29 (33) | 5 (6) |
| Pruritus | 27 (30) | 3 (3) |
| Rash maculo-papular | 27 (30) | 7 (8) |
| Dysgeusia | 25 (28) | |
| nnn | 23 (26) | 1 (1) |
| Weight decreased | | |
| Anemia | 22 (25) | 5 (6) |
| Diarrhea | 20 (22) | 5 (6) |
| Nausea | 20 (22) | 1 (1) |
| Neutropenia | 11 (12) | 8 (9) |
| Hyperglycemia | 8 (9) | 5 (6) |
| Lipase increased | 7 (8) | 5 (6) |

^{a}Treatment-related adverse events based on Preferred Terms

Treatment-related TEAEs of interest included rash (61% all grade, 17%≥G3), peripheral neuropathy (56% all grade, 8%≥G3), and hyperglycemia (10% all grade, 6%≥G3), as shown in Table 33. Adverse events are generally manageable with proper dose modifications and supportive care measures (events categorized based on queries for related MedDRA (Medical Dictionary for Regulatory Activities) terms v. 23.0).

TABLE 33

| | Skin | Peripheral | |
|---|---|---|---|
| | Reactions | Neuropathy | Hyperglycemia |
| Any grade, % | 61 | 56 | 10 |
| >Grade 3. % | 17 | 8 | 6 |
| Median Onset, | 0.5ª | 2.6 | 0.5ª |
| months | | | |
| Resolution/ | 80 | 54 | 89 |
| improvement^{b}, % | | | |

^{a}Most occurred in Cycle 1.
^{b}Resolution/Improvement as of last follow-up.

For patients who exhibited skin reactions, there were no grade 5 events and 1 grade 4 event. There were 13 patients with severe cutaneous adverse reactions (i.e., having a range of skin reaction preferred terms, irrespective of grade), most of which were ≤Grade 2, with no grade 4 or 5 events observed. Of these 13 patients with severe cutaneous adverse reactions, 4 patients had grade 3 events as follows: stomatitis, skin exfoliation, dermatitis bullous, and dermatitis exfoliative generalized. In addition, one patient discontinued treatment due to grade 3 dermatitis bullous.

Similar rates of peripheral neuropathy were observed in patients with and without preexisting peripheral neuropathy (60% vs 55%, respectively).

A higher rate of hyperglycemia was observed in patients with preexisting hyperglycemia relative to patients without preexisting hyperglycemia (20% vs. 7%, respectively).

Four treatment-related deaths were previously reported and described in Section 6.1 (Example 1) (acute kidney injury, metabolic acidosis, multiple organ dysfunction syndrome, and pneumonitis). No additional treatment-related deaths were reported in the updated analysis for the three-month follow-up study.

Taken together, the three-month follow-up results above confirmed the results from the primary analysis in Section 6.1. First, the majority of platinum-naïve, cis-ineligible patients la/mUC patients who progressed on/after PD-1/L1 inhibitors achieved durable responses to enfortumab vedotin, with 22% achieving CR. Second. DOR, PFS, and OS continue to be encouraging, with no new safety signals.

In conclusion, based on the robust efficacy and manageable safety profile in cisplatin-ineligible patients with advanced urothelial cancer, who received prior PD-1/PD-L1, the benefit risk profile of enfortumab vedotin is positive.

What is claimed is:

1. A method of preventing or treating cancer in a human subject, comprising administering to the subject an effective amount of an antibody drug conjugate,
wherein the antibody drug conjugate comprises an antibody or antigen binding fragment thereof that binds to 191P4D12 conjugated to one or more units of monomethyl auristatin E (MMAE);
wherein the subject has urothelial or bladder cancer;
wherein the subject has received an immune checkpoint inhibitor (CPI) therapy;
wherein the subject is ineligible to receive cisplatin treatment (cisplatin ineligible).

2. The method of claim 1, wherein the cisplatin ineligible subject is a platinum-naïve subject.

3. The method of claim 1 or 2, wherein the platinum-naïve subject is a subject that received platinum in the adjuvant or neoadjuvant setting and did not progress within 12 months of completion of the platinum treatment.

4. The method of claim 1 or 2, wherein the platinum-naïve subject is a subject that has not received prior platinum-containing or other chemotherapy in the locally advanced or metastatic setting.

5. The method of any one of claims 1 to 4, wherein the cisplatin ineligible subject has one or more of the conditions selected from the group consisting of: ECOG performance status score of 2, impaired renal function, and no less than Grade 2 hearing loss.

6. The method of claim 5, wherein the impaired renal function is determined by creatinine clearance (CrCl) less than 60 mL/min.

7. The method of claim 5, wherein the impaired renal function is determined by CrCl less than 60 but no less than 30 mL/min.

8. The method of claim 5, wherein the impaired renal function is determined by CrCl less than 30 but no less than 15 mL/min.

9. The method of any one of claims 1 to 8, wherein the cisplatin ineligible subject had progression or recurrence of the cancer during or following most recent therapy.

10. The method of any one of claims 1 to 8, wherein the cisplatin ineligible subject had progression or recurrence of the cancer during or following the CPI therapy.

11. The method of any one of claims 1 to 10, wherein the subject has a primary site of tumor in the lower urinary tract.

12. The method of any one of claims 1 to 10, wherein the subject has a primary site of tumor in the upper urinary tract.

13. The method of any one of claims 1 to 12, wherein the subject has visceral metastases.

14. The method of any one of claims 1 to 13, wherein the subject has liver metastases.

15. The method of any one of claims 1 to 14, wherein the subject has at least 1 Bellmunt risk factor.

16. The method of any one of claims 1 to 15, wherein the subject has one or more of the conditions selected from the group consisting of:
(i) absolute neutrophil count no less than 1.0×10⁹/L,
(ii) platelet count no less than 100×10⁹/L,
(iii) hemoglobin no less than 9 g/dL,
(iv) serum bilirubin no more than either of 1.5 times of upper limit of normal (ULN) or 3 times ULN for patients with Gilbert's disease,
(v) CrCl no less than 30 mL/min, and
(vi) alanine aminotransferase and aspartate aminotransferase no more than 3 fold of ULN.

17. The method of claim 16, wherein the subject has all of conditions (i) to (vi) of claim 16.

18. The method of any one of claims 6 to 8, 16 and 17, wherein the CrCl is measured by 24 hour urine collection or estimated by the Cockcroft-Gault criteria.

19. The method of any one of claims 1 to 18, wherein the subject has no more than Grade 2 sensory or motor neuropathy.

20. The method of any one of claims 1 to 19, wherein the subject has no active central nervous system metastases.

21. The method of any one of claims 1 to 20, wherein the subject has no uncontrolled diabetes.

22. The method of claim 21, wherein the uncontrolled diabetes is determined by hemoglobin A1c (HbA1c) no less than 8% or HbA1c between 7 and 8% with associated diabetes symptoms that are not otherwise explained.

23. The method of claim 22, wherein the associated diabetes symptoms comprise or consist of polyuria, polydipsia, or both polyuria and polydipsia.

24. The method of any one of claims 1 to 23, wherein the subject has locally advanced or metastatic urothelial cancer.

25. The method of any one of claims 1 to 23, wherein the subject has locally advanced or metastatic bladder cancer.

26. The method of any one of claims 1 to 25, wherein the CPI therapy is a therapy of programmed death receptor-1 (PD-1) inhibitor.

27. The method of any one of claims 1 to 25, wherein the CPI therapy is a therapy of programmed death-ligand 1 (PD-L1) inhibitor.

28. The method of claim 26, wherein PD-1 inhibitor is nivolumab or pembrolizumab.

29. The method of claim 27, wherein PD-L1 inhibitor is selected from a group consisting of atezolizumab, avelumab, and durvalumab.

30. The method of any one of claims 1 to 29, wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

31. The method of any one of claims 1 to 30, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO: 16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

32. The method of any one of claims 1 to 30, wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

33. The method of any one of claims 1 to 32, wherein the antibody or antigen binding fragment thereof comprises a heavy chain variable region comprising the amino acid sequence of SEQ ID NO:22 and a light chain variable region comprising the amino acid sequence of SEQ ID NO:23.

34. The method of any one of claims 1 to 33, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

35. The method of any one of claims 1 to 33, wherein the antigen binding fragment is an Fab, F(ab′)2, Fv or scFv.

36. The method of any one of claims 1 to 34, wherein the antibody is a fully human antibody.

37. The method of any one of claims 1 to 34 and 36, wherein the antibody is an IgG1 and light chain is a kappa light chain

38. The method of any one of claims 1 to 37, wherein the antibody or antigen binding fragment thereof is recombinantly produced.

39. The method of any one of claims 1 to 38, wherein the antibody or antigen binding fragment is conjugated to each unit of MMAE via a linker.

40. The method of claim 39, wherein the linker is an enzyme-cleavable linker, and wherein the linker forms a bond with a sulfur atom of the antibody or antigen binding fragment thereof.

41. The method of claim 39 or 40, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

42. The method of claim 41, wherein the stretcher unit has the structure of Formula (1) below; the amino acid unit is valine-citrulline; and the spacer unit is a PAB group comprising the structure of Formula (2) below:

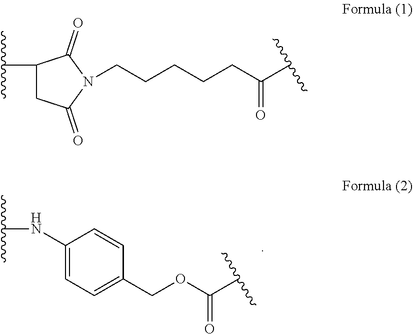 [SMILES: *C(=O)CCCCCN1C(=O)CC(*)C1=O ; *Nc1ccc(COC(*)=O)cc1]

43. The method of claim 41 or 42, wherein the stretcher unit forms a bond with a sulfur atom of the antibody or antigen binding fragment thereof; and wherein the spacer unit is linked to MMAE via a carbamate group.

44. The method of any one of claims 1 to 43, wherein the ADC comprises from 1 to 20 units of MMAE per antibody or antigen binding fragment thereof.

45. The method of any one of claims 1 to 44, wherein the ADC comprises from 1 to 10 units of MMAE per antibody or antigen binding fragment thereof.

46. The method of any one of claims 1 to 45, wherein the ADC comprises from 2 to 8 units of MMAE per antibody or antigen binding fragment thereof.

47. The method of any one of claims 1 to 46, wherein the ADC comprises from 3 to 5 units of MMAE per antibody or antigen binding fragment thereof.

48. The method of any one of claims 1 to 45, wherein the ADC has the following structure: 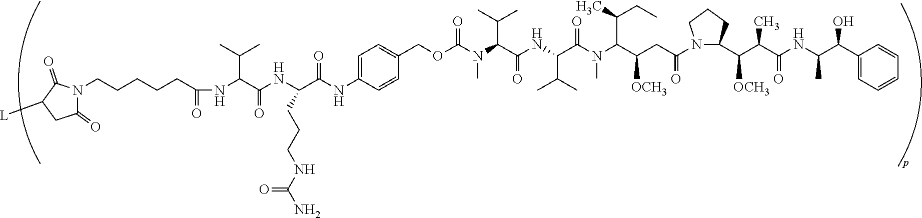
wherein L- represents the antibody or antigen binding fragment thereof and p is from 1 to 10.

49. The method of claim 48, wherein p is from 2 to 8.

50. The method of claim 48 or 49, wherein p is from 3 to 5.

51. The method of any one of claims 48 to 50, wherein p is from 3 to 4.

52. The method of any one of claims 48 to 51, wherein p is about 4.

53. The method of any one of claims 48 to 51, wherein the average p value of the effective amount of the antibody drug conjugates is about 3.8.

54. The method of any one of claims 1 to 53, wherein the ADC is administered at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

55. The method of any one of claims 1 to 54, wherein the ADC is administered at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

56. The method of any one of claims 1 to 55, wherein the ADC is administered at a dose of about 1 mg/kg of the subject's body weight.

57. The method of any one of claims 1 to 55, wherein the ADC is administered at a dose of about 1.25 mg/kg of the subject's body weight.

58. The method of any one of claims 1 to 57, wherein the ADC is administered by an intravenous (IV) injection or infusion.

59. The method of any one of claims 1 to 58, wherein the ADC is administered by an IV injection or infusion three times every four-week cycle.

60. The method of any one of claims 1 to 59, wherein the ADC is administered by an IV injection or infusion on Days 1, 8 and 15 of every four-week cycle.

61. The method of any one of claims 1 to 60, wherein the ADC is administered by an IV injection or infusion over about 30 minutes three times every four-week cycle.

62. The method of any one of claims 1 to 61, wherein the ADC is administered by an IV injection or infusion over about 30 minutes on Days 1, 8 and 15 of every four-week cycle.

63. The method of any one of claims 1 to 62, wherein the ADC is formulated in a pharmaceutical composition comprising L-histidine, polysorbate-20 (TWEEN-20), and trehalose dehydrate.

64. The method of any one of claims 1 to 63, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

65. The method of any one of claims 1 to 63, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

66. The method of any one of claims 1 to 65, wherein the ADC has the following structure: 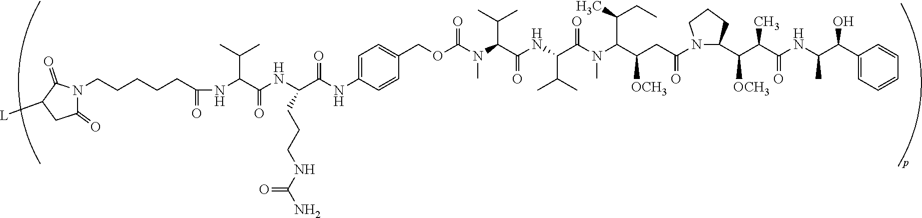
wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on Days 1, 8 and 15 of every four-week cycle.

67. The method of any one of claims 1 to 66, whereby the subject has a complete response following the treatment.

68. The method of any one of claims 1 to 66, wherein the subject has a partial response following the treatment.

69. The method of any one of claims 1 to 66, wherein the subject has a complete response or a partial response following the treatment.

70. The method of any one of claims 1 to 66, wherein the subject has a stable disease following the treatment.

71. The method of any one of claims 1 to 66, wherein the subject has a duration of response of at least or about 10 months following the treatment.

72. The method of any one of claims 1 to 66, wherein the subject has a duration of response ranging from 5 to 22 months following the treatment.

73. The method of any one of claims 1 to 66, wherein the subject has a progression free survival of at least or about 5 months following the treatment.

74. The method of any one of claims 1 to 66, wherein the subject has a progression free survival ranging from 5 to 9 months following the treatment.

75. The method of any one of claims 1 to 66, wherein the subject has an overall survival of at least or about 14 months following the treatment.

76. The method of any one of claims 1 to 66, wherein the subject has an overall survival ranging from 10 to 19 months following the treatment.

77. The method of any one of claims 1 to 66, wherein a population of the subjects is treated by the methods, and wherein percentage of the subjects having complete response in the treated population is at least or about 20%.

78. The method of any one of claims 1 to 66, wherein a population of the subjects is treated by the methods, and wherein percentage of the subjects having partial response in the treated population is at least or about 31%.

79. The method of any one of claims 1 to 66, wherein a population of the subjects is treated by the methods, and wherein objective response rate in the treated population is at least or about 51%.

80. The method of any one of claims 1 to 66, wherein a population of the subjects is treated by the methods, and wherein objective response rate in the treated population ranges from 40% to 63%.

81. The method of any one of claims 1 to 66, wherein a population of the subjects is treated by the methods, and wherein percentage of the subjects having stable disease in the treated population is at least or about 30%.

82. The method of any one of claims 1 to 66, wherein a population of the subjects is treated by the methods, and wherein median duration of response in the treated population is at least or about 10 months.

83. The method of any one of claims 1 to 66, wherein a population of the subjects is treated by the methods, and wherein duration of response in the treated population ranges from 5 to 22 months.

84. The method of any one of claims 1 to 66, wherein a population of the subjects is treated by the methods, and wherein median progression free survival in the treated population is at least or about 5 months.

85. The method of any one of claims 1 to 66, wherein a population of the subjects is treated by the methods, and wherein progression free survival in the treated population ranges from 5 to 9 months.

86. The method of any one of claims 1 to 66, wherein a population of the subjects is treated by the methods, and wherein median overall survival in the treated population is at least or about 14 months.

87. The method of any one of claims 1 to 66, wherein a population of the subjects is treated by the methods, and wherein overall survival in the treated population ranges from 10 to 19 months.

88. The method of any one of claims 1 to 67 and 69, wherein the complete response rate is at least or about 20% for a population of subjects treated with the method.

89. The method of any one of claims 1 to 66, 68, and 69, wherein the partial response rate is at least or about 31% for a population of subjects treated with the method.

90. The method of any one of claims 1 to 69, wherein objective response rate is at least or about 51% for a population of subjects treated with the method.

91. The method of any one of claims 1 to 69, wherein objective response rate is from 40% to 63% for a population of subjects treated with the method.

92. The method of any one of claims 1 to 66 and 70, wherein the stable disease rate is at least or about 30% for a population of subjects treated with the method.

93. The method of any one of claims 1 to 66, 71 and 72, wherein the median duration of response is at least or about 10 months for a population of subjects treated with the method.

94. The method of any one of claims 1 to 66, 71 and 72, wherein the duration of response is from 5 to 22 months for a population of subjects treated with the method.

95. The method of any one of claims 1 to 66, 73 and 74, wherein the median progression free survival is at least or about 5 months for a population of subjects treated with the method.

96. The method of any one of claims 1 to 66, 73 and 74, wherein the progression free survival is from 5 to 9 months for a population of subjects treated with the method.

97. The method of any one of claims 1 to 66, 75 and 76, wherein the median overall survival is at least or about 14 months for a population of subjects treated with the method.

98. The method of any one of claims 1 to 66, 75 and 76, wherein the overall survival is from 10 to 19 months for a population of subjects treated with the method.


SEQUENCE LISTING (23 sequences)

SEQ ID NO: 1 - DNA, 3464 bp, Homo sapiens
  CDS (264)...(1796)
  misc_feature (1)...(3464): 191P4D12
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  misc_feature (1)...(510): 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  CDS (32)...(1432)
  misc_feature (1)...(1432): Ha22-2(2,4)6.1 heavy chain
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  misc_feature (1)...(466): Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  CDS (25)...(735)
  misc_feature (1)...(735): Ha22-2(2,4)6.1 light chain
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  misc_feature (1)...(236): Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  misc_feature (1)...(466): Ha22-2(2,4)6.1 heavy chain

SEQ ID NO: 8 - protein, 236 aa, Homo sapiens; identical to SEQ ID NO: 6
  misc_feature (1)...(236): Ha22-2(2,4)6.1 light chain

SEQ ID NO: 9 - protein, 5 aa, Homo sapiens
  CDR1 of heavy chain
SYNMN

SEQ ID NO: 10 - protein, 17 aa, Homo sapiens
  CDR2 of heavy chain
YISSSSSTIYYADSVKG

SEQ ID NO: 11 - protein, 8 aa, Homo sapiens
  CDR3 of heavy chain
AYYYGMDV

SEQ ID NO: 12 - protein, 11 aa, Homo sapiens
  CDR1 of light chain
RASQGISGWLA

SEQ ID NO: 13 - protein, 7 aa, Homo sapiens
  CDR2 of light chain
AASTLQS

SEQ ID NO: 14 - protein, 9 aa, Homo sapiens
  CDR3 of light chain
QQANSFPPT

SEQ ID NO: 15 - protein, 4 aa, Artificial Sequence
  linker
GFLG

SEQ ID NO: 16 - protein, 8 aa, Artificial Sequence
  VH CDR1 according to IMGT
GFTFSSYN

SEQ ID NO: 17 - protein, 8 aa, Artificial Sequence
  VH CDR2 according to IMGT
ISSSSSTI

SEQ ID NO: 18 - protein, 10 aa, Artificial Sequence
  VH CDR3 according to IMGT
ARAYYYGMDV

SEQ ID NO: 19 - protein, 6 aa, Artificial Sequence
  VL CDR1 according to IMGT
QGISGW

SEQ ID NO: 20 - protein, 3 aa, Artificial Sequence
  VL CDR2 according to IMGT
AAS

SEQ ID NO: 21 - protein, 9 aa, Artificial Sequence
  VL CDR3 according to IMGT
QQANSFPPT

SEQ ID NO: 22 - protein, 117 aa, Artificial Sequence
  heavy chain variable region (VH), the 20th amino acid (glutamic acid) to the 136th amino acid (serine) of SEQ ID NO7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, Artificial Sequence
  light chain variable region (VL), the 23rd amino acid (aspartic acid) to the 130th amino acid (arginine) of SEQ ID NO8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR